(19) United States
(12) Patent Application Publication
(10) Pub. No.: US 2025/0145690 A1
(43) Pub. Date: May 8, 2025

(54) HUMAN MONOCLONAL ANTIBODIES THAT BROADLY TARGET CORONAVIRUSES

(71) Applicant: The U.S.A., as represented by the Secretary, Department of Health and Human Services, Bethesda, MD (US)

(72) Inventors: Joshua Hoong Yu Tan, Rockville, MD (US); Cherrelle Dacon, Rockville, MD (US); Courtney Tucker, Reston, VA (US)

(73) Assignee: The U.S.A., as represented by the Secretary, Department of Health and Human Services, Bethesda, MD (US)

(21) Appl. No.: 18/835,912

(22) PCT Filed: Feb. 9, 2023
(86) PCT No.: PCT/US2023/062324

§ 371 Date: Aug. 5, 2024

Related U.S. Application Data
(60) Provisional application No. 63/308,898, filed on Feb. 10, 2022.

(51) Int. Cl. C07K 16/10; A61K 39/00; A61P 37/04

(52) U.S. Cl. CPC C07K 16/1003; A61P 37/04; A61K 2039/505; C07K 2317/24; C07K 2317/33; C07K 2317/76

(57) ABSTRACT

Disclosed are monoclonal antibodies and antigen binding fragments that specifically bind a coronavirus spike protein, such as SARS-CoV-2. Also disclosed is the use of these antibodies for inhibiting a coronavirus infection. In addition, disclosed are methods for detecting a coronavirus in a biological sample, using the disclosed antibodies.

### CROSS REFERENCE TO RELATED APPLICATIONS

This claims the benefit of U.S. Provisional Application No. 63/308,898, filed Feb. 10, 2022, which is incorporated by reference herein in its entirety.

### FIELD OF THE DISCLOSURE

This relates to monoclonal antibodies and antigen binding fragments that specifically bind a coronavirus spike protein, and their use for inhibiting or detecting a coronavirus infection in a subject.

### REFERENCE TO AN ELECTRONIC SEQUENCE LISTING

The contents of the electronic sequence listing (4239-107907-02 ST26 SL.xml; Size: 432,282 bytes; and Date of Creation: Feb. 4, 2023) is herein incorporated by reference in its entirety.

### BACKGROUND

The novel coronavirus SARS-CoV-2 emerged in in 2019 and has infected 400 million individuals and caused more than 5 million deaths worldwide. While effective vaccines and antibody therapeutics have been developed, their efficacy is threatened by the emergence of variants of concern, including the Omicron variant, which is more resistant than previous variants to many of these tools. Furthermore, the possibility of further coronavirus spillover from animal reservoirs remains and must be prepared for. A need remains for antibodies that bind multiple coronaviruses.

### SUMMARY OF THE DISCLOSURE

A panel of human monoclonal antibodies (mAbs) that target multiple coronaviruses were isolated. These mAbs can be used to inhibit a coronavirus infection, as prophylactics to prevent coronavirus infection, and as tools for the development of next-generation vaccines that protect more broadly against coronaviruses.

Isolated monoclonal antibody or antigen binding fragments are disclosed that specifically bind to a coronavirus spike protein and neutralize SARS-CoV-2 and at least one additional betacoronavirus or alphacoronavirus.

Isolated monoclonal antibodies or antigen binding fragments thereof are disclosed that specifically binds to a coronavirus spike protein. These monoclonal antibodies or antigen binding fragments can neutralize at least one coronavirus.

In some embodiments, the antigen or antigen binding fragment includes a heavy chain variable (V_{H}) region and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as any one of:
a) SEQ ID NOs: 17 and 21, respectively (COV44-79);
b) SEQ ID NOs: 9 and 13, respectively (COV44-62);
c) SEQ ID NOs: 1 and 5, respectively (COV89-22);
d) SEQ ID NOs: 25 and 29, respectively (COV30-14);
e) SEQ ID NOs: 33 and 37, respectively (COV72-37);
f) SEQ ID NOs: 49 and 53, respectively (COV91-27);
g) SEQ ID NOs: 41 and 45, respectively (COV93-03);
h) SEQ ID NOs: 57 and 61, respectively (COV49-51);
i) SEQ ID NOs: 65 and 69, respectively (COV44-74);
j) SEQ ID NOs: 73 and 77, respectively (COV44-56);
k) SEQ ID NOs: 81 and 85, respectively (COV44-26);
l) SEQ ID NOs: 89 and 93, respectively (COV44-54);
m) SEQ ID NOs: 97 and 101, respectively (COV23-01);
n) SEQ ID NOs: 105 and 109, respectively (COV49-03);
o) SEQ ID NOs: 113 and 117, respectively (COV49-04);
p) SEQ ID NOs: 121 and 125, respectively (COV49-05);
q) SEQ ID NOs: 129 and 133, respectively (COV49-06);
r) SEQ ID NOs: 137 and 141, respectively (COV49-07);
s) SEQ ID NOs: 145 and 149, respectively (COV49-18);
t) SEQ ID NOs: 153 and 157, respectively (COV49-23);
u) SEQ ID NOs: 161 and 165, respectively (COV49-28);
v) SEQ ID NOs: 169 and 173, respectively (COV49-30);
w) SEQ ID NOs: 177 and 181, respectively (COV49-33);
x) SEQ ID NOs: 185 and 189, respectively (COV49-42);
y) SEQ ID NOs: 193 and 197, respectively (COV49-47);
z) SEQ ID NOs: 201 and 205, respectively (COV49-54);
aa) SEQ ID NOs: 209 and 213, respectively (COV57-01);
bb) SEQ ID NOs: 217 and 221, respectively (COV57-03);
cc) SEQ ID NOs: 225 and 229, respectively (COV57-04);
dd) SEQ ID NOs: 233 and 237, respectively (COV57-05);
ee) SEQ ID NOs: 241 and 245, respectively (COV57-13);
ff) SEQ ID NOs: 249 and 253, respectively (COV57-19);
gg) SEQ ID NOs: 257 and 261, respectively (COV57-34);
hh) SEQ ID NOs: 265 and 269, respectively (COV57-38);
ii) SEQ ID NOs: 273 and 277, respectively (COV57-45);
jj) SEQ ID NOs: 281 and 285, respectively (COV77-02);
kk) SEQ ID NOs: 289 and 293, respectively (COV77-04);
ll) SEQ ID NOs: 297 and 301, respectively (COV77-05);
mm) SEQ ID NOs: 305 and 309, respectively (COV77-09);
nn) SEQ ID NOs: 313 and 317, respectively (COV77-14);
oo) SEQ ID NOs: 321 and 325, respectively (COV77-35);
pp) SEQ ID NOs: 329 and 333, respectively (COV77-39);
qq) SEQ ID NOs: 337 and 341, respectively (COV77-42);
rr) SEQ ID NOs: 345 and 349, respectively (COV77-43);
ss) SEQ ID NOs: 353 and 357, respectively (COV77-46);
tt) SEQ ID NOs: 361 and 365, respectively (COV77-76);
uu) SEQ ID NOs: 369 and 373, respectively (COV93-04);
vv) SEQ ID NOs: 377 and 381, respectively (COV93-08);
ww) SEQ ID NOs: 385 and 389, respectively (COV93-17);
xx) SEQ ID NOs: 393 and 397, respectively (COV93-18);
yy) SEQ ID NOs: 401 and 405, respectively (COV93-23);
zz) SEQ ID NOs: 409 and 413, respectively (COV78-36);
aaa) SEQ ID NOs: 417 and 421, respectively (COV93-38);
bbb) SEQ ID NOs: 425 and 429, respectively (COV93-60);
ccc) SEQ ID NOs: 433 and 437, respectively (COV93-61);
ddd) SEQ ID NOs: 441 and 445, respectively (COV89-03);
eee) SEQ ID NOs: 449 and 453, respectively (COV89-28);
fff) SEQ ID NOs: 457 and 461, respectively (COV30-35);
ggg) SEQ ID NOs: 465 and 469, respectively (COV30-80);
hhh) SEQ ID NOs: 473 and 477, respectively (COV44-25); or
iii) SEQ ID NOs: 481 and 485, respectively (COV44-37).

In some embodiments, disclosed are nucleic acid molecules encoding these antibodies and antigen binding fragments, vectors including these nucleic acid molecules, and host cells including these vectors.

Pharmaceutical compositions including the antibodies, antigen binding fragments, nucleic acid molecules, and vectors are also disclosed. In more embodiments, disclosed is the use of these pharmaceutical compositions to inhibit a coronavirus infection in a subject.

In yet other embodiments, disclosed is the use of the disclosed antibodies and antigen binding fragments for the detection of a coronavirus in a biological sample.

The foregoing and other features and advantages of the invention will become more apparent from the following detailed description of several embodiments which proceeds with reference to the accompanying figures.

### BRIEF DESCRIPTION OF THE FIGURES

FIG. 1: Broadly reactive antibodies target multiple human coronaviruses. Heat map representing the binding of the broadly reactive mAbs to spike proteins of diverse coronaviruses. Binding is shown for SARS-CoV-2 Wuhan Hu-1, SARS-CoV-1, MERS-CoV, HCoV-HKU1, HCoV-OC43 (betacoronavirus genus); HCoV-NL63 and HCoV-229E (alphacoronavirus genus) and H1 hemagglutinin (control). The malaria mAb L9 was included as a negative control for mAb binding experiments (Wang, L. T., et al. 2020). Area under the curve (AUC) values for each antigen are shown after subtraction with values for the negative control antigen CD4.

FIG. 2: The SARS-CoV-2 spike protein S2 subunit is a common target for broadly reactive antibodies. The heat map shows values for binding to selected domains within the SARS-CoV-2 spike protein: the receptor binding domain (RBD), N-terminal domain (NTD) and the S2 subunit (representative of n=1 experiment). Area under the curve (AUC) values are shown after subtraction with values for the negative control antigen CD4.

FIG. 3: Broadly reactive mAbs target distinct epitopes within the SARS-CoV-2 S2 subunit. Surface plasmon resonance (SPR)-based analyses of binding to recombinant SARS-CoV-2 S2 subunit identified three distinct epitope bins for the broadly reactive mAbs (numbered in inset). Dark grey boxes indicate competing antibody pairs, light grey boxes indicate non-competing antibody pairs and hashed filling indicates self-competition.

FIGS. 4A-4B. Antibody binding to the S2 fusion peptide, stem-helix region and K814⁺ site. SPR-based peptide scanning analyses identified the S2 fusion peptide (FP), stem helix (SH) and a site N-terminal to the S2 cleavage site and fusion peptide region (K814+) as the three distinct epitopes targeted by the broadly reactive mAbs. The heat map represents binding to an array of 15-mer peptides with 12 amino acid overlay covering the entire S2 subunit for (A) fusion peptide mAbs and (B) stem helix mAbs. Each column within the heat map represents a single peptide. The white triangle shows the S1/S2 cleavage site and the black triangle indicates the S2′ cleavage site. FP, fusion peptide; HR1, heptad repeat 1; C helix, central helix; CD, connector domain; SH, stem helix; HR2, heptad repeat 2. In FIG. 4A, SEQ ID NO: 491 is the amino acid sequence of Peptide 42; SEQ ID NO: 492 is the amino acid sequence of Peptide 43; and SEQ ID NO: 493 is the amino acid sequence of Peptide 44. In FIG. 4B, SEQ ID NO: 494 is the amino acid sequence of Peptide 153; SEQ ID NO: 495 is the amino acid sequence of Peptide 154; and SEQ ID NO: 496 is the amino acid sequence of Peptide 155.

FIGS. 5A-5F. Broadly reactive antibodies target highly conserved regions within coronavirus spike proteins. (A) Sequence conservation of the fusion peptide determined by alignment of spike protein sequences of 34 viral isolates representing a diverse group of coronaviruses across four genera. (B) Sequence conservation of the stem helix region determined alignment of spike protein sequences of 28 viral isolates representing each of the five betacoronavirus subgenera. Heat map coloring denotes percent identity of amino acid residues. (C-F) Peptide sequences used in FIGS. 5A and 5B are provided as SEQ ID NOs: 499-562, see also FIGS. 5C to 5F.

FIGS. 6A-6B. Broadly reactive antibodies neutralize multiple human coronaviruses and SARS-CoV-2 variants of concern. Neutralization of SARS-CoV-2 wild-type (WT) and variants of concern (pseudovirus) by (A) fusion peptide mAbs COV44-62 and COV44-79, and (B) stem-helix mAbs COV30-14, COV72-37 and COV89-22. The dotted line indicates 50% neutralization. Error bars show mean±SD.

FIGS. 7A-7B. Broadly reactive antibodies inhibit SARS-CoV-2 spike-mediated fusion with target cells. In vitro fusion inhibition by (A) fusion peptide mAbs (COV44-62, COV44-79, COV91-27, COV77-04, COV77-39 and COV78-36) and (B) stem helix-specific mAbs (COV30-14, COV72-37 and COV89-22) and K814+ specific mAbs (COV77-43 and COV93-18) in a quantitative assay. The malaria mAb L9 and CoV-2 RBD-specific mAb CV503 were used as a negative controls.

FIGS. 8A-8B. Broadly reactive antibodies limit SARS-CoV-2 mediated pathology in hamsters. Body weight changes in SARS-CoV-2 exposed Syrian hamsters after prophylaxis with individual (A) fusion peptide and (B) stem helix mAbs. Statistical analyses were carried out using a mixed-effects repeated measures model with Dunnett's post-test multiple comparison (n=12 animals from Day 0-3 and n=6 animals from Day 4-7) and error bars represent mean±SD. Bars show median±interquartile range. *P<0.05, **P<0.01, ***P<0.001, ****P<0.0001 and ns, not significant.

FIG. 9. Combinations of fusion peptide and stem helix antibodies limit SARS-CoV-2 mediated pathology in hamsters. Body weight changes in Syrian hamsters exposed to SARS-CoV-2 (BA.2) after prophylaxis with cocktails of fusion peptide and stem helix mAbs. Bars show median±interquartile range.

FIG. 10. Table 1. Cross-reactive antibodies neutralize multiple human coronaviruses. Neutralization curves of cross-reactive antibodies with SARS-CoV-2 Wuhan-Hu-1, SARS-CoV-2 omicron variant, SARS-CoV-1, MERS-CoV and NL63 envelope pseudotyped virus. Curves are from representative cross-reactive antibodies. Error bars represent mean with SEM and the dotted line represents 50% neutralization.

### SEQUENCES

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The complementarity determining regions (CDRs) are shown in bold in each variable heavy chain domain (V_{H}) and variable light chain domain (VL) shown below.

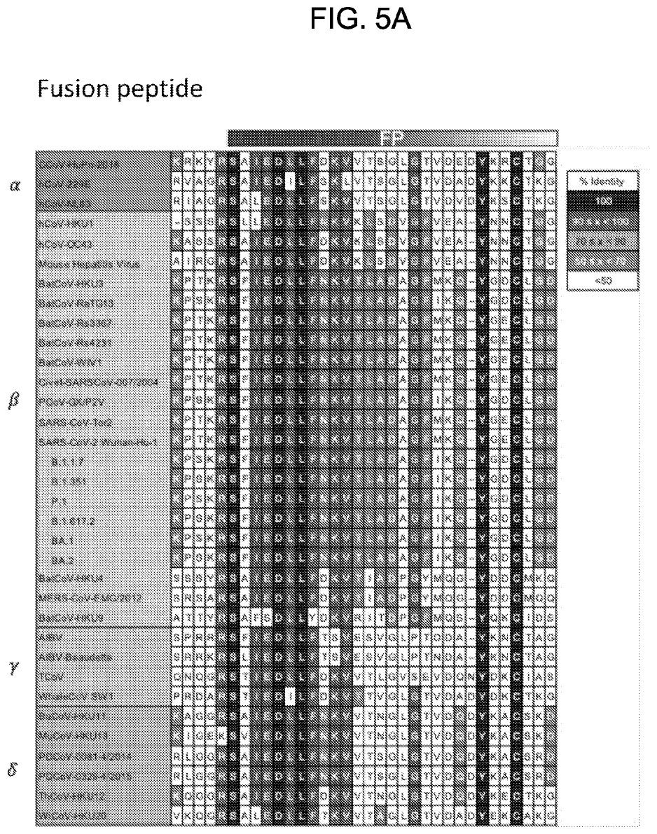

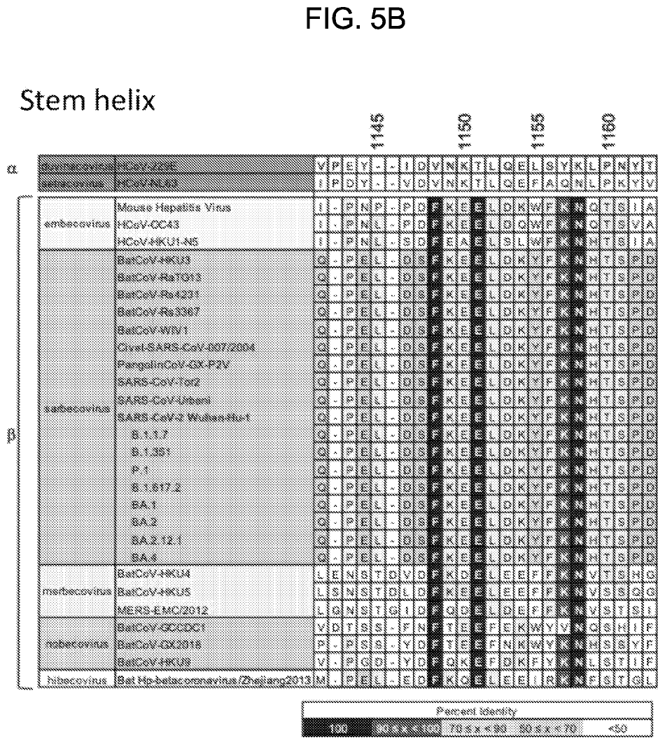

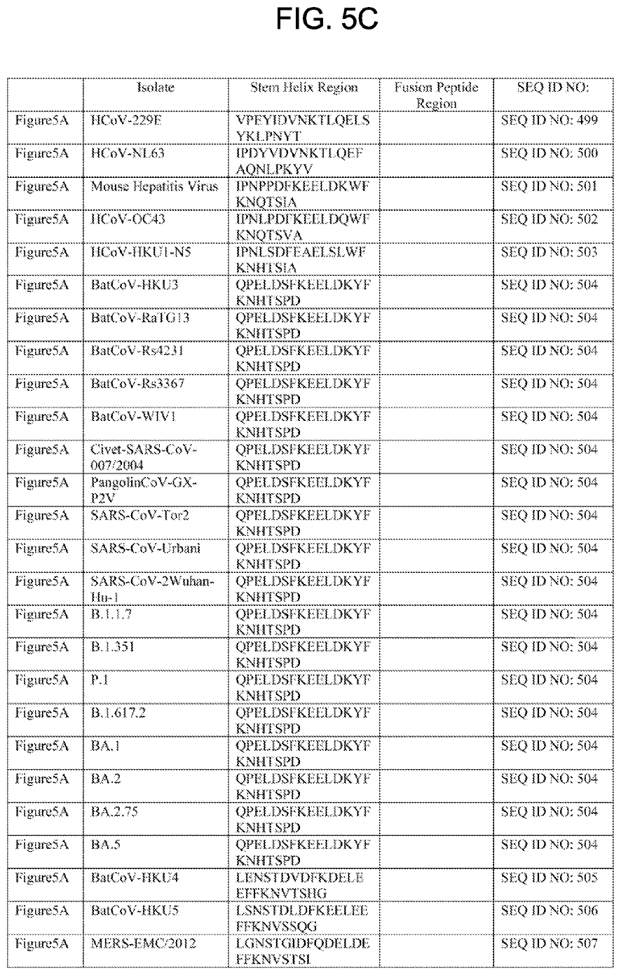

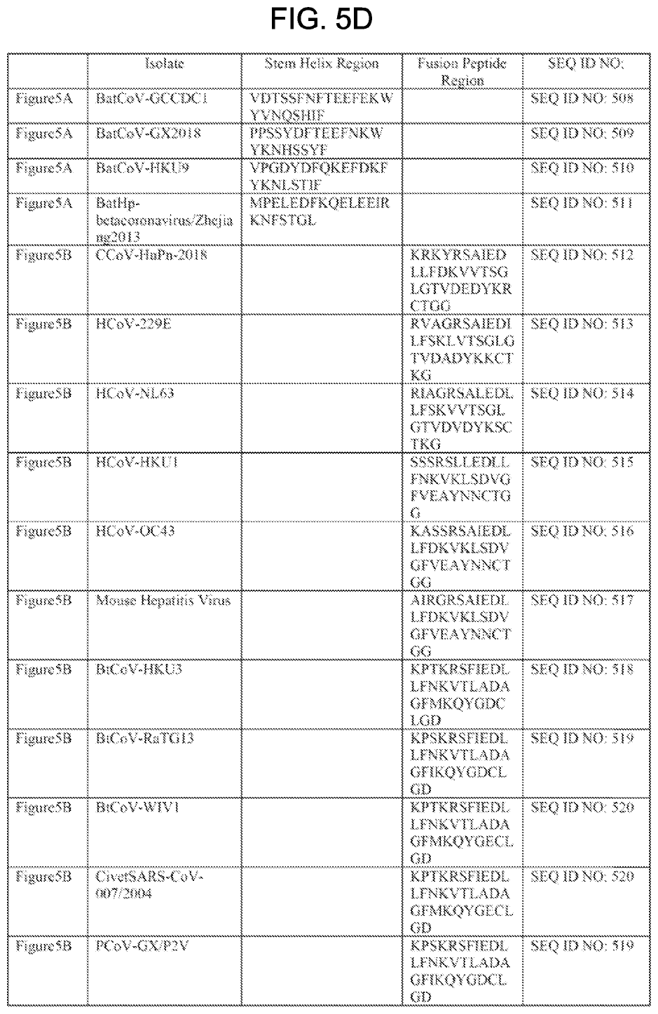

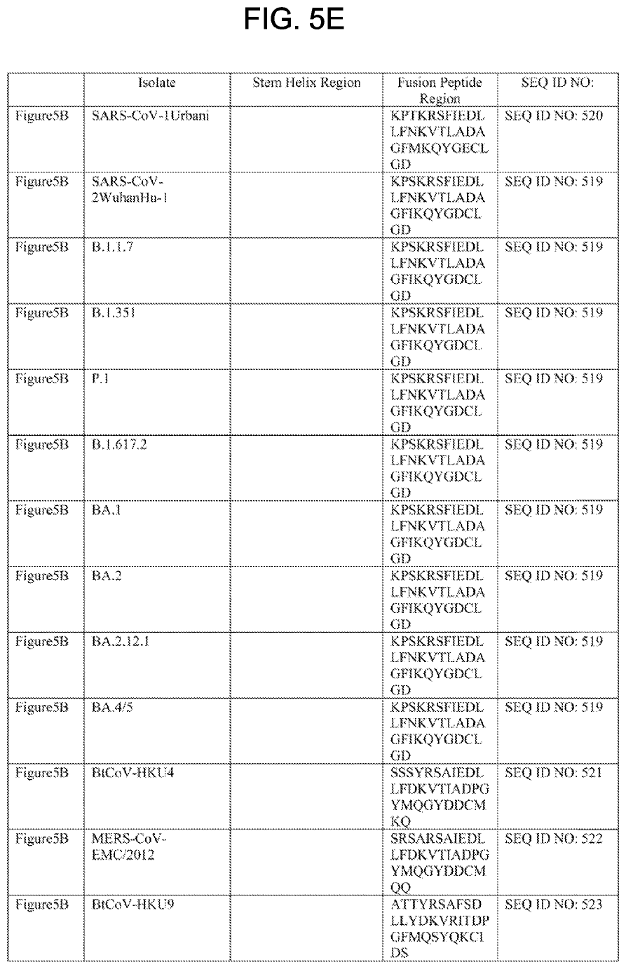

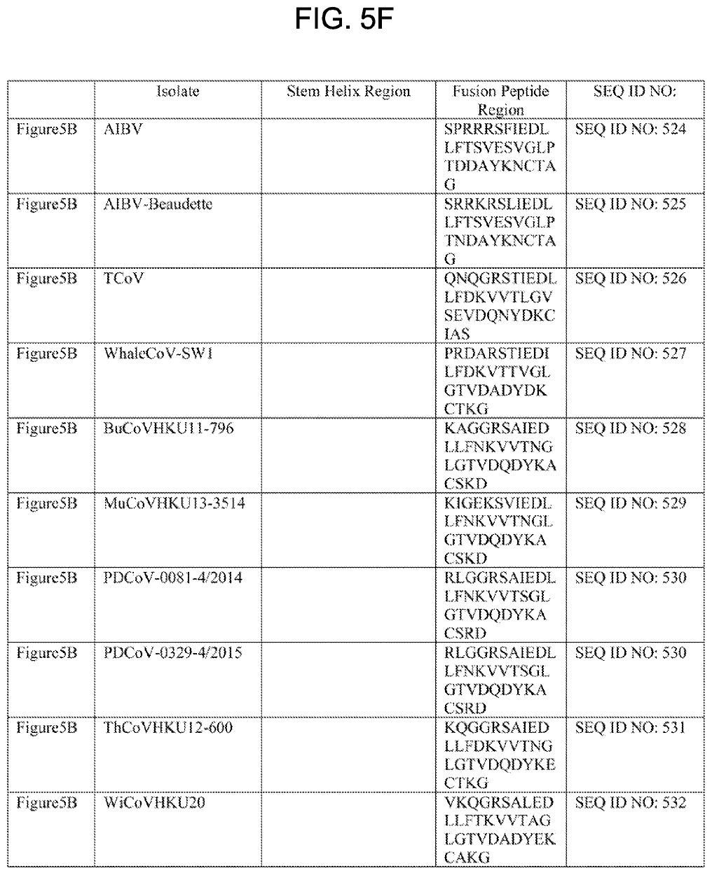

a) SEQ ID NO: 1 is the amino acid sequence of the COV89-22 V_{H}, SEQ ID NOs: 2, 3, and 4 are the
amino acid sequences of the HCDR1, HCDR2, and HCDR3, respectively.
QEQLVQSGAEVKKPGASVKVSCKSSGFTFSYFYLHWVRQAPGQGLEWMGIINPRGDGTRYAQKFQGRVTMTRDASTGTLY
MELRSLRSEDTAVYYCARGADHGAFDIWGQGTMVTVSS
SEQ ID NO: 5 is the amino acid sequence of the COV89-22V_{L}, SEQ ID NOs: 6, 7, and 8 are the
amino acid sequences of the LCDR1, LCDR2, and LCDR3, respectively.
EIVLTQSPGTLSLSPGERATLSCRASQSVRRNYFAWYQQKRGQAPRLLIYDASTRATGIPDRFSGSGSGTDFTLTISRLE
PEDFAVYYCQQYDSSPPMYIFGQGTKLEIK
b) SEQ ID NO: 9 is the amino acid sequence of the COV44-62 V_{H}, SEQ ID NOs: 10, 11 and 12 are
the amino acid sequences of the HCDR1, HCDR2, and HCDR3, respectively.
QMQLMQSGAEVKKPGASVTVSCKASGDTFSDYRIHWVRQAPGQGLEWMGRMNPKSGDTNFAQKFQGRVTMTRDMSINTAY
MTLSGLTFDDTALYYCASLLIVGGFDPLDDFEVWGQGTMVTISS
SEQ ID NO: 13 is the amino acid sequence of the COV44-62 V_{L}, SEQ ID NOs: 14, 15, and 16 are
the amino acid sequences of the LCDR1, LCDR2, and LCDR3, respectively.
QSALTQPPSASGSPGQSVTISCSGTSSDVGGYNFVSWYQHHPGKAPKILIYEVTKRPSGVPDRFSGSKSGNTASLTVSGL
QAEDEADYYCSSYGGTNNLLFGGGTKLTVL
c) SEQ ID NO: 17 is the amino acid sequence of the COV44-79 V_{H}, SEQ ID NOs: 18, 19, and 20
are the amino acid sequences of the HCDR1, HCDR2, and HCDR3, respectively.
QVQLVESGGGVVQPGRSLRLSCAASGLTFSGYAMHWVRQAPGKGLEWVAVISRDARNKYYADSVKGRFTISRDNSKKTVYLEMNSL
RVEDTAVYYCAILIIPGITEPGSPDALDIWGQGTMVSVSS
SEQ ID NO: 21 is the amino acid sequence of the COV44-79 V_{L}, SEQ ID NOs: 22, 23, and 24 are
the amino acid sequences of the LCDR1, LCDR2, and LCDR3, respectively.
DIQMTQSPSSMSASVGDRVTITCRASQDISKWLAWYQQRPGKAPKLLIYAASSLQSGVPSRFSGSGSGTDFTLTISSLQP
EDFATYYCQQASSFPWSITFGQGTRLEIR
d) SEQ ID NO: 25 is the amino acid sequence of the COV30-14 V_{H}, SEQ ID NOs: 26, 27, and 28
are the amino acid sequences of the HCDR1, HCDR2, and HCDR3, respectively.
QVQLVQSGAEVKKPGASVKVSCQTSGYTFTSYYMHWVRQAPGQGLEWMGLITPSGDDTYYAQRFQGRVTMTRDTSTSPTY
MELSSLTSEDTAVYYCAKMSRAGGFDVWGQGTLVTVSS
SEQ ID NO: 29 is the amino acid sequence of the COV30-14 V_{L}, SEQ ID NOs: 30, 31, and 32 are
the amino acid sequences of the LCDR1, LCDR2, and LCDR3, respectively.
EVVLTQSPGTLSLSPGERATLSCRASQSITGRYLAWYQQKPGQAPRLLMYGESSRVTGIPDRFSGGGSGTDFTLTISRLE
PEDFAVYYCQHFASSPPTYTFGQGTKLEIR
e) SEQ ID NO: 33 is the amino acid sequence of the COV72-37 V_{H}, SEQ ID NOs: 34, 35, and 36
are the amino acid sequences of the HCDR1, HCDR2, and HCDR3, respectively.
QVQLVQSGAEVKKPAASVKVSCKASGDTFSSHYMHWVRQAPGQGPEWMGIINPSGSGTAYGQKFQGRLTMTRDTSTSTVY
MELSSLTSDDTAVYYCGGGSGGLFAYWGQGTLVTVSS
SEQ ID NO: 37 is the amino acid sequence of the COV72-37 V_{L}, SEQ ID NOs: 38, 39, and 40 are
the amino acid sequences of the LCDR1, LCDR2, and LCDR3, respectively.
EIVLTQSPGTLSLSPGERATLSCRASQIVRSNYLAWYQQKPGQAPRLLIYGASSRATGTPDRFSGGGSGTDFTLTINRLE
PEDFAVYYCLQYDSSPPTYIFGQGTKLEIK
f) SEQ ID NO: 41 is the amino acid sequence of the COV93-03 V_{H}, SEQ ID NOs: 42, 43, and 44
are the amino acid sequences of the HCDR1, HCDR2, and HCDR3, respectively.
QLQLVQSGAEVKKPGASVKLSCKASGNTFTSHYIHWVRQAPGQGPEWMGIINPSGSGTRYAQKFQGRVTMTRDTSTSTVY
MELVSLRFEDTAVYYCAGDYYLDYWGQGTLVTVSS
SEQ ID NO: 45 is the amino acid sequence of the COV93-03 V_{L}, SEQ ID NOs: 46, 47, and 48 are
the amino acid sequences of the LCDR1, LCDR2, and LCDR3, respectively.
EFVLTQSPGTLSLSPGERATLSCRASQSVTSNYLAWYQQKPGQAPRLLIFGASSRATGIPDRFSGSGSGTDFTLTISRLE
PEDFAVYYCQQYGSSPPMYTFGPGTKVEIK
g) SEQ ID NO: 49 is the amino acid sequence of the COV91-27 V_{H}, SEQ ID NOs: 50, 51, and 52
are the amino acid sequences of the HCDR1, HCDR2, and HCDR3, respectively.
QVQLVESGGGVVHPGGSLRLSCAASGFTFSDFAMNWVRQAPGEGLQWVAIISYDGRNKHFAASVRGRFTISRDNSKNTMF
LQMSSLRAEDTAIYYCATLGGWFEEASPTYWGRGTLVTVSS
SEQ ID NO: 53 is the amino acid sequence of the COV91-27 V_{L}, SEQ ID NOs: 54, 55, and 56 are
the amino acid sequences of the LCDR1, LCDR2, and LCDR3, respectively.
EVVLTQSPAIVSLSPGERATLSCRASQSVGSRLAWHQQKPGLAPRVLIYDASNRATGIPARFSGSGSGTDFTLTISSIEP
EDSAVYYCQQRGDWPLITFGQGTRLEIK
h) SEQ ID NO: 57 is the amino acid sequence of the COV49-51 V_{H}, SEQ ID NOs: 58, 59, and 60
are the amino acid sequences of the HCDR1, HCDR2, and HCDR3, respectively.
QVQLVQSGAEAKKPGASVKVSCKASGYSFTSYHMHWVRQAPGQGLEWMGIINPNGGTTTYAPKFQGRVTMTSDTSTSTVYMELTSL
RSEDTAVYYCSTVVGALWGQGTLVIVSS
SEQ ID NO: 61 is the amino acid sequence of the COV49-51 V_{L}, SEQ ID NOs: 62, 63, and 64 are
the amino acid sequences of the LCDR1, LCDR2, and LCDR3, respectively.
IVLTQSPGTLSLSPGERATLSCRASQSVRSNNYLAWYQQKPGQAPRLLIYGASTRATGIPDRFSGSGSGTDFTLTISRLE
PEDFAVYYCQQYGSSPLTFGGGTRVEIK
i) SEQ ID NO: 65 is the amino acid sequence of the COV44-74 V_{H}, SEQ ID NOs: 66, 67, and 68
are the amino acid sequences of the HCDR1, HCDR2, and HCDR3, respectively.
QVQLVQSGAEVKKPGASVKVSCKASGYTFSSYYMNWVRQAPGQGLEWVGLISPSAGYTAYAQMFQGRVTMTRDTSTSTVY
MELSSLRSEDTAVYYCARVAVAGPNDYWGQGTLVTVSS
SEQ ID NO: 69 is the amino acid sequence of the COV44-74 V_{L}, SEQ ID NOs: 70, 71, and 72 are
the amino acid sequences of the LCDR1, LCDR2, and LCDR3, respectively.
EIVLTQSPGTLSLSPGERATLSCRASQSISSSYLAWYQQKPGQAPRLVILAASTRATGIPDRFSGSGSGTDFTLTISRLE
PEDFALYYCQHYRSSPPLWTFGQGTKVEIR
j) SEQ ID NO: 73 is the amino acid sequence of the COV44-56 V_{H}, SEQ ID NOs: 74, 75, and 76
are the amino acid sequences of the HCDR1, HCDR2, and HCDR3, respectively.
QVLLVQSGAEVKKPGASVMVSCKASGYILNNYYMHWVRQAPGHGLEWMGIINPSGDGTRYAQKFQGRVTMTRDSSTTSIH
MELRRVTSEDTAVYYCARGGSQSAFDMWGQGTVVLVSS
SEQ ID NO: 77 is the amino acid sequence of the COV44-56 V_{L}, SEQ ID NOs: 78, 79, and 80 are
the amino acid sequences of the LCDR1, LCDR2, and LCDR3, respectively.
DIVMTQSPRSLPVTPGESASISCRSSQSLLQKNGYNYLDWYVQKPGQSPQLLIYLGSYRASGVPDRISGSGSGTDETLKI
NRVEAEDVGVYYCLQAQQAPPLYTFGQGTKLQIE
k) SEQ ID NO: 81 is the amino acid sequence of the COV44-26 V_{H}, SEQ ID NOs: 82, 83, and 84
are the amino acid sequences of the HCDR1, HCDR2, and HCDR3, respectively.
QVQLVQSGAEVKKPGASVKVSCKASGYTFSNFYMNWVRQAPGQGLEWVGLISPSAGYTAYAQMFQGRVTMTRDTSTSTVY
MELSSLRSEDTAVYYCARVAVAGPNDYWGQGTLVTVSS
SEQ ID NO: 85 is the amino acid sequence of the COV44-26 V_{L}, SEQ ID NOs: 86, 87, and 88 are
the amino acid sequences of the LCDR1, LCDR2, and LCDR3, respectively.
EIVLTQSPGTLSLSPGERATLSCRASQSISSSYLAWYQQKPGQAPRLVIFAASTRATGIPDRFSGSGSGTDFTLTITRLE
PEDFALYYCQHYRSSPPLWTFGQGTKVEIR
l) SEQ ID NO: 89 is the amino acid sequence of the COV44-54 V_{H}, SEQ ID NOs: 90, 91, and 92
are the amino acid sequences of the HCDR1, HCDR2, and HCDR3, respectively.
QVQLVQSGAEVKKPGASVRVSCKTSGYTFTNYYMTWVRQAPGQGLQWVGLISPSAGYTAYAQMFQGRVTMTSDTSTSTVY
MQLSSLRSEDTAIYYCTRVAVAGPNDYWGQGTLVTVSS
SEQ ID NO: 93 is the amino acid sequence of the COV44-54 V_{L}, SEQ ID NOs: 94, 95, and 96 are
the amino acid sequences of the LCDR1, LCDR2, and LCDR3, respectively.
EIVLTQSPGTLSLSPGERATLSCRASQSISGSYLAWYQQKPGQAPRLVIYAASSRATGIPDRFSGSGSGTDFILTISRLE
AEDFAVYFCQHYRSSPPLWTFGQGTKVEIR
m) SEQ ID NO: 97 is the amino acid sequence of the COV23-01 V_{H}, SEQ ID NOs: 98, 99, and 100
are the amino acid sequences of the HCDR1, HCDR2, and HCDR3, respectively.
QVTLKESGPVLVKPTETLTLTCTVSGFSLSNTKMGVSWIRQPPGKALEWLAHIFSNDRKSYTTSLKSRLTISEDTSKSQI
VLTMTNMDPVDTATYYCARIGAGGTYNFYYYMDVWGKGTTVTVSS
SEQ ID NO: 101 is the amino acid sequence of the COV23-01 V_{L}, SEQ ID NOs: 102, 103, and 104
are the amino acid sequences of the LCDR1, LCDR2, and LCDR3, respectively.
EIVLTQSPATLSLSPGERATLSCRASQSVSSYLAWYQQKPGQAPRLLIYDASNRATGIPARFSGSGSGTDFTLTISSLEP
EDFAVYYCQQRSLYYTFGQGTKLEIK
n) SEQ ID NO: 105 is the amino acid sequence of the COV49-03 V_{H}, SEQ ID NOs: 106, 107, and
108 are the amino acid sequences of the HCDR1, HCDR2, and HCDR3, respectively.
DVQLLESGGGLVQPGGSRRLSCAASGFTFSNYAMSWVRQAPGKGLEWVSAISGGGATTYYADSVQGRFTMSRDNSNNTLY
LQMSSLRAEDTAVYYCTKGAFYDILSSIIVEGDYWGQGTLVTVSS
SEQ ID NO: 109 is the amino acid sequence of the COV49-03 V_{L}, SEQ ID NOs: 110, 111, and 112
are the amino acid sequences of the LCDR1, LCDR2, and LCDR3, respectively.
DVVLTQSPLSLPITLGQPASISCRSSQSLVFSDGNTYLNWFQQRPGQSPRRLIYKVSNRDSGVPDRFSGSGSGTDFTLKI
SRVEAEDVGVYYCMQGTHWLSITFGQGTRLEIK
o) SEQ ID NO: 113 is the amino acid sequence of the COV49-04 V_{H}, SEQ ID NOs: 114, 115, and
116 are the amino acid sequences of the HCDR1, HCDR2, and HCDR3, respectively.
EVQLLESGGGLVQPGGSLRLSCATSGFTFSSYAMTWVRQAPGKGLEWVSAISGGGATTYSTDSVKGRFTTSRDNSKNTLY
LQMNNLRAEDTALYYCAKGAFYNILSSIIVEGDYWGQGTLVTVSS
SEQ ID NO: 117 is the amino acid sequence of the COV49-04 V_{L}, SEQ ID NOs: 118, 119, and 120
are the amino acid sequences of the LCDR1, LCDR2, and LCDR3, respectively.
DVVMTQSPLSLSVTLGQPASISCRSSQSLVFSDGDTYLNWFQQRPGQSPRRLIYRVSNRDSGVPDRFSGSGSGTDFTLKI
SRVEAEDVGVYYCMQGTHWLSITFGQGTRLDIK
p) SEQ ID NO: 121 is the amino acid sequence of the COV49-05 V_{H}, SEQ ID NOs: 122, 123, and
124 are the amino acid sequences of the HCDR1, HCDR2, and HCDR3, respectively.
EVQLLESGGGLAQPGGSLRLSCVASGFSFNDYAMTWVRQAPGKGLEWVSAVSSGGQTTYSTDSVKGRFTISRDNSKNMLY
LQMNSLRAEDTAVYYCVKGAFYNILSSIIVEGDYWGQGTLVTVSS
SEQ ID NO: 125 is the amino acid sequence of the COV49-05 V_{L}, SEQ ID NOs: 126, 127, and 128
are the amino acid sequences of the LCDR1, LCDR2, and LCDR3, respectively.
GVVLTQSPLSLPVTLGQPASISCRSSQSLVFSDGNTYLNWFQQRPGQSPRRLIYQVSNRDPGVPDRFSGSGSGTDFTLKI
SRVEAEDVGVYYCMQGTHWLSITFGQGTRLEIK
q) SEQ ID NO: 129 is the amino acid sequence of the COV49-06 V_{H}, SEQ ID NOs: 130, 131, and
132 are the amino acid sequences of the HCDR1, HCDR2, and HCDR3, respectively.
EVQLLESGGGLVRPGGSLRLSCAASGFTFTNYAMSWVRQAPGKGLEWVSAISGGDGRTSYYADSVKGRFTSSRDNSKNTL
YLQMNSLRAEDTAVYYCAKGAFYNILSSIIVEGDYWGQGTLVTVSS
SEQ ID NO: 133 is the amino acid sequence of the COV49-06 V_{L}, SEQ ID NOs: 134, 135, and 136
are the amino acid sequences of the LCDR1, LCDR2, and LCDR3, respectively.
DVVMTQSPLSLPVTLGQPASISCRSSQSLVFSDGNTYLNWFQQRPGQSPRRLIYKVSNRDSGVPDRFSGSGSGTDFTLKI
SRVEAEDVGVYYCMQGTHWLSITFGQGTRLEIR
r) SEQ ID NO: 137 is the amino acid sequence of the COV49-07 V_{H}, SEQ ID NOs: 138, 139, and
140 are the amino acid sequences of the HCDR1, HCDR2, and HCDR3, respectively.
EVQLLESGGGLVQPGGSLRVSCTVSGFTFSDYSMSWVRQAPGKGLEWVSAISGTGETAYYADFVKGRFTSSRDNSKNTLY
LQMNTLRGEDTAVYYCVKGAFYNILSSIIVEGDYWGQGTLVTVSS
SEQ ID NO: 141 is the amino acid sequence of the COV49-07 V_{L}, SEQ ID NOs: 142, 143, and 144
are the amino acid sequences of the LCDR1, LCDR2, and LCDR3, respectively.
GVVMTQSPLSLPVTLGQPASISCRSSQSLVFSDGNTYLNWFQQRPGQSPRRLIYKVSERDSGVPDRFSGSGSGTDFTLTI
SRVEAEDVGVYYCMQGTHWLSITFGQGTRLEIK
s) SEQ ID NO: 145 is the amino acid sequence of the COV49-18 V_{H}, SEQ ID NOs: 146, 147, and
148 are the amino acid sequences of the HCDR1, HCDR2, and HCDR3, respectively.
EVQLLESGGGLVQPGGSLRVSCAVSGFTFSNYAMSWVRQAPGKGLDWVSAISGSGETRYYADFVKGRFTISRDNSKNTLY
LQMNSLRGEDTAVYYCVKGAFYNILSSIIVEGDYWGQGTLVTVSS
SEQ ID NO: 149 is the amino acid sequence of the COV49-18 V_{L}, SEQ ID NOs: 150, 151, and 152
are the amino acid sequences of the LCDR1, LCDR2, and LCDR3, respectively.
DVVMTQSPLSLPVTLGQPASISCRSSQSLVFSDGNTYLNWFQQRPGQSPRRLIYKVSKRDSGVPDRFSGSGSGTDFTLKI
SRVEAEDVGVYYCMQGTHWLSITFGQGTRLEIK
t) SEQ ID NO: 153 is the amino acid sequence of the COV49-23 V_{H}, SEQ ID NOs: 154, 155, and
156 are the amino acid sequences of the HCDR1, HCDR2, and HCDR3, respectively.
EVQLLESGGGLAQPGGSLRISCVASGFSFNDYAMAWVRRAPGKGLEWVSAVSGGGQTTYSTDSVKGRFTTSRDNSKNTLY
LQMNGLRAEDTAVYYCVKGAFYNILSSIIVEGDYWGQGALVTVSS
SEQ ID NO: 157 is the amino acid sequence of the COV49-23 V_{L}, SEQ ID NOs: 158, 159, and 160
aretheaminoacidsequencesoftheLCDR1,LCDR2,andLCDR3,respectively.
GVVLTQSPLSLPVTLGQPASISCRSSQSLVFSDGNTYLNWFQQRPGQSPRRLIYQVSNRDPGVPDRFSGSGSGTDFTLKI
SRVEAEDVGVYYCMQGTHWLSITFGQGTRLEIK
u) SEQ ID NO: 161 is the amino acid sequence of the COV49-28 V_{H}, SEQ ID NOs: 162, 163, and
164 are the amino acid sequences of the HCDR1, HCDR2, and HCDR3, respectively.
GVQLLESGGGLAQPGGSLKLSCVASGFSFNDYAMTWVRRPPGKGLEWVSAVSSGGQTSYSTDSVKGRFTISRDNSKNTLY
LQMSSLRAEDTAIYYCVKGAFYNILSSIIVEGDYWGQGTLVTVSS
SEQ ID NO: 165 is the amino acid sequence of the COV49-28 V_{L}, SEQ ID NOs: 166, 167, and 168
are the amino acid sequences of the LCDR1, LCDR2, and LCDR3, respectively.
GVVLTQSPLSLPVTLGQPASISCRSSQSLVFSDGKTYLNWFQQRPGQSPRRLIYQVSNRDPGVPDRFSGSGSGTDFTLKI
SRVEAEDVGVYYCMQGTHWLSITFGQGTRLEIK
v) SEQ ID NO: 169 is the amino acid sequence of the COV49-30 V_{H}, SEQ ID NOs: 170, 171, and
172 are the amino acid sequences of the HCDR1, HCDR2, and HCDR3, respectively.
EVQLLESGGGLVQPGGSLRVSCTVSGFTFSDYSMSWVRQAPGKGLDWVSAISGTGETAYYADFVKGRFTSSRDNSKNTVY
LQMNSLRGEDTAVYYCVKGVFYNILSSIIVEGDYWGQGTLVTVSS
SEQ ID NO: 173 is the amino acid sequence of the COV49-30 V_{L}, SEQ ID NOs: 174, 175, and 176
are the amino acid sequences of the LCDR1, LCDR2, and LCDR3, respectively.
GVVMTQSPLSLPVTLGQPASISCRSSQSLVFSDGNTYLNWFQQRPGQSPRRLIYKVSKRDSGVPDRFSGSGSGTDFTLTI
SRVEAEDVGVYYCMQGTHWLSITFGQGTRLEIK
w) SEQ ID NO: 177 is the amino acid sequence of the COV49-33 V_{H}, SEQ ID NOs: 178, 179, and
180 are the amino acid sequences of the HCDR1, HCDR2, and HCDR3, respectively.
EVQLLESGGGLAQPGGSLRLSCVASGFSFDDYAMTWVRQPPGKGLEWVSAVSGGGQTTYSTDSVKGRFTTSRDNSKNTLY
LQMNSLRGEDTAVYYCVKGAFYNILSSIIVEGDYWGQGTLVTVSS
SEQ ID NO: 181 is the amino acid sequence of the COV49-33 V_{L}, SEQ ID NOs: 182, 183, and 184
are the amino acid sequences of the LCDR1, LCDR2, and LCDR3, respectively.
VVVLTQSPLSLPVTLGQPASVSCRSSQSLVFSDGNTYLNWFQQRPGQSPRRLIYQVSNRDPGVPDRFSGSGSGTDFTLKI
SRVEAEDVGVYYCMQGTHWLSITFGQGTRLEIK
x) SEQ ID NO: 185 is the amino acid sequence of the COV49-42 V_{H}, SEQ ID NOs: 186, 187, and
188 are the amino acid sequences of the HCDR1, HCDR2, and HCDR3, respectively.
QVHLVQSGAEVKNPGVSVKVSCKASGYTFTTSGITWVRQAPGQGLEWMGWISTYNGNTETAQTFQGRVTMTTDTSTSTAY
MELRRLRSDDTAVYYCARVQSVATSSNYFFDYWGQGTRVTVSS
SEQ ID NO: 189 is the amino acid sequence of the COV49-42 V_{L}, SEQ ID NOs: 190, 191, and 192
are the amino acid sequences of the LCDR1, LCDR2, and LCDR3, respectively.
DIQMTQSPSSLSASVGDRVTITCRASQTIDRFLNWYQQKPGKAPKLLIYAASRLQSGVPSRFSGSGSGTTYTLTISSLQP
EDFATYFCQQSYSDPRALTFGGGTKVEIK
y) SEQ ID NO: 193 is the amino acid sequence of the COV49-47 V_{H}, SEQ ID NOs: 194, 195, and
196 are the amino acid sequences of the HCDR1, HCDR2, and HCDR3, respectively.
EVQLLESGGGLAQPGGSLRLSCVASGFSFNDYAMAWVRQAPGKGLEWVSAVSGGGQTTYSTDSVKGRFTTSRDNSKNTLY
LQMNGLRAEDAAVYYCVKGAFYNILSSIIVEGDNWGQGTLVTVSS
SEQ ID NO: 197 is the amino acid sequence of the COV49-47 V_{L}, SEQ ID NOs: 198, 199, and 200
are the amino acid sequences of the LCDR1, LCDR2, and LCDR3, respectively.
GVVLTQSPLSLPVTLGQPASISCRSSQSLVFSDGNTYLNWFQQRPGQSPRRLIYQVSNRDPGVPDRFSGSGSGTDFTLKI
SRVEAEDVGVYYCMQGTHWLSITFGQGTRLEIK
z) SEQ ID NO: 201 is the amino acid sequence of the COV49-54 V_{H}, SEQ ID NOs: 202, 203, and
204 are the amino acid sequences of the HCDR1, HCDR2, and HCDR3, respectively.
EVQLVESGGGLVQPGGSLRLSCAASGFIVSSNYMSWVRQAPGKGLEWVSMIYSGGSTVYADSVKGRFTISRDISKNTLYL
QMNSLRAEDTAVYYCALETFDYWGQGTLVTVSS
SEQ ID NO: 205 is the amino acid sequence of the COV49-54 V_{L}, SEQ ID NOs: 206, 207, and 208
are the amino acid sequences of the LCDR1, LCDR2, and LCDR3, respectively.
DIQMTQSPSSLSASVGDRVTITCRASQGITNHLAWYQQKPGKVPKLLIYAASTLQSGVPSRFSGSGYGTDFTLTISSLQP
EDVASYYCQKYDSAPRTFGQGTKVEIK
aa) SEQ ID NO: 209 is the amino acid sequence of the COV57-01 V_{H}, SEQ ID NOs: 210, 211, and
212 are the amino acid sequences of the HCDR1, HCDR2, and HCDR3, respectively.
QVQLVESGGGVVQPGRSLRLSCTVSGFTFNNYGMHWVRQAPGKGLEWLAVISYEGSKKFYADSLEGRFSISRDSSKNTLY
LQMNSLTIEDTAVYFCAKRNSIFWFGEGRKDAFDVWGQGTRVIVSS
SEQ ID NO: 213 is the amino acid sequence of the COV57-01 V_{L}, SEQ ID NOs: 214, 215, and 216
are the amino acid sequences of the LCDR1, LCDR2, and LCDR3, respectively.
DIQLTQSPSSLSASVGDRVTITCRASQTVDNYLNWYQQLPGKAPKLLIHTASTLQSGVPSRFSATSSGTDFTLTISGLQP
DDSATYFCQQSYSVPLTFGGGTKVEIK
bb) SEQ ID NO: 217 is the amino acid sequence of the COV57-03 V_{H}, SEQ ID NOs: 218, 219, and
220 are the amino acid sequences of the HCDR1, HCDR2, and HCDR3, respectively.
QVHLVESGGGVVQPGRSLRLSCAASGFTFSSFGMHWVRQAPGKGLEWVAVISDDGNNKFYADSVKGRFSISRDNSKNTLY
LQMNSLRAEDTAVYYCVRAFRNSNWATGEYFDYWGQGTLVTVSS
SEQ ID NO: 221 is the amino acid sequence of the COV57-03 V_{L}, SEQ ID NOs: 222, 223, and 224
are the amino acid sequences of the LCDR1, LCDR2, and LCDR3, respectively.
DIVMTQSPDSLAVSLGERATINCKSSQSVLYSSNNKNYLAWYQQKPGQPPKLLIYWASTRESGVPDRFSGSGSGTDFTLT
ISSLQAEDVAVYYCQQFHRIPPTFGQGTKVEIK
cc) SEQ ID NO: 225 is the amino acid sequence of the COV57-04 V_{H}, SEQ ID NOs: 226, 227, and
228 are the amino acid sequences of the HCDR1, HCDR2, and HCDR3, respectively.
QVQLVESGGGVVQPGRSLRLSCAASGFTFSNFGMHWVRQAPGKGLEWVAVISDDGNNEFYAGSVKGRFSISRDNSKNTLY
LQMNSLRPEDTAVYYCVRAFRNSNWATGEYFDYWGQGTLVTVSS
SEQ ID NO: 229 is the amino acid sequence of the COV57-04 V_{L}, SEQ ID NOs: 230, 231, and 232
are the amino acid sequences of the LCDR1, LCDR2, and LCDR3, respectively.
DIVMTQSPDSLAVSLGERATINCKSSQSVLYSSNNKNYLAWYQQKPGQPPKLLIYWASTRESGVPDRFSGSGSGTDFTLT
ISSLQAEDVAVYYCQQFHRIPPTFGQGTKVEIK
dd) SEQ ID NO: 233 is the amino acid sequence of the COV57-05 V_{H}, SEQ ID NOs: 234, 235, and
236 are the amino acid sequences of the HCDR1, HCDR2, and HCDR3, respectively.
QVQLLESGPGLVKPSGTLSLTCDVSGDSISSSNWWSWVRQSPGKGLEWIGEIYQSGTTNNNPSLKSRVTISIDKSKNQFS
LNLTSVTAADTAVYYCARAPLVFRSYGMDVWGQGTTVTVSS
SEQ ID NO: 237 is the amino acid sequence of the COV57-05 V_{L}, SEQ ID NOs: 238, 239, and 240
are the amino acid sequences of the LCDR1, LCDR2, and LCDR3, respectively.
DIQMTQSPSSLSASVGDRVTITCRASQTIRTYLNWYQQKPGKAPKLLIYDASNLQSGVPSRFSGSGSGTDFTLTISSLEP
EDFATYYCQQSYITPPWTFGQGTRVEIK
ee) SEQ ID NO: 241 is the amino acid sequence of the COV57-13 V_{H}, SEQ ID NOs: 242, 243, and
244 are the amino acid sequences of the HCDR1, HCDR2, and HCDR3, respectively.
QVQLVQSGTEVKKPGSSVKVSCRASGGTLSSYSVSWVRQAPGQGLEWMGRIIPILDKKTYAQKYQGRVTIIADTSTSTVY
MELSSLKSEDSAVYYCTTFDYDTSGQRDQHDYWGQGTLVTVSS
SEQ ID NO: 245 is the amino acid sequence of the COV57-13 V_{L}, SEQ ID NOs: 246, 247, and 248
are the amino acid sequences of the LCDR1, LCDR2, and LCDR3, respectively.
DIVLTQSPDSLAVSLGERATINCKSSQSVLYRSSNQNYLTWYQQKPGQPPKLLIYWASTRESGVPDRFSGSGSGTDFTLT
ISSLQAEDVAVYFCQQFYTTPFTFGPGTRVDIK
ff) SEQ ID NO: 249 is the amino acid sequence of the COV57-19 V_{H}, SEQ ID NOs: 250, 251, and
252 are the amino acid sequences of the HCDR1, HCDR2, and HCDR3, respectively.
QVQLQESGPGLVKPSGTLSLTCAVSGDSISSSNWWSWVRQPPGKGLEWIGEIYQSGTTNNNPSLKSRVTISIDKSKNQFS
LNLTSVTAADTAVYYCARAPLVFRSYGMDVWGQGTTVTVSS
SEQ ID NO: 253 is the amino acid sequence of the COV57-19 V_{L}, SEQ ID NOs: 254, 255, and 256
are the amino acid sequences of the LCDR1, LCDR2, and LCDR3, respectively.
DIQMTQSPSSLSASVGDRVTITCRASQTIRTYLNWYQQKPGKAPKLLIYDASNLQSGVPSRFSGSGSGTDFTLTISSLEP
EDFATYYCQQSYITPPWTFGQGTKVEIK
gg) SEQ ID NO: 257 is the amino acid sequence of the COV57-34 V_{H}, SEQ ID NOs: 258, 259, and
260 are the amino acid sequences of the HCDR1, HCDR2, and HCDR3, respectively.
QVQLVQSGTEVKKPGSSVKVSCRASGGTLSSYSVSWVRQAPGQGLEWMGRIIPILDKKTYAQKYQGRVTIIADTSTSTVY
MELSSLKSDDSAVYYCTTFDYDTSGQRDQHDYWGQGTPVTVSS
SEQ ID NO: 261 is the amino acid sequence of the COV57-34 V_{L}, SEQ ID NOs: 262, 263, and 264
are the amino acid sequences of the LCDR1, LCDR2, and LCDR3, respectively.
DIVLTQSPDSLAVSLGERATINCKSSQSVLYSSSNQNYLTWYQQKPGQPPKLLIFWASTRESGVPDRFSGSGSGTDETLT
ISSLQPEDVAVYFCHQFYSTPFTFGPGTRVDIK
hh) SEQ ID NO: 265 is the amino acid sequence of the COV57-38 V_{H}, SEQ ID NOs: 266, 267, and
268 are the amino acid sequences of the HCDR1, HCDR2, and HCDR3, respectively.
QVQLVESGGGVVQPGRSLRLSCEASGFNFNNYGMHWVRQAPGKGLEWVAVISYEGSKKYYEDSVRGRFTISRDFSKNTLY
LQMNSLRAEDTAVYYCAKANEIFWLGQFKNDALDFWGQGTMVTVSS
SEQ ID NO: 269 is the amino acid sequence of the COV57-38 V_{L}, SEQ ID NOs: 270, 271, and 272
are the amino acid sequences of the LCDR1, LCDR2, and LCDR3, respectively.
QSVLTQPPSASGTPGQRVAVSCSGSSSNIGTNFVNWYQHLPRTAPKLLIYRDNQRPSGVPDRFSGSKSGTSASLAISGLQ
SEDEADYYCAVWDDSLNGQVFGGGTKVTVL
ii) SEQ ID NO: 273 is the amino acid sequence of the COV57-45 V_{H}, SEQ ID NOs: 274, 275, and
276 are the amino acid sequences of the HCDR1, HCDR2, and HCDR3, respectively.
QVQLVQSGTEVKKPGSSVKVSCRASGGTLSSYSVSWVRQAPGQGLEWMGRIIPILDKKTYAQKYQGRVTILADTSTSTVY
MELSSLKSDDSAVYFCTTFDYDTSGQRDQHDYWGQGTPVTVSS
SEQ ID NO: 277 is the amino acid sequence of the COV57-45 V_{L}, SEQ ID NOs: 278, 279, and 280
are the amino acid sequences of the LCDR1, LCDR2, and LCDR3, respectively.
DIVLTQSPDSLAVSLGERATINCKSSQSVLYSSSNQNYLTWYQQKPGQPPKLLIFWASTRESGVPDRFSGSGSGTDETLT
ISSLQPEDVAVYFCHQFYSTPFTFGPGTRVDIK
jj) SEQ ID NO: 281 is the amino acid sequence of the COV77-02 V_{H}, SEQ ID NOs: 282, 283, and
284 are the amino acid sequences of the HCDR1, HCDR2, and HCDR3, respectively.
QVQLVESGGGVVQPGRSLRLSCAASGFTFSDHGMHWVRQAPGKGLQWVALIWYDGSNMFYRDSVKGRFTISRDNSKNTVY
LEMSSLRAEDTALYYCARGTDVWSGLSLDALDVWGQGTMVTVSP
SEQ ID NO: 285 is the amino acid sequence of the COV77-02 V_{L}, SEQ ID NOs: 286, 287, and 288
are the amino acid sequences of the LCDR1, LCDR2, and LCDR3, respectively.
DIVMTQSPLSLPVTPGEPASISCRSSQSLLHYNGYNYLDWYLQKPGQSPRLLIYLGSNRASGVPDRFSGSGSGTDFTLKI
SRVEAEDVGVYYCMQALQLNLITFGQGTRLEIK
kk) SEQ ID NO: 289 is the amino acid sequence of the COV77-04 V_{H}, SEQ ID NOs: 290, 291, and
292 are the amino acid sequences of the HCDR1, HCDR2, and HCDR3, respectively.
QVRLQESGPGLVKPSETLSVTCTVSGVSISNYYWSWIRQPPGKGLEWIGYISDTGSTNYSPSLQSRVTISVDMSKNQVSL
EVTSVTATDTAVYYCARTTTSVPFDYWGKGVLVTVSS
SEQ ID NO: 293 is the amino acid sequence of the COV77-04 V_{L}, SEQ ID NOs: 294, 295, and 296
are the amino acid sequences of the LCDR1, LCDR2, and LCDR3, respectively.
SYELTQPPSVSVSPGQVARITCSGEALPKKYAYWYQHKPGQFPLLVIHKDSERPSGIPERFSGSSSGTVVILTISGVQAE
DEADYFCLSTDGSTTWVFGGGTKLTVL
ll) SEQ ID NO: 297 is the amino acid sequence of the COV77-05 V_{H}, SEQ ID NOs: 298, 299, and
300 are the amino acid sequences of the HCDR1, HCDR2, and HCDR3, respectively.
QMQLHESGPGLVKPSQTLSLTCAVSGGSISSDGYYWNWVRQHPGKGLELIAYIYYNGRPYYNPSLRSRVNISLDTSMNQF
SLRMKSLTAADTAVYYCARGRLGYCAGGSCFLESFDYWGQGALVTVSS
SEQ ID NO: 301 is the amino acid sequence of the COV77-05 V_{L}, SEQ ID NOs: 302, 303, and 304
are the amino acid sequences of the LCDR1, LCDR2, and LCDR3, respectively.
EIVMTQSPATLSVSPGETATLSCRASESISSNLAWYQQKPGQAPRLLIYGASTRATGIPARFSGSGSGTEFALTISSLQA
EDFAVYSCQQYHNWLLTFGGGTKVEIK
mm) SEQ ID NO: 305 is the amino acid sequence of the COV77-09 V_{H}, SEQ ID NOs: 306, 307,
and 308 are the amino acid sequences of the HCDR1, HCDR2, and HCDR3, respectively.
EVQLVESGGGLVQPGRSLTLSCSGSGFTFGDYSMSWVRQAPGKGLEWLGFIRSQTYGGTTQYAASVQGRFTILRDDSKSI
AYLQMNSLKTEDTAVYYCTRNYGVGDYWGQGTLVTVSS
SEQ ID NO: 309 is the amino acid sequence of the COV77-09 V_{L}, SEQ ID NOs: 310, 311, and 312
are the amino acid sequences of the LCDR1, LCDR2, and LCDR3, respectively.
DIVMTQSPASLAVSLGERATIHCRSSLSVLNSSNNKNYLAWYQQKAGQPPKLLMYWASTRESGVPERFSGSGSGTDETLT
ISSLEAGDVAVYYCQQYYDKLSLTFGGGTKVEIK
nn) SEQ ID NO: 313 is the amino acid sequence of the COV77-14 V_{H}, SEQ ID NOs: 314,
315, and 316 are the amino acid sequences of the HCDR1, HCDR2, and HCDR3, respectively.
QVQLQESGPGLVKASQTLSLTCAVSGGSISSGDYYWTWIRSHPGKGLEWIAYIYYNGRTYYNPSLKSRLTISIDRSKNEF
SLKVRSVTAADSAAYYCARGRLGFCAAGSCFLDSLDFWGQGTLVTVSS
SEQ ID NO: 317 is the amino acid sequence of the COV77-14 V_{L}, SEQ ID NOs: 318, 319, and 320
are the amino acid sequences of the LCDR1, LCDR2, and LCDR3, respectively.
EIVMTQSPATLSVSPGERVTLSCRASESISNNLAWYQQKPGQAPRLLIYGASTRATGIPARFSGSGSGTEFALIISSLQS
EDFAVYFCQQYYNWLLTFGGGTKVEIK
oo) SEQ ID NO: 321 is the amino acid sequence of the COV77-35 V_{H}, SEQ ID NOs: 322, 323, and
324 are the amino acid sequences of the HCDR1, HCDR2, and HCDR3, respectively.
QMQLQESGPGLVKPSQTLSLTCAVSGGSISSDGYYWNWVRQHPGKGLELIAYIYYNGRPYYNPSLRSRVNISLDTSMNQF
SLRVKSLTAADTAVYYCARGRLGYCAAGSCFLDSFDYWGQGALVTVSS
SEQ ID NO: 325 is the amino acid sequence of the COV77-35 V_{L}, SEQ ID NOs: 326, 327, and 328
are the amino acid sequences of the LCDR1, LCDR2, and LCDR3, respectively.
EIVMTQYPATLSVSPGETATLSCRASESISRNLAWYQQKPGQAPRLLIYGASTRATGIPARFSGSGSGTEFALTISSLQA
EDFAVYSCQQYHNWLLTFGGGTKVEIK
pp) SEQ ID NO: 329 is the amino acid sequence of the COV77-39 V_{H}, SEQ ID NOs: 330, 331, and
332 are the amino acid sequences of the HCDR1, HCDR2, and HCDR3, respectively.
RVQLQESGPGLVKPSETLSLTCTVSGGSITDDYWNWIRQPPGKGLEWIGYVHDSGTTNYNPSLKSRVTLSLHTSGNKFSL
RLTSVTAADTAMYYCARTTTTFPFDSWGQGTLVIVSS
SEQ ID NO: 333 is the amino acid sequence of the COV77-39 V_{L}, SEQ ID NOs: 334, 335, and 336
are the amino acid sequences of the LCDR1, LCDR2, and LCDR3, respectively.
SYELTQPPSVSVSLGQMARITCSGEALPKKYVYWYQQKPGQFPVLVIYKDRERPSGIPERFSGSSSGTIVILTISGVQAE
DEADYYCLSVDSTANWVFGGGTKLTVL
qq) SEQ ID NO: 337 is the amino acid sequence of the COV77-42 V_{H}, SEQ ID NOs: 338, 339, and
340 are the amino acid sequences of the HCDR1, HCDR2, and HCDR3, respectively.
QVYLQESGPGLVKPSQTLSLTCAVSGGSIRSDGYYWTWIRQHPGKGLEWIGNTYYNGNTYYNPSLRSRVTTSLDTSKNQF
SLKVVSMTGADTAVYYCARGRLGFCAGGACFLDTLDYWGQGTLVTVSS
SEQ ID NO: 341 is the amino acid sequence of the COV77-42 V_{L}, SEQ ID NOs: 342, 343, and 344
are the amino acid sequences of the LCDR1, LCDR2, and LCDR3, respectively.
EIVMTQSPVTLSVSPGERVTLSCRADQSVNNNLAWYQQKPGQAPRLLIHGASTRATGIPARFSGSGSGTEFTLTISSLQS
EDFAIYYCQQYYNWLLTFGGGTKLEIK
rr) SEQ ID NO: 345 is the amino acid sequence of the COV77-43 V_{H}, SEQ ID NOs: 346, 347, and
348 are the amino acid sequences of the HCDR1, HCDR2, and HCDR3, respectively.
QVQLVESGGGVVQPGRSLRLSCSASGFTFSDYGMNWVRQAPGKGLEWVAMIWYDGSNYFYRDSVKGRFAISRDNSKNMVF
LQMDNLRAEDTAVYFCARGTHFWSGLSLDAFDMWGQGTVVTVSS
SEQ ID NO: 349 is the amino acid sequence of the COV77-43 V_{L}, SEQ ID NOs: 350, 351, and 352
are the amino acid sequences of the LCDR1, LCDR2, and LCDR3, respectively.
EIVMTQSPLSLPVTPGEPASISCRSSRSLLHYNGHNYLDWYLQKPGQSPQLLIYLGSNRAPGVPDRFSGSGSGTDFTLKI
SRVEAEDVGVYYCMQALQLNLITFGQGTRLEIK
ss) SEQ ID NO: 353 is the amino acid sequence of the COV77-46 V_{H}, SEQ ID NOs: 354, 355, and
356 are the amino acid sequences of the HCDR1, HCDR2, and HCDR3, respectively.
QVQLQESGPGLVKPSQPLSLTCAVSGDSISSGGNYWSWIRQHPGKGLECIAYFYYTGRTYYNPSLKSRVTISLDTSKNQF
SLKVSSVTAADTAVYFCARGRLGHCGGGSCFLDPLDYWGQGSLVTVSS
SEQ ID NO: 357 is the amino acid sequence of the COV77-46 V_{L}, SEQ ID NOs: 358, 359, and 360
are the amino acid sequences of the LCDR1, LCDR2, and LCDR3, respectively.
EIVMTQSPATLSVSPGERATLSCRASERISSNLAWSQHKVGQAPRLLIYGASTRATGIPARFSGSGSGTEFTLTISSLQS
EDFAVYYCHQYHNWLLTFGGGTKVEIK
tt) SEQ ID NO: 361 is the amino acid sequence of the COV77-76 V_{H}, SEQ ID NOs: 362, 363, and
364 are the amino acid sequences of the HCDR1, HCDR2, and HCDR3, respectively.
QVQLQESGPGLVKPSETLSLTCTVSGGSISSFYWSWIRQPPGKRLEWIGYIYYRGSTNYNPSLKSRVTMSVDTSKNQFSL
KLSSVTAADTAVYYCARELPNNWLDPWGQGTLVTVSS
SEQ ID NO: 365 is the amino acid sequence of the COV77-76 V_{L}, SEQ ID NOs: 366, 367, and 368
are the amino acid sequences of the LCDR1, LCDR2, and LCDR3, respectively.
DIVMTQSPDSLAVSLGERATINCKSSQSILYNSNNKNYLAWYQQKPGQPPKLLIYWASTRESGVPDRFSGSGSGTDFTLT
ISSLQAEDVAVYYCQQYHLTPWTFGQGTKVEVT
uu) SEQ ID NO: 369 is the amino acid sequence of the COV93-04 V_{H}, SEQ ID NOs: 370, 371, and
372 are the amino acid sequences of the HCDR1, HCDR2, and HCDR3, respectively.
QLQLQESGSGLVKPSQTLSLTCVVSGDSISSGGYSWSWIRQQPGKALEWIGYIYHSGVTNYNPSLKSRVTISVDRSTNQF
SLKLSSVTAADTAVYYCARDGKYNWFDPWGQGILVTVSS
SEQ ID NO: 373 is the amino acid sequence of the COV93-04 V_{L}, SEQ ID NOs: 374, 375, and 376
are the amino acid sequences of the LCDR1, LCDR2, and LCDR3, respectively.
DIVMTQSPDSLAVSLGERAVINCKSSQNVLYTSNNKNYLAWYQQKAGRPPKLLIYWASTRESGVPDRFSGSGSGTDETLT
ISSLQAEDVAVYYCQQYFDTPWTFGQGTKVEIK
vv) SEQ ID NO: 377 is the amino acid sequence of the COV93-08 V_{H}, SEQ ID NOs: 378, 379, and
380 are the amino acid sequences of the HCDR1, HCDR2, and HCDR3, respectively.
QVQLRESGPGVVKPSGTLSLTCTVSGDFINNNRWWTWVRQSPGKGLEWIGEIYHSGSTNYNPSLKGRVTISVDKSKNHFS
LKLTSVTAADTAMYYCARGGYCTVGDCHDNWFDPWGQGTLVTVSS
SEQ ID NO: 381 is the amino acid sequence of the COV93-08 V_{L}, SEQ ID NOs: 382, 383, and 384
are the amino acid sequences of the LCDR1, LCDR2, and LCDR3, respectively.
DIQMTQSPSTLSASVGDRVTITCRASQNINSWLAWYQQKPGKAPKVLIYKASSLESGVPSRFSGSVSGTEFTLTISSLQP
DDFATYYCQQYSTLSWTFGQGTKVDIK
ww) SEQ ID NO: 385 is the amino acid sequence of the COV93-17 V_{H}, SEQ ID NOs: 386, 387,
and 388 are the amino acid sequences of the HCDR1, HCDR2, and HCDR3, respectively.
EVQLVQSGAEVKKPGESLRISCRGSGYSFTNYWISWVRQMPGKGLEWMGRIDPTDSYTNYSPSFQGHVTISTDKSISTAY
LQWSSLKASDTAMYYCARHKNGMIHEYDYWGQGILVTVSS
SEQ ID NO: 389 is the amino acid sequence of the COV93-17 V_{L}, SEQ ID NOs: 390, 391, and 392
are the amino acid sequences of the LCDR1, LCDR2, and LCDR3, respectively.
DIQLTQSPSTLSASVGDRVTITCRASQSIRSWLAWYQQKPGKAPKLLIYRASSLESGVPSRFSGSGSGTEFTLTISSLQP
DDFATYYCQQYGTFGPGTKVDIR
xx) SEQ ID NO: 393 is the amino acid sequence of the COV93-18 V_{H}, SEQ ID NOs: 394, 395, and
396 are the amino acid sequences of the HCDR1, HCDR2, and HCDR3, respectively.
RVHLVESGGGVVQPGRSLRLSCATSGFTFSNYAIHWVRQAPGKGLEWVAAVSDDGNSIFYAQSVKGRFTTSRDNSKSTLY
LQMNGLRSEDTAVYYCARAQYSDSWGLGNWIDTWGQGTLVTVSS
SEQ ID NO: 397 is the amino acid sequence of the COV93-18 V_{L}, SEQ ID NOs: 398, 399, and 400
are the amino acid sequences of the LCDR1, LCDR2, and LCDR3, respectively.
DIVMTQSPDSLAVSLGERTTINCKSSQSVLYSSNNKNYLAWYQQKPGQAPKVLLYWASTRESGVPDRFSGSGSGTDENLT
ISSLQAEDVAVYYCHQYYSPPQTFGQGTKVEIK
yy) SEQ ID NO: 401 is the amino acid sequence of the COV93-23 V_{H}, SEQ ID NOs: 402, 403, and
404 are the amino acid sequences of the HCDR1, HCDR2, and HCDR3, respectively.
QVQVVESGGGVVQPGGSLRLSCATSGFTFSSYAIHWVRQAPGKGLEWVAAVSDDGNSIFYAQSVKGRESTSRDNSKNTLY
LQMNGLRTEDTAIYYCARADYSDSWGLGNWIDTWGQGTLVTVSS
SEQ ID NO: 405 is the amino acid sequence of the COV93-23 V_{L}, SEQ ID NOs: 406, 407, and 408
are the amino acid sequences of the LCDR1, LCDR2, and LCDR3, respectively.
DIVMTQSPDSLAVSLGERATIYCKSSQSVLYSSNNKNYLAWYQQKPGQPPKVLIYWASTRESGVPDRFTGSGSGTDFSLT
ISSLHPEDVAVYYCHQYYSPPQTFGQGTKVEIK
zz) SEQ ID NO: 409 is the amino acid sequence of the COV78-36 V_{H}, SEQ ID NOs: 410, 411, and
412 are the amino acid sequences of the HCDR1, HCDR2, and HCDR3, respectively.
EVQLVQSGAEVKKPGESLRISCKGSGYSFSNSWITWVRQMPGKGLEWVGRIDTRDSRTAYSPSFEGHVFFSADRSSDTVY
VQWRSLRASDTAVYYCTTGAGVVIAASYFWGQGTLVTVSS
SEQ ID NO: 413 is the amino acid sequence of the COV78-36 V_{L}, SEQ ID NOs: 414, 415, and 416
are the amino acid sequences of the LCDR1, LCDR2, and LCDR3, respectively.
QSALTQPPSASGSPGQPVTISCTGTSSDVGGYEYVSWYQQLPGKVPTLIIHEVSKRPSGVPDRFSGSKSGNTASLTVSGL
QAEDEADYYCSSYSADSSIIFGGGTKLTVL
aaa) SEQ ID NO: 417 is the amino acid sequence of the COV93-38 V_{H}, SEQ ID NOs: 418, 419,
and 420 are the amino acid sequences of the HCDR1, HCDR2, and HCDR3, respectively.
QVQLQESGPGVVKPSGTLSLTCAVSGDFSNNNRWWTWVRQSPGKGLEWIGEIHQSGSTNYNPSLKSRITISVDKSKNQFS
LKLTSVTAADTAVYYCARGGYCSVGDCHDNWFDPWGQGTLVTVSS
SEQ ID NO: 421 is the amino acid sequence of the COV93-38 V_{L}, SEQ ID NOs: 422, 423, and 424
are the amino acid sequences of the LCDR1, LCDR2, and LCDR3, respectively.
DIQMTQSPSTLSASVGDRVTITCRASQTINSWLAWYQQKPGKAPKVLIYKASSLESGVPSRFSGSVSGTEFTLTISSLQP
DDFATYYCQQYSSLSWTFGQGTKVDIK
bbb) SEQ ID NO: 425 is the amino acid sequence of the COV93-60 V_{H}, SEQ ID NOs: 426, 427,
and 428 are the amino acid sequences of the HCDR1, HCDR2, and HCDR3, respectively.
QVQLQESGPGLVKPSETLFLTCAVSGGSINSSYWSWIRQPPGKGLEWIGYIYYRGSTNYNPSLKSRVTISVDTSKNQFSL
KLNSVTAADTAIYYCARENLWNWFDPWGQGILVTVSS
SEQ ID NO: 429 is the amino acid sequence of the COV93-60 V_{L}, SEQ ID NOs: 430, 431, and 432
are the amino acid sequences of the LCDR1, LCDR2, and LCDR3, respectively.
DIVMTQSPDSLAVSLGERATINCKSSQSVLYSSNNKNYLAWYQQKPGQPPKLLIYWASIRESGVPDRFSGSGSGTDFTLT
ISGLQAEDVAVYYCQQYHNTPWTFGQGTKVEIK
ccc) SEQ ID NO: 433 is the amino acid sequence of the COV93-61 V_{H}, SEQ ID NOs: 434, 435,
and 436 are the amino acid sequences of the HCDR1, HCDR2, and HCDR3, respectively.
EVQLVQSGAEVKKPGESLKISCKGSGYSFTTFWIGWVRQMPGKGLEWVGVIYPDDSDTRYSPSFQGQVTMSADKSINTAY
LQWSSLKASDTAVYYCARRNCDAALCYLGTYDYWGQGTVVTVSS
SEQ ID NO: 437 is the amino acid sequence of the COV93-61 V_{L}, SEQ ID NOs: 438, 439, and 440
are the amino acid sequences of the LCDR1, LCDR2, and LCDR3, respectively.
QSALTQPPSASGSPGQSVTISCTGTISDVGRYDFVSWYQHHPGKAPKLIIYEVTKRPSGVPDRFSGSKSGNTASLTVSGL
QAEDEADYYCNSYAGHNNFVIFGGGTKLTVL
ddd) SEQ ID NO: 441 is the amino acid sequence of the COV89-03 V_{H}, SEQ ID NOs: 442, 443,
and 444 are the amino acid sequences of the HCDR1, HCDR2, and HCDR3, respectively.
QVQLQESGPGLVKPSETLSLTCTVSGGSIRSNYWSWIRQPPGKRLEWLGYIYYSGSTNYNPSLKSRVTISVDTSKNQFSL
NLSSVTAADTAVYYCVREMPYNWIDPWGQGTLVTVSS
SEQ ID NO: 445 is the amino acid sequence of the COV89-03 V_{L}, SEQ ID NOs: 446, 447, and 448
are the amino acid sequences of the LCDR1, LCDR2, and LCDR3, respectively.
DIVMTQSPDSLAVSLGERATINCKSSQSISYSSNNKNYLAWYQQKPGQPPKLLIYWASTRESGVPDRFSGSGSGTDFTLT
ISSLQAEDVAVYFCQQYFDTPWTFGQGTKVEIK
eee) SEQ ID NO: 449 is the amino acid sequence of the COV89-28 V_{H}, SEQ ID NOs: 450, 451,
and 452 are the amino acid sequences of the HCDR1, HCDR2, and HCDR3, respectively.
EVQLVESGGGLVQPGRSLRLSCAASGFTFDDYAMHWVRQAPGKGLEWVSSISWNSGSIVYADSVTGRCTITRDNAKNSLY
LQMNSLRPEDTALYYCAKGYSSSPVVYYMDVWGKGTTVTVSS
SEQ ID NO: 453 is the amino acid sequence of the COV89-28 V_{L}, SEQ ID NOs: 454, 455, and 456
are the amino acid sequences of the LCDR1, LCDR2, and LCDR3, respectively.
DIQMTQSPSSVSASVGDRVTITCRASQGIAGWFAWYQQKPGKAPKLLIYATSSLQRGVPSRFSGSGSATHFTLTITSLQP
EDFATYYCQQANIFGPGTKVDIK
fff) SEQ ID NO: 457 is the amino acid sequence of the COV30-35 V_{H}, SEQ ID NOs: 458, 459, and
460 are the amino acid sequences of the HCDR1, HCDR2, and HCDR3, respectively.
QVQLQESGPGQVKPSGTLSLTCAVSGGSINSSNWWSWVRQPPGKGLEWIGEIFHSGNTNYKPSLESRVTISEDKSKKELS
LRLTSVTAADTAVYYCATLFCSGGTCYLGDWGQGTLVTVSS
SEQ ID NO: 461 is the amino acid sequence of the COV30-35 V_{L}, SEQ ID NOs: 462, 463, and 464
are the amino acid sequences of the LCDR1, LCDR2, and LCDR3, respectively.
EIVLTQSPGTLSVSPGQRATLSCRASESVSSRYVAWYQQKPGQAPRLIIYGASSRATGIPDRFSGSGSGTEFILSISRLE
PEDSAVYYCQQYGSSPTFGGGTKVEIK
ggg) SEQ ID NO: 465 is the amino acid sequence of the COV30-80 V_{H}, SEQ ID NOs: 466, 467,
and 468 are the amino acid sequences of the HCDR1, HCDR2, and HCDR3, respectively.
QVQLVESGGGVVQPGRSLRLSCAASGFTFSSYAMHWVRQAPGKGLEWVAVISYDGNNAFYADSVKGRFTISRDNSKNTVY
VQMDSLRAEETAVYYCARAGYNSNWDLGDALDIWGQGTMVTVSS
SEQ ID NO: 469 is the amino acid sequence of the COV30-80 V_{L}, SEQ ID NOs: 470, 471, and 472
are the amino acid sequences of the LCDR1, LCDR2, and LCDR3, respectively.
DIVMTQSPDSLAVSLGERATINCKSSQSVLYSSNNKNYLAWYQQKPGQPPKLLINWASTRESGVPDRFSGSGSGTDFTLT
ISSLQAEDVAVYYCQQYYSIPQTFGQGTKVEIK
hhh) SEQ ID NO: 473 is the amino acid sequence of the COV44-25 V_{H}, SEQ ID NOs: 474, 475,
and 476 are the amino acid sequences of the HCDR1, HCDR2, and HCDR3, respectively.
QVQLVDSGGGAVQPGTSLRLSCAASGFTFNTYAFHWVRQAPGKGLEWVAVISDDGNNKYYADSVKGRFTISRDDSKNTLS
LQMDSLRVEDAAVYFCARGSYNSNWSLGEYFDYWGQGSLVTVSS
SEQ ID NO: 477 is the amino acid sequence of the COV44-25 V_{L}, SEQ ID NOs: 478, 479, and 480
are the amino acid sequences of the LCDR1, LCDR2, and LCDR3, respectively.
DIVMTQSPDSLPVSLGERATINCKSSQSVLHSSNNKNYLAWYQQKPGQSPKLLISWASTRESGVPDRFSGSGSGTHFTLT
ISSLRAEDVAVYYCLQYFNNQYTFGQGTKLEIK
iii) SEQ ID NO: 481 is the amino acid sequence of the COV44-37 V_{H}, SEQ ID NOs: 482, 483, and
484 are the amino acid sequences of the HCDR1, HCDR2, and HCDR3, respectively.
QVQLVQSGAEVKKPGASVMLSCRASGYTFINDYMHWVRQAPGEGFEWMGIINPTGDGTKFAQKFQGRVTMTRDSSTTTVH
MELRSLTSGDTAVYFCATGGRQSAFDIWGQGTVVIVSS
SEQ ID NO: 485 is the amino acid sequence of the COV44-37 V_{L}, SEQ ID NOs: 486, 487, and 488
are the amino acid sequences of the LCDR1, LCDR2, and LCDR3, respectively.
DIVMTQSPLSLPVTPGESASISCRSSQSLLQNNGYNYLDWYLQKPGQSPQLLIYLGSYRASGVPDRFSGSGSGTDFTLKI
SRVEAEDVGVYYCVQAQQAPPLYTFGQGTKLQIK
SEQ ID NOs: 489 and 490 are the amino acid sequence of the stem helix in the S2 domain of the
spike protein of a coronavirus.
LQPELDSFKEELDKYFKNHTS
LDSFKEELDKYF
SEQ ID NO: 491 is the amino acid sequence of Peptide 42.
PSKPSKRSFIEDLLF
SEQ ID NO: 492 is the amino acid sequence of Peptide 43.
PSKRSFIEDLLFNKV
SEQ ID NO: 493 is the amino acid sequence of Peptide 44.
RSFIEDLLFNKVTLA
SEQ ID NO: 494 is the amino acid sequence of Peptide 153.
QPELDSFKEELDKYF
SEQ ID NO: 495 is the amino acid sequence of Peptide 154.
LDSFKEELDKYFKNH
SEQ ID NO: 496 is the amino acid sequence of Peptide 155.
FKEELDKYFKNHTSP
SEQ ID NO: 497 is the amino acid sequence of a fusion peptide site (partial).
SFIEDLLENKVTLA
SEQ ID NO: 498 is the amino acid sequence of an S2 peptide.
RSFIEDLLF
SEQ ID NOS: 499-532 are peptide sequences from coronavirus spike proteins, and are shown in
FIGS. 5C-5F.

### DETAILED DESCRIPTION OF SEVERAL EMBODIMENTS

Monoclonal antibodies, and antigen binding fragments thereof, that specifically bind the spike protein of SARS-CoV-2 and bind the spike protein of at least one additional coronavirus, such as an alphacoronavirus or a betacoronavirus, are disclosed herein. In some embodiments, the monoclonal antibody binds the S2 domain of the spike protein. Bispecific antibodies are also disclosed. These monoclonal antibodies and bispecific antibodies can be used to inhibit a coronavirus infection, such as, but not limited to, a SARS-Cov-2 infection. The monoclonal antibodies and bispecific antibodies also can be used to detect a coronavirus infection.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

About: Unless context indicated otherwise, “about” refers to plus or minus 5% of a reference value. For example, “about” 100 refers to 95 to 105.

Administration: The introduction of an agent, such as a disclosed antibody, into a subject by a chosen route. Administration can be local or systemic. For example, if the chosen route is intravascular, the agent (such as antibody) is administered by introducing the composition into a blood vessel of the subject.

Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Antibody and Antigen Binding Fragment: An immunoglobulin, antigen-binding fragment, or derivative thereof, that specifically binds and recognizes an analyte (antigen) such as a coronavirus spike protein, such as a spike protein from SARS-CoV-2. The term “antibody” is used herein in the broadest sense and encompasses various antibody structures, including but not limited to monoclonal antibodies, polyclonal antibodies, multispecific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Antigen binding fragments include a V_{H} and a V_{L}, and specifically bind the cognate antigen. Examples of antigen binding fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some embodiments, a disclosed antibody includes a heterologous constant domain. For example, the antibody includes a constant domain that is different from a native constant domain, such as a constant domain including one or more modifications (such as the “LS” mutation) to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.) A “humanized” antibody or antigen binding fragment includes a human framework region and one or more CDRs from a non-human (such as a mouse, rat, or synthetic) antibody or antigen binding fragment.

The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008).

Antibody or antigen binding fragment that neutralizes a coronavirus: An antibody or antigen binding fragment that specifically binds to a coronavirus antigen (such as the spike protein) in such a way as to inhibit a biological function associated with the coronavirus that inhibits infection. The antibody can neutralize the activity of one or more coronaviruses. For example, an antibody or antigen binding fragment that neutralizes the coronavirus, such as SARS-CoV-2 may interfere with the virus by binding it directly and limiting entry into cells. Alternately, an antibody may interfere with one or more post-attachment interactions of the pathogen with a receptor, for example, by interfering with viral entry using the receptor. In some examples, an antibody that is specific for a coronavirus spike protein neutralizes the infectious titer of the coronavirus.

In some embodiments, an antibody or antigen binding fragment that specifically binds to a coronavirus, and neutralizes the coronavirus, inhibits infection of cells, for example, by at least 50% compared to a control antibody or antigen binding fragment.

A “broadly neutralizing antibody” is an antibody that binds to and inhibits the function of related antigens, such as antigens that share at least 85%, 90%, 95%, 96%, 97%, 98% or 99% identity antigenic surface of antigen. With regard to an antigen from a pathogen, such as a virus, the antibody can bind to and inhibit the function of an antigen from more than one class and/or subclass of the pathogen. For example, with regard to a coronavirus, the antibody can bind to and inhibit the function of an antigen, such as the spike protein from coronaviruses.

Biological sample: A sample obtained from a subject. Biological samples include all clinical samples useful for detection of disease or infection in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having a coronavirus infection, such as, but not limited to, a SARS-CoV-2 infection.

Bispecific antibody: A recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain.

Conditions sufficient to form an immune complex: Conditions which allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Greenfield (Ed.), Antibodies: A Laboratory Manual, 2^{nd} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography.

Conjugate: A complex of two molecules linked together, for example, linked together by a covalent bond. In one embodiment, an antibody is linked to an effector molecule; for example, an antibody that specifically binds to one or more coronaviruses, such as SARS-CoV-2, covalently linked to an effector molecule, such as a detectable label. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, a coronavirus-specific antibody can include up to 1, 2, 3, 4, 5, 6, 7, 8, 9, or up to 10 conservative substitutions compared to a reference antibody sequence and retain specific binding activity for spike protein binding, and/or neutralization activity. The term conservative variation also includes the use of a substituted amino acid in place of an unsubstituted parent amino acid.

Individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the antibody, such as the ability to specifically bind to a coronavirus spike protein. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some embodiments, the control is a negative control, such as sample obtained from a healthy patient not infected a coronavirus. In other embodiments, the control is a positive control, such as a tissue sample obtained from a patient diagnosed with a coronavirus infection. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Coronavirus: A family of positive-sense, single-stranded RNA viruses that are known to cause severe respiratory illness. Viruses currently known to infect human from the coronavirus family are from the alphacoronavirus and betacoronavirus genera. Additionally, it is believed that the gammacoronavirus and deltacoronavirus genera may infect humans in the future.

Non-limiting examples of betacoronaviruses include SARS-CoV-2, Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV).

The viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene.

Degenerate variant: In the context of the present disclosure, a “degenerate variant” refers to a polynucleotide encoding a polypeptide (such as an antibody heavy or light chain) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

Effective amount: A quantity of a specific substance sufficient to achieve a desired effect in a subject to whom the substance is administered. For instance, this can be the amount necessary to inhibit a coronavirus infection, such as a SARS-CoV-2 infection, or to measurably alter outward symptoms of such an infection.

In one example, a desired response is to inhibit or reduce or prevent SARS-CoV-2 infection. The SARS-CoV-2 infection does not need to be completely eliminated or reduced or prevented for the method to be effective. For example, administration of an effective amount of the immunogen can induce an immune response that decreases the SARS-CoV-2 infection (for example, as measured by infection of cells, or by number or percentage of subjects infected by the SARS-CoV-2) by a desired amount, for example by at least 50%, at least 60%, at least 70%, at least 80%, at least 90%, at least 95%, at least 98%, or even at least 100% (elimination or prevention of detectable SARS-CoV-2 infection), as compared to a suitable control. Additional coronavirus infections can also be inhibited as a results of using the presently disclosed antibodies.

In some embodiments, administration of an effective amount of a disclosed antibody or antigen binding fragment that binds to a coronavirus spike protein can reduce or inhibit infection (for example, as measured by infection of cells, or by number or percentage of subjects infected by the coronavirus or by an increase in the survival time of infected subjects, or reduction in symptoms associated with the infection) by a desired amount, for example by at least 10%, at least 20%, at least 50%, at least 60%, at least 70%, at least 80%, at least 90%, at least 95%, at least 98%, or even at least 100% (elimination or prevention of detectable infection), as compared to a suitable control.

The effective amount of an antibody or antigen binding fragment that specifically binds the coronavirus spike protein that is administered to a subject to inhibit infection will vary depending upon a number of factors associated with that subject, for example the overall health and/or weight of the subject. An effective amount can be determined by varying the dosage and measuring the resulting response, such as, for example, a reduction in pathogen titer. Effective amounts also can be determined through various in vitro, in vivo or in situ immunoassays.

An effective amount encompasses a fractional dose that contributes in combination with previous or subsequent administrations to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the agent can be packaged in an amount, or in multiples of the effective amount, for example, in a vial (e.g., with a pierceable lid) or syringe having sterile components.

Effector molecule: A molecule intended to have or produce a desired effect; for example, a desired effect on a cell to which the effector molecule is targeted, or a detectable marker. Effector molecules can include, for example, polypeptides and small molecules. Some effector molecules may have or produce more than one desired effect.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on a coronavirus spike protein.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Fc region: The constant region of an antibody excluding the first heavy chain constant domain. Fc region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cγ2 and Cγ3 and optionally the lower part of the hinge between Cγ1 and Cγ2. Although the boundaries of the Fc region may vary, the human IgG heavy chain Fc region is usually defined to include residues following C226 or P230 to the Fc carboxyl-terminus, wherein the numbering is according to EU numbering. For IgA, the Fc region includes immunoglobulin domains Cα2 and Cα3 and optionally the lower part of the hinge between Cα1 and Cα2.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as an scFv, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Host cell: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

IgA: A polypeptide belonging to the class of antibodies that are substantially encoded by a recognized immunoglobulin alpha gene. In humans, this class or isotype comprises IgA₁ and IgA₂. IgA antibodies can exist as monomers, polymers (referred to as pIgA) of predominantly dimeric form, and secretory IgA. The constant chain of wild-type IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

IgG: A polypeptide belonging to the class or isotype of antibodies that are substantially encoded by a recognized immunoglobulin gamma gene. In humans, this class comprises IgG₁, IgG₂, IgG3, and IgG4.

Immune complex: The binding of antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Inhibiting or treating a disease: Inhibiting the full development of a disease or condition, for example, in a subject who is at risk for a disease such as a coronavirus infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

The term “reduces” is a relative term, such that an agent reduces a disease or condition if the disease or condition is quantitatively diminished following administration of the agent, or if it is diminished following administration of the agent, as compared to a reference agent. Similarly, the term “prevents” does not necessarily mean that an agent completely eliminates the disease or condition, so long as at least one characteristic of the disease or condition is eliminated. Thus, a composition that reduces or prevents an infection, can, but does not necessarily completely, eliminate such an infection, so long as the infection is measurably diminished, for example, by at least about 50%, such as by at least about 70%, or about 80%, or even by about 90% the infection in the absence of the agent, or in comparison to a reference agent.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Kabat position: A position of a residue in an amino acid sequence that follows the numbering convention delineated by Kabat et al. (Sequences of Proteins of Immunological Interest, 5^{th} Edition, Department of Health and Human Services, Public Health Service, National Institutes of Health, Bethesda, NIH Publication No. 91-3242, 1991).

Linker: A bi-functional molecule that can be used to link two molecules into one contiguous molecule, for example, to link a detectable marker to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some embodiments, the polypeptide is a disclosed antibody or a fragment thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one embodiment, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

SARS-CoV-2: Also known as Wuhan coronavirus or 2019 novel coronavirus, SARS-CoV-2 is a positive-sense, single stranded RNA virus of the genus betacoronavirus that has emerged as a highly fatal cause of severe acute respiratory infection. The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (N)-3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-CoV-2 infection, including but not limited to, assessment of patient symptoms and background and genetic tests such as reverse transcription-polymerase chain reaction (rRT-PCR). The test can be done on patient samples such as respiratory or blood samples.

Spike (S) protein (Coronavirus): A class I fusion glycoprotein initially synthesized as a precursor protein of approximately 1256 amino acids in size for SARS-CoV, and 1273 for SARS-CoV-2. Individual precursor S polypeptides form a homotrimer and undergo glycosylation within the Golgi apparatus as well as processing to remove the signal peptide, and cleavage by a cellular protease between approximately position 679/680 for SARS-CoV, and 685/686 for SARS-CoV-2, to generate separate S1 and S2 polypeptide chains, which remain associated as S1/S2 protomers within the homotrimer and is therefore a trimer of heterodimers. The S1 subunit is distal to the virus membrane and contains the receptor-binding domain (RBD) that is believed to mediate virus attachment to its host receptor. The S2 subunit contains the fusion protein machinery, such as the fusion peptide, two heptad-repeat sequences (HR1 and HR2) and a central helix typical of fusion glycoproteins, a transmembrane domain, and the cytosolic tail domain.

An exemplary sequence of SARS-CoV S protein is provided in GENBANK® GI: 30795145, as available on Feb. 1, 2022. An exemplary sequence of the HKU1-CoV S protein is provided in GENBANK® GI: 123867264, as available on Feb. 1, 2022. An exemplary sequence of OC43-CoV S protein is provided in GENBANK® GI: 744516696, as available on Feb. 1, 2022. An exemplary sequence of NL63-CoV S protein is provided in GENBANK® GI: 71153773, as available on Feb. 1, 2022. An exemplary sequence of 229E-CoV S protein is provided in GENBANK® GI: 1060650120, as available on Feb. 1, 2022. The S protein includes both the S1 and S2 domains.

The numbering used in the disclosed SARS-CoV-2 S proteins and fragments thereof is relative to the S protein of SARS-CoV-2, the sequence of which was deposited as NCBI Ref. No. YP_009724390.1, as available on Feb. 1, 2022, which is incorporated by reference herein in its entirety.

Sequence identity: The identity between two or more nucleic acid sequences, or two or more amino acid sequences, is expressed in terms of the percentage identity between the sequences. Sequence identity can be measured in terms of percentage identity; the higher the percentage, the more identical the sequences. Homologs and variants of a V_{L} or a V_{H} of an antibody that specifically binds a target antigen are typically characterized by possession of at least about 75% sequence identity, for example at least about 80%, 85%, 90%, 91%, 92%, 93%, 94%, 95%, 96%, 97%, 98% or 99% sequence identity counted over the full-length alignment with the amino acid sequence of interest.

Any suitable method may be used to align sequences for comparison. Non-limiting examples of programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics, 5(2):151-3, 1989; Corpet, Nucleic Acids Res. 16(22):10881-10890, 1988; Huang et al. Bioinformatics, 8(2):155-165, 1992; and Pearson, Methods Mol. Biol. 24:307-331, 1994., Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example a coronavirus spike protein and does not bind in a significant amount to other proteins present in the sample or subject. With regard to a spike protein, the epitope may be present on the spike protein of more than one type of coronavirus, such that the antibody binds to the spike protein on more than one types of virus, but does not bind to other proteins, such as proteins from other viruses or other proteins (non-spike) of coronavirus. Specific binding can be determined by standard methods. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹¹ Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

An antibody that specifically binds to an epitope on a coronavirus spike protein, such as the S domain, RBD domain, or NTD domain, can bind molecules/agents including this domain, including viruses, substrate to which the spike protein is attached, or the protein in a biological specimen. It is, of course, recognized that a certain degree of non-specific interaction may occur between an antibody and a non-target. Typically, specific binding results in a much stronger association between the antibody and a spike protein than between the antibody other different coronavirus proteins (such as the E, M or N protein) or from non-coronavirus proteins. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein.

Subject: Living multi-cellular vertebrate organisms, a category that includes human and non-human mammals, such as non-human primates, pigs, camels, bats, sheep, cows, dogs, cats, rodents, and the like. In an example, a subject is a human. In a particular example, the subject is a human. In an additional example, a subject is selected that is in need of inhibiting a SARS-CoV-2 infection. For example, the subject is either uninfected and at risk of the SARS-CoV-2 infection or is infected and in need of treatment.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. In some embodiments, a viral vector comprises a nucleic acid molecule encoding a disclosed antibody or antigen binding fragment that specifically binds to a coronavirus spike protein and neutralizes the coronavirus. In some embodiments, the viral vector can be an adeno-associated virus (AAV) vector.

Under conditions sufficient for: A phrase that is used to describe any environment that permits a desired activity.

### II. Description of Several Embodiments

Isolated monoclonal antibodies and antigen binding fragments that specifically bind a coronavirus spike protein are provided. The monoclonal antibodies and antigen binding fragments specifically bind to a coronavirus spike protein and neutralize SARS-CoV-2 and at least one additional betacoronavirus or alphacoronavirus. The antibodies and antigen binding fragments can be fully human. The antibodies and antigen binding fragments can neutralize a coronavirus, such as, but not limited to, SARS-CoV-2. In some embodiments the disclosed antibodies can inhibit a coronavirus infection in vivo, and can be administered prior to, or after, an infection with a coronavirus, such as, but not limited to, SARS-CoV-2. Bispecific antibodies including the variable domains of these antibodies are also provided. In addition, disclosed herein are compositions comprising the antibodies and antigen binding fragments and a pharmaceutically acceptable carrier. Nucleic acids encoding the antibodies, antigen binding fragments, variable domains, and expression vectors (such as adeno-associated virus (AAV) viral vectors) comprising these nucleic acids are also provided. The antibodies, antigen binding fragments, nucleic acid molecules, host cells, and compositions can be used for research, diagnostic, treatment and prophylactic purposes. For example, the disclosed antibodies and antigen binding fragments can be used to diagnose a subject with a coronavirus infection or can be administered to inhibit a coronavirus infection in a subject.

A. Monoclonal Antibodies that Specifically Bind a Coronavirus Spike Protein and Antigen Binding Fragments Thereof

The discussion of monoclonal antibodies below refers to isolated monoclonal antibodies that include heavy and/or light chain variable domains (or antigen binding fragments thereof) comprising a CDR1, CDR2, and/or CDR3 with reference to the IMGT numbering scheme (unless the context indicates otherwise). Various CDR numbering schemes (such as the Kabat, Chothia or IMGT numbering schemes) can be used to determine CDR positions. The amino acid sequence and the CDRs of the heavy and light chain of the disclosed monoclonal antibody according to the IMGT numbering scheme are provided in the listing of sequences, but these are exemplary only.

In some embodiments, a monoclonal antibody is provided that comprises the heavy and light chain CDRs of any one of the antibodies described herein. In some embodiment, a monoclonal antibody is provided that comprises the heavy and light chain variable regions of any one of the antibodies described herein.

In some embodiments, the antibody binds the N-terminal domain of the coronavirus spike protein. In other embodiments, the antibody binds an S domain of the coronavirus spike protein. In further embodiments, the antibody binds a stem helix in the S2 domain of the spike protein, such as LQPELDSFKEELDKYFKNHTS (SEQ ID NO: 489), such as LDSFKEELDKYF (SEQ ID NO: 490).

In some embodiments, the antibody or antigen binding fragment specifically binds a spike protein from at least three betacoronaviruses selected from the group consisting of SARS-CoV-2, SARS-CoV, MERS-CoV, HKU1, and OC43. In other embodiments, the antibody or antigen binding fragment neutralizes a betacoronavirus and an alphacoronavirus. In more embodiments, the antibody or antigen binding fragment specifically binds a spike protein from SARS-CoV-2, SARS-CoV, MERS-CoV, HKU1, OC43, NL63 and 229E.

Binding data is shown in the accompanying figures for the antibodies described below.

a. Monoclonal Antibody COV44-79

In some embodiments, the antibody or antigen binding fragment is based on or derived from the COV44-79 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV44-79 antibody, and specifically binds to a coronavirus spike protein, and neutralizes a coronavirus. The coronavirus can be SARS-CoV-2. In some embodiments, the antibody or antigen binding fragment neutralizes a betacoronavirus and an alphacoronavirus. In some embodiments, the antibody or antigen binding fragment specifically binds a spike protein from SARS-CoV-2, SARS-CoV, MERS-CoV, HKU1, OC43, NL63 and 229E.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 17, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 21, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 17 and 21, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 18, 19, and 20, respectively, and/or a V_{L} comprising a LCDR1, a LCDR2, and a LCDR3 as set forth as SEQ ID NOs: 22, 23, and 24, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 18, 19, and 20, respectively, a V_{L} comprising a LCDR1, a LCDR2, and a LCDR3 as set forth as SEQ ID NOs: 22, 23, and 24, respectively, wherein the V_{H} comprises an amino acid sequence at least 90% identical to SEQ ID NO: 17, such as 95%, 96%, 97%, 98% or 99% identical to SEQ ID NO: 17, and wherein the V_{L} comprises an amino acid sequence at least 90% identical to SEQ ID NO: 21, such as 95%, 96%, 97%, 98% or 99% identical to SEQ ID NO: 21, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 17, and specifically binds to a coronavirus spike protein, and neutralizes a coronavirus. In more embodiments, the antibody or antigen binding fragment comprises a V_{L} comprising the amino acid sequence set forth as SEQ ID NO: 21, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 17 and 21, respectively, and specifically binds to a coronavirus spike protein, and neutralizes a coronavirus. The coronavirus can be SARS-CoV-2.

In some embodiments, the disclosed antibodies inhibit viral entry and/or replication.

b. Monoclonal Antibody COV44-62

In some embodiments, the antibody or antigen binding fragment is based on or derived from the COV44-62 antibody, and specifically binds to a coronavirus spike protein, and neutralizes a coronavirus. In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV44-62 antibody, and specifically binds to a coronavirus spike protein, and neutralizes a coronavirus. The coronavirus can be SARS-CoV-2. In some embodiments, the antibody or antigen binding fragment neutralizes a betacoronavirus and an alphacoronavirus. In some embodiments, the antibody or antigen binding fragment specifically binds a spike protein from SARS-CoV-2, SARS-CoV, MERS-CoV, HKU1, OC43, NL63 and 229E.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 9, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 13, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 9 and 13, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 10, 11, and 12, respectively, and/or a V_{L} comprising a LCDR1, a LCDR2, and a LCDR3 as set forth as SEQ ID NOs: 14, 15 and 16, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 10, 11, and 12, respectively, a V_{L} comprising a LCDR1, a LCDR2, and a LCDR3 as set forth as SEQ ID NOs: 14, 15 and 16, respectively, wherein the V_{H} comprises an amino acid sequence at least 90% identical to SEQ ID NO: 9, such as 95%, 96%, 97%, 98% or 99% identical to SEQ ID NO: 9, and wherein the V_{L} comprises an amino acid sequence at least 90% identical to SEQ ID NO: 13, such as 95%, 96%, 97%, 98% or 99% identical to SEQ ID NO: 13, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 9, and specifically binds to a coronavirus spike protein, and neutralizes a coronavirus. In more embodiments, the antibody or antigen binding fragment comprises a V_{L} comprising the amino acid sequence set forth as SEQ ID NO: 13, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 9 and 13, respectively, and specifically binds to a coronavirus spike protein, and neutralizes a coronavirus. The coronavirus can be SARS-CoV-2.

In some embodiments, the disclosed antibodies inhibit viral entry and/or replication.

c. Monoclonal Antibody COV89-22

In some embodiments, the antibody or antigen binding fragment is based on or derived from the COV89-22 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV89-22 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment neutralizes a betacoronavirus and an alphacoronavirus. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 5, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 5, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, 7, and 8, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, 7, and 8 respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 5, such as 95%, 96%, 97%, 98% or 99% identical to SEQ ID NO: 5, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 1, and specifically binds to a coronavirus spike protein, and neutralizes a coronavirus. In more embodiments, the antibody or antigen binding fragment comprises a V_{L} comprising the amino acid sequence set forth as SEQ ID NO: 5, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 1 and 5, respectively, and specifically binds to a coronavirus spike protein, and neutralizes a coronavirus. The coronavirus can be SARS-CoV-2.

In some embodiments, the disclosed antibodies inhibit viral entry and/or replication.

d. Monoclonal Antibody COV30-14

In some embodiments, the antibody or antigen binding fragment is based on or derived from the COV30-14 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV30-14 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment neutralizes a betacoronavirus and an alphacoronavirus. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 25, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 29, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 25 and 29, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 26, 27, and 28, respectively, and/or a V_{L} comprising a LCDR1, a LCDR2, and a LCDR3 as set forth as SEQ ID NOs: 30, 31, and 32, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 26, 27, and 28, respectively, a V_{L} comprising a LCDR1, a LCDR2, and a LCDR3 as set forth as SEQ ID NOs: 30, 31, and 32, respectively, wherein the V_{H} comprises an amino acid sequence at least 90% identical to SEQ ID NO: 25, such as 95%, 96%, 97%, 98% or 99% identical to SEQ ID NO: 25, and wherein the V_{L} comprises an amino acid sequence at least 90% identical to SEQ ID NO: 29, such as 95%, 96%, 97%, 98% or 99% identical to SEQ ID NO: 29, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 25, and specifically binds to a coronavirus spike protein, and neutralizes a coronavirus. In more embodiments, the antibody or antigen binding fragment comprises a V_{L} comprising the amino acid sequence set forth as SEQ ID NO: 29, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 25 and 29, respectively, and specifically binds to a coronavirus spike protein, and neutralizes a coronavirus. The coronavirus can be SARS-CoV-2.

In some embodiments, the disclosed antibodies inhibit viral entry and/or replication.

e. Monoclonal Antibody COV72-37

In some embodiments, the antibody or antigen binding fragment is based on or derived from the COV72-37 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV72-37 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment neutralizes a betacoronavirus and an alphacoronavirus. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 33, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 37, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 33 and 37, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 34, 35, and 36, respectively, and/or a V_{L} comprising a LCDR1, a LCDR2, and a LCDR3 as set forth as SEQ ID NOs: 38, 39, and 40, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 34, 35, and 36, respectively, a V_{L} comprising a LCDR1, a LCDR2, and a LCDR3 as set forth as SEQ ID NOs: 38, 39, and 40, respectively, wherein the V_{H} comprises an amino acid sequence at least 90% identical to SEQ ID NO: 33, such as 95%, 96%, 97%, 98% or 99% identical to SEQ ID NO: 33, and wherein the V_{L} comprises an amino acid sequence at least 90% identical to SEQ ID NO: 37, such as 95%, 96%, 97%, 98% or 99% identical to SEQ ID NO: 37, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 33, and specifically binds to a coronavirus spike protein, and neutralizes a coronavirus. In more embodiments, the antibody or antigen binding fragment comprises a V_{L} comprising the amino acid sequence set forth as SEQ ID NO: 37, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 33 and 37, respectively, and specifically binds to a coronavirus spike protein, and neutralizes a coronavirus. The coronavirus can be SARS-CoV-2.

In some embodiments, the disclosed antibodies inhibit viral entry and/or replication.

f. Monoclonal Antibody COV91-27

In some embodiments, the antibody or antigen binding fragment is based on or derived from the COV91-27 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV91-27 antibody, and specifically binds to a coronavirus spike protein, and neutralizes a coronavirus. The coronavirus can be SARS-CoV-2. In some embodiments, the antibody or antigen binding fragment specifically binds a spike protein from SARS-CoV-2, SARS-CoV, MERS-CoV, HKU1, OC43, NL63 and 229E. In some embodiments, the antibody or antigen binding fragment neutralizes a betacoronavirus and an alphacoronavirus.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 49, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 53, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 49 and 53, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 50, 51, and 52, respectively, and/or a V_{L} comprising a LCDR1, a LCDR2, and a LCDR3 as set forth as SEQ ID NOs: 54, 55, and 56, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 50, 51, and 52, respectively, a V_{L} comprising a LCDR1, a LCDR2, and a LCDR3 as set forth as SEQ ID NOs: 54, 55, and 56, respectively, wherein the V_{H} comprises an amino acid sequence at least 90% identical to SEQ ID NO: 49, such as 95%, 96%, 97%, 98% or 99% identical to SEQ ID NO: 49, and wherein the V_{L} comprises an amino acid sequence at least 90% identical to SEQ ID NO: 53, such as 95%, 96%, 97%, 98% or 99% identical to SEQ ID NO: 53, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 49, and specifically binds to a coronavirus spike protein, and neutralizes a coronavirus. In more embodiments, the antibody or antigen binding fragment comprises a V_{L} comprising the amino acid sequence set forth as SEQ ID NO: 53, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 49 and 53, respectively, and specifically binds to a coronavirus spike protein, and neutralizes a coronavirus. The coronavirus can be SARS-CoV-2.

In some embodiments, the disclosed antibodies inhibit viral entry and/or replication.

g. Monoclonal Antibody COV93-03

In some embodiments, the antibody or antigen binding fragment is based on or derived from the COV93-03 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV93-03 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment neutralizes a betacoronavirus and an alphacoronavirus. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 41, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 45, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 41 and 45, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 42, 43, and 44, respectively, and/or a V_{L} comprising a LCDR1, a LCDR2, and a LCDR3 as set forth as SEQ ID NOs: 46, 47, and 48, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 42, 43, and 44, respectively, a V_{L} comprising a LCDR1, a LCDR2, and a LCDR3 as set forth as SEQ ID NOs: 46, 47, and 48, respectively, wherein the V_{H} comprises an amino acid sequence at least 90% identical to SEQ ID NO: 41, such as 95%, 96%, 97%, 98% or 99% identical to SEQ ID NO: 41, and wherein the V_{L} comprises an amino acid sequence at least 90% identical to SEQ ID NO: 45, such as 95%, 96%, 97%, 98% or 99% identical to SEQ ID NO: 45, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 41, and specifically binds to a coronavirus spike protein, and neutralizes a coronavirus. In more embodiments, the antibody or antigen binding fragment comprises a V_{L} comprising the amino acid sequence set forth as SEQ ID NO: 45, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 41 and 45, respectively, and specifically binds to a coronavirus spike protein, and neutralizes a coronavirus. The coronavirus can be SARS-CoV-2.

In some embodiments, the disclosed antibodies inhibit viral entry and/or replication.

h. Monoclonal Antibody COV49-51

In some embodiments, the antibody or antigen binding fragment is based on or derived from the COV49-51 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-51 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment neutralizes a betacoronavirus and an alphacoronavirus. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 57, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 61, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 57 and 61, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 58, 59, and 60, respectively, and/or a V_{L} comprising a LCDR1, a LCDR2, and a LCDR3 as set forth as SEQ ID NOs: 62, 63, and 64, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 58, 59, and 60, respectively, a V_{L} comprising a LCDR1, a LCDR2, and a LCDR3 as set forth as SEQ ID NOs: 62, 63, and 64, respectively, wherein the V_{H} comprises an amino acid sequence at least 90% identical to SEQ ID NO: 57, such as 95%, 96%, 97%, 98% or 99% identical to SEQ ID NO: 57, and wherein the V_{L} comprises an amino acid sequence at least 90% identical to SEQ ID NO: 61, such as 95%, 96%, 97%, 98% or 99% identical to SEQ ID NO: 61, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 57, and specifically binds to a coronavirus spike protein, and neutralizes a coronavirus. In more embodiments, the antibody or antigen binding fragment comprises a V_{L} comprising the amino acid sequence set forth as SEQ ID NO: 61, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 57 and 61, respectively, and specifically binds to a coronavirus spike protein, and neutralizes a coronavirus. The coronavirus can be SARS-CoV-2.

In some embodiments, the disclosed antibodies inhibit viral entry and/or replication.

i. Monoclonal Antibody COV44-74

In some embodiments, the antibody or antigen binding fragment is based on or derived from the COV44-74 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV44-74 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment neutralizes a betacoronavirus and an alphacoronavirus. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 65, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 69, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 65 and 69, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 66, 67, and 68, respectively, and/or a V_{L} comprising a LCDR1, a LCDR2, and a LCDR3 as set forth as SEQ ID NOs: 70, 71, and 72, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 66, 67, and 68, respectively, a V_{L} comprising a LCDR1, a LCDR2, and a LCDR3 as set forth as SEQ ID NOs: 70, 71, and 72, respectively, wherein the V_{H} comprises an amino acid sequence at least 90% identical to SEQ ID NO: 65, such as 95%, 96%, 97%, 98% or 99% identical to SEQ ID NO: 65, and wherein the V_{L} comprises an amino acid sequence at least 90% identical to SEQ ID NO: 69, such as 95%, 96%, 97%, 98% or 99% identical to SEQ ID NO: 69, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 65, and specifically binds to a coronavirus spike protein, and neutralizes a coronavirus. In more embodiments, the antibody or antigen binding fragment comprises a V_{L} comprising the amino acid sequence set forth as SEQ ID NO: 69, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 65 and 69, respectively, and specifically binds to a coronavirus spike protein, and neutralizes a coronavirus. The coronavirus can be SARS-CoV-2.

In some embodiments, the disclosed antibodies inhibit viral entry and/or replication.

j. Monoclonal Antibody COV44-56

In some embodiments, the antibody or antigen binding fragment is based on or derived from the COV44-56 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV44-56 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment neutralizes a betacoronavirus and an alphacoronavirus. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 73, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 77, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 73 and 77, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 74, 75, and 76, respectively, and/or a V_{L} comprising a LCDR1, a LCDR2, and a LCDR3 as set forth as SEQ ID NOs: 78, 79, and 80, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 74, 75, and 76, respectively, a V_{L} comprising a LCDR1, a LCDR2, and a LCDR3 as set forth as SEQ ID NOs: 78, 79, and 80, respectively, wherein the V_{H} comprises an amino acid sequence at least 90% identical to SEQ ID NO: 73, such as 95%, 96%, 97%, 98% or 99% identical to SEQ ID NO: 73, and wherein the V_{L} comprises an amino acid sequence at least 90% identical to SEQ ID NO: 77, such as 95%, 96%, 97%, 98% or 99% identical to SEQ ID NO: 77, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 73 and specifically binds to a coronavirus spike protein, and neutralizes a coronavirus. In more embodiments, the antibody or antigen binding fragment comprises a V_{L} comprising the amino acid sequence set forth as SEQ ID NO: 77, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 73 and 77, respectively, and specifically binds to a coronavirus spike protein, and neutralizes a coronavirus. The coronavirus can be SARS-CoV-2.

In some embodiments, the disclosed antibodies inhibit viral entry and/or replication.

k. Monoclonal Antibody COV44-26

In some embodiments, the antibody or antigen binding fragment is based on or derived from the COV44-26 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV44-26 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment neutralizes a betacoronavirus and an alphacoronavirus. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 81, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 85, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 81 and 85, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 82, 83, and 84, respectively, and/or a V_{L} comprising a LCDR1, a LCDR2, and a LCDR3 as set forth as SEQ ID NOs: 86, 87, and 88, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 82, 83, and 84, respectively, a V_{L} comprising a LCDR1, a LCDR2, and a LCDR3 as set forth as SEQ ID NOs: 86, 87, and 88, respectively, wherein the V_{H} comprises an amino acid sequence at least 90% identical to SEQ ID NO: 81, such as 95%, 96%, 97%, 98% or 99% identical to SEQ ID NO: 81, and wherein the V_{L} comprises an amino acid sequence at least 90% identical to SEQ ID NO: 85, such as 95%, 96%, 97%, 98% or 99% identical to SEQ ID NO: 85, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 81, and specifically binds to a coronavirus spike protein, and neutralizes a coronavirus. In more embodiments, the antibody or antigen binding fragment comprises a V_{L} comprising the amino acid sequence set forth as SEQ ID NO: 85, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 81 and 85, respectively, and specifically binds to a coronavirus spike protein, and neutralizes a coronavirus. The coronavirus can be SARS-CoV-2.

In some embodiments, the disclosed antibodies inhibit viral entry and/or replication.

l. Monoclonal Antibody COV44-54

In some embodiments, the antibody or antigen binding fragment is based on or derived from the COV44-54 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV44-54 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment neutralizes a betacoronavirus and an alphacoronavirus. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 89, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 93, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 89 and 93, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 90, 91, and 92, respectively, and/or a V_{L} comprising a LCDR1, a LCDR2, and a LCDR3 as set forth as SEQ ID NOs: 94, 95, and 96, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 90, 91, and 92, respectively, a V_{L} comprising a LCDR1, a LCDR2, and a LCDR3 as set forth as SEQ ID NOs: 94, 95, and 96, respectively, wherein the V_{H} comprises an amino acid sequence at least 90% identical to SEQ ID NO: 89, such as 95%, 96%, 97%, 98% or 99% identical to SEQ ID NO: 89, and wherein the V_{L} comprises an amino acid sequence at least 90% identical to SEQ ID NO: 93, such as 95%, 96%, 97%, 98% or 99% identical to SEQ ID NO: 93, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 89, and specifically binds to a coronavirus spike protein, and neutralizes a coronavirus. In more embodiments, the antibody or antigen binding fragment comprises a V_{L} comprising the amino acid sequence set forth as SEQ ID NO: 93, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 89 and 93, respectively, and specifically binds to a coronavirus spike protein, and neutralizes a coronavirus. The coronavirus can be SARS-CoV-2.

In some embodiments, the disclosed antibodies inhibit viral entry and/or replication.

m. Monoclonal Antibody COV23-01

In some embodiments, the antibody or antigen binding fragment is based on or derived from the COV23-01 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV23-01 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 97, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 101, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 97 and 101, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 98, 99, and 100, respectively, and/or a V_{L} comprising a LCDR1, a LCDR2, and a LCDR3 as set forth as SEQ ID NOs: 102, 103, and 104, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 98, 99, and 100, respectively, a V_{L} comprising a LCDR1, a LCDR2, and a LCDR3 as set forth as SEQ ID NOs: 102, 103, and 104, respectively, wherein the V_{H} comprises an amino acid sequence at least 90% identical to SEQ ID NO: 97, such as 95%, 96%, 97%, 98% or 99% identical to SEQ ID NO: 97, and wherein the V_{L} comprises an amino acid sequence at least 90% identical to SEQ ID NO: 101, such as 95%, 96%, 97%, 98% or 99% identical to SEQ ID NO: 101, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 97, and specifically binds to a coronavirus spike protein, and neutralizes a coronavirus. In more embodiments, the antibody or antigen binding fragment comprises a V_{L} comprising the amino acid sequence set forth as SEQ ID NO: 101, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 97 and 101, respectively, and specifically binds to a coronavirus spike protein, and neutralizes a coronavirus. The coronavirus can be SARS-CoV-2.

In some embodiments, the disclosed antibodies inhibit viral entry and/or replication.

n. Monoclonal Antibody COV49-03

In some embodiments, the antibody or antigen binding fragment is based on or derived from the COV49-03 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-03 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 105, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 109, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 105 and 109, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 106, 107, and 108, respectively, and/or a V_{L} comprising a LCDR1, a LCDR2, and a LCDR3 as set forth as SEQ ID NOs: 110, 111, and 112, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 106, 107, and 108, respectively, a V_{L} comprising a LCDR1, a LCDR2, and a LCDR3 as set forth as SEQ ID NOs: 110, 111, and 112, respectively, wherein the V_{H} comprises an amino acid sequence at least 90% identical to SEQ ID NO: 105, such as 95%, 96%, 97%, 98% or 99% identical to SEQ ID NO: 105, and wherein the V_{L} comprises an amino acid sequence at least 90% identical to SEQ ID NO: 109, such as 95%, 96%, 97%, 98% or 99% identical to SEQ ID NO: 109, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 105, and specifically binds to a coronavirus spike protein, and neutralizes a coronavirus. In more embodiments, the antibody or antigen binding fragment comprises a V_{L} comprising the amino acid sequence set forth as SEQ ID NO: 109, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 105 and 109, respectively, and specifically binds to a coronavirus spike protein, and neutralizes a coronavirus. The coronavirus can be SARS-CoV-2.

In some embodiments, the disclosed antibodies inhibit viral entry and/or replication.

o. Monoclonal Antibody COV49-04

In some embodiments, the antibody or antigen binding fragment is based on or derived from the COV49-04 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-04 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 113, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 117, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 113 and 117, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 114, 115, and 116, respectively, and/or a V_{L} comprising a LCDR1, a LCDR2, and a LCDR3 as set forth as SEQ ID NOs: 118, 119, and 120, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 114, 115, and 116, respectively, a V_{L} comprising a LCDR1, a LCDR2, and a LCDR3 as set forth as SEQ ID NOs: 118, 119, and 120, respectively, wherein the V_{H} comprises an amino acid sequence at least 90% identical to SEQ ID NO: 113, such as 95%, 96%, 97%, 98% or 99% identical to SEQ ID NO: 113, and wherein the V_{L} comprises an amino acid sequence at least 90% identical to SEQ ID NO: 117, such as 95%, 96%, 97%, 98% or 99% identical to SEQ ID NO: 117, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 113, and specifically binds to a coronavirus spike protein, and neutralizes a coronavirus. In more embodiments, the antibody or antigen binding fragment comprises a V_{L} comprising the amino acid sequence set forth as SEQ ID NO: 117, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 113 and 117, respectively, and specifically binds to a coronavirus spike protein, and neutralizes a coronavirus. The coronavirus can be SARS-CoV-2.

In some embodiments, the disclosed antibodies inhibit viral entry and/or replication.

p. Monoclonal Antibody COV49-05

In some embodiments, the antibody or antigen binding fragment is based on or derived from the COV49-05 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-05 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 121, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 125, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 121 and 125, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 122, 123, and 124, respectively, and/or a V_{L} comprising a LCDR1, a LCDR2, and a LCDR3 as set forth as SEQ ID NOs: 126, 127, and 128, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 122, 123, and 124, respectively, a V_{L} comprising a LCDR1, a LCDR2, and a LCDR3 as set forth as SEQ ID NOs: 126, 127, and 128, respectively, wherein the V_{H} comprises an amino acid sequence at least 90% identical to SEQ ID NO: 121, such as 95%, 96%, 97%, 98% or 99% identical to SEQ ID NO: 121, and wherein the V_{L} comprises an amino acid sequence at least 90% identical to SEQ ID NO: 125, such as 95%, 96%, 97%, 98% or 99% identical to SEQ ID NO: 125, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 121, and specifically binds to a coronavirus spike protein, and neutralizes a coronavirus. In more embodiments, the antibody or antigen binding fragment comprises a V_{L} comprising the amino acid sequence set forth as SEQ ID NO: 125, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 121 and 125, respectively, and specifically binds to a coronavirus spike protein, and neutralizes a coronavirus. The coronavirus can be SARS-CoV-2.

In some embodiments, the disclosed antibodies inhibit viral entry and/or replication.

q. Monoclonal Antibody COV49-06

In some embodiments, the antibody or antigen binding fragment is based on or derived from the COV49-06 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-06 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 129, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 133, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 129 and 133, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 130, 131, and 132, respectively, and/or a V_{L} comprising a LCDR1, a LCDR2, and a LCDR3 as set forth as SEQ ID NOs: 134, 135, and 136, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 130, 131, and 132, respectively, a V_{L} comprising a LCDR1, a LCDR2, and a LCDR3 as set forth as SEQ ID NOs: 134, 135, and 136, respectively, wherein the V_{H} comprises an amino acid sequence at least 90% identical to SEQ ID NO: 129, such as 95%, 96%, 97%, 98% or 99% identical to SEQ ID NO: 129, and wherein the V_{L} comprises an amino acid sequence at least 90% identical to SEQ ID NO: 133, such as 95%, 96%, 97%, 98% or 99% identical to SEQ ID NO: 133, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 129, and specifically binds to a coronavirus spike protein, and neutralizes a coronavirus. In more embodiments, the antibody or antigen binding fragment comprises a V_{L} comprising the amino acid sequence set forth as SEQ ID NO: 133, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 129 and 133, respectively, and specifically binds to a coronavirus spike protein, and neutralizes a coronavirus. The coronavirus can be SARS-CoV-2.

In some embodiments, the disclosed antibodies inhibit viral entry and/or replication.

r. Monoclonal Antibody COV49-07

In some embodiments, the antibody or antigen binding fragment is based on or derived from the COV49-07 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-07 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 137, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 141, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 137 and 141, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 138, 139, and 140, respectively, and/or a V_{L} comprising a LCDR1, a LCDR2, and a LCDR3 as set forth as SEQ ID NOs: 142, 143, and 144, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 138, 139, and 140, respectively, a V_{L} comprising a LCDR1, a LCDR2, and a LCDR3 as set forth as SEQ ID NOs: 142, 143, and 144, respectively, wherein the V_{H} comprises an amino acid sequence at least 90% identical to SEQ ID NO: 137, such as 95%, 96%, 97%, 98% or 99% identical to SEQ ID NO: 137, and wherein the V_{L} comprises an amino acid sequence at least 90% identical to SEQ ID NO: 141, such as 95%, 96%, 97%, 98% or 99% identical to SEQ ID NO: 141, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 137, and specifically binds to a coronavirus spike protein, and neutralizes a coronavirus. In more embodiments, the antibody or antigen binding fragment comprises a V_{L} comprising the amino acid sequence set forth as SEQ ID NO: 141, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 137 and 141, respectively, and specifically binds to a coronavirus spike protein, and neutralizes a coronavirus. The coronavirus can be SARS-CoV-2.

In some embodiments, the disclosed antibodies inhibit viral entry and/or replication.

s. Monoclonal Antibody COV49-18

In some embodiments, the antibody or antigen binding fragment is based on or derived from the COV49-18 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-18 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 145, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 149, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 145 and 149, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 146, 147, and 148, respectively, and/or a V_{L} comprising a LCDR1, a LCDR2, and a LCDR3 as set forth as SEQ ID NOs: 150, 151, and 152, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 146, 147, and 148, respectively, a V_{L} comprising a LCDR1, a LCDR2, and a LCDR3 as set forth as SEQ ID NOs: 150, 151, and 152, respectively, wherein the V_{H} comprises an amino acid sequence at least 90% identical to SEQ ID NO: 145, such as 95%, 96%, 97%, 98% or 99% identical to SEQ ID NO: 145, and wherein the V_{L} comprises an amino acid sequence at least 90% identical to SEQ ID NO: 149, such as 95%, 96%, 97%, 98% or 99% identical to SEQ ID NO: 149, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 145, and specifically binds to a coronavirus spike protein, and neutralizes a coronavirus. In more embodiments, the antibody or antigen binding fragment comprises a V_{L} comprising the amino acid sequence set forth as SEQ ID NO: 149, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 145 and 149, respectively, and specifically binds to a coronavirus spike protein, and neutralizes a coronavirus. The coronavirus can be SARS-CoV-2.

In some embodiments, the disclosed antibodies inhibit viral entry and/or replication.

t. Monoclonal Antibody COV49-23

In some embodiments, the antibody or antigen binding fragment is based on or derived from the COV49-23 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-23 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 153, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 157, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 153 and 157, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 154, 155, and 156, respectively, and/or a V_{L} comprising a LCDR1, a LCDR2, and a LCDR3 as set forth as SEQ ID NOs: 158, 159, and 160, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 154, 155, and 156, respectively, a V_{L} comprising a LCDR1, a LCDR2, and a LCDR3 as set forth as SEQ ID NOs: 158, 159, and 160, respectively, wherein the V_{H} comprises an amino acid sequence at least 90% identical to SEQ ID NO: 153, such as 95%, 96%, 97%, 98% or 99% identical to SEQ ID NO: 153, and wherein the V_{L} comprises an amino acid sequence at least 90% identical to SEQ ID NO: 157, such as 95%, 96%, 97%, 98% or 99% identical to SEQ ID NO: 157, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 153, and specifically binds to a coronavirus spike protein, and neutralizes a coronavirus. In more embodiments, the antibody or antigen binding fragment comprises a V_{L} comprising the amino acid sequence set forth as SEQ ID NO: 157, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 153 and 157, respectively, and specifically binds to a coronavirus spike protein, and neutralizes a coronavirus. The coronavirus can be SARS-CoV-2.

In some embodiments, the disclosed antibodies inhibit viral entry and/or replication.

u. Monoclonal Antibody COV49-28

In some embodiments, the antibody or antigen binding fragment is based on or derived from the COV49-28 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-28 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 161, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 165, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 161 and 165, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 162, 163, and 164, respectively, and/or a V_{L} comprising a LCDR1, a LCDR2, and a LCDR3 as set forth as SEQ ID NOs: 166, 167, and 168, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 162, 163, and 164, respectively, a V_{L} comprising a LCDR1, a LCDR2, and a LCDR3 as set forth as SEQ ID NOs: 166, 167, and 168, respectively, wherein the V_{H} comprises an amino acid sequence at least 90% identical to SEQ ID NO: 161, such as 95%, 96%, 97%, 98% or 99% identical to SEQ ID NO: 161, and wherein the V_{L} comprises an amino acid sequence at least 90% identical to SEQ ID NO: 165, such as 95%, 96%, 97%, 98% or 99% identical to SEQ ID NO: 165, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 161, and specifically binds to a coronavirus spike protein, and neutralizes a coronavirus. In more embodiments, the antibody or antigen binding fragment comprises a V_{L} comprising the amino acid sequence set forth as SEQ ID NO: 165, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 161 and 165, respectively, and specifically binds to a coronavirus spike protein, and neutralizes a coronavirus. The coronavirus can be SARS-CoV-2.

In some embodiments, the disclosed antibodies inhibit viral entry and/or replication.

v. Monoclonal Antibody COV49-30

In some embodiments, the antibody or antigen binding fragment is based on or derived from the COV49-30 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-30 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 169, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 173, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 169 and 173, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 170, 171, and 172, respectively, and/or a V_{L} comprising a LCDR1, a LCDR2, and a LCDR3 as set forth as SEQ ID NOs: 174, 175, and 176, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 170, 171, and 172, respectively, a V_{L} comprising a LCDR1, a LCDR2, and a LCDR3 as set forth as SEQ ID NOs: 174, 175, and 176, respectively, wherein the V_{H} comprises an amino acid sequence at least 90% identical to SEQ ID NO: 169, such as 95%, 96%, 97%, 98% or 99% identical to SEQ ID NO: 169, and wherein the V_{L} comprises an amino acid sequence at least 90% identical to SEQ ID NO: 173, such as 95%, 96%, 97%, 98% or 99% identical to SEQ ID NO: 173, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 169, and specifically binds to a coronavirus spike protein, and neutralizes a coronavirus. In more embodiments, the antibody or antigen binding fragment comprises a V_{L} comprising the amino acid sequence set forth as SEQ ID NO: 173, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 169 and 173, respectively, and specifically binds to a coronavirus spike protein, and neutralizes a coronavirus. The coronavirus can be SARS-CoV-2.

In some embodiments, the disclosed antibodies inhibit viral entry and/or replication.

w. Monoclonal Antibody COV49-33

In some embodiments, the antibody or antigen binding fragment is based on or derived from the COV49-33 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-33 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 177, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 181, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 177 and 181, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 178, 179, and 180, respectively, and/or a V_{L} comprising a LCDR1, a LCDR2, and a LCDR3 as set forth as SEQ ID NOs: 182, 183, and 184, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 178, 179, and 180, respectively, a V_{L} comprising a LCDR1, a LCDR2, and a LCDR3 as set forth as SEQ ID NOs: 182, 183, and 184, respectively, wherein the V_{H} comprises an amino acid sequence at least 90% identical to SEQ ID NO: 177, such as 95%, 96%, 97%, 98% or 99% identical to SEQ ID NO: 177, and wherein the V_{L} comprises an amino acid sequence at least 90% identical to SEQ ID NO: 181, such as 95%, 96%, 97%, 98% or 99% identical to SEQ ID NO: 181, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 177, and specifically binds to a coronavirus spike protein, and neutralizes a coronavirus. In more embodiments, the antibody or antigen binding fragment comprises a V_{L} comprising the amino acid sequence set forth as SEQ ID NO: 181, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 177 and 181, respectively, and specifically binds to a coronavirus spike protein, and neutralizes a coronavirus. The coronavirus can be SARS-CoV-2.

In some embodiments, the disclosed antibodies inhibit viral entry and/or replication.

x. Monoclonal Antibody COV49-42

In some embodiments, the antibody or antigen binding fragment is based on or derived from the COV49-42 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-42 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 185, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 189, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 185 and 189, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 186, 187, and 188, respectively, and/or a V_{L} comprising a LCDR1, a LCDR2, and a LCDR3 as set forth as SEQ ID NOs: 190, 191, 192, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 186, 187, and 188, respectively, a V_{L} comprising a LCDR1, a LCDR2, and a LCDR3 as set forth as SEQ ID NOs: 190, 191, 192, respectively, wherein the V_{H} comprises an amino acid sequence at least 90% identical to SEQ ID NO: 185, such as 95%, 96%, 97%, 98% or 99% identical to SEQ ID NO: 185, and wherein the V_{L} comprises an amino acid sequence at least 90% identical to SEQ ID NO: 189, such as 95%, 96%, 97%, 98% or 99% identical to SEQ ID NO: 189, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 185, and specifically binds to a coronavirus spike protein, and neutralizes a coronavirus. In more embodiments, the antibody or antigen binding fragment comprises a V_{L} comprising the amino acid sequence set forth as SEQ ID NO: 189, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 185 and 189, respectively, and specifically binds to a coronavirus spike protein, and neutralizes a coronavirus. The coronavirus can be SARS-CoV-2.

In some embodiments, the disclosed antibodies inhibit viral entry and/or replication.

y. Monoclonal Antibody COV49-47

In some embodiments, the antibody or antigen binding fragment is based on or derived from the COV49-47 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-47 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 193, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 197, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 193 and 197, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 194, 195, and 196, respectively, and/or a V_{L} comprising a LCDR1, a LCDR2, and a LCDR3 as set forth as SEQ ID NOs: 198, 199, and 200, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 194, 195, and 196, respectively, a V_{L} comprising a LCDR1, a LCDR2, and a LCDR3 as set forth as SEQ ID NOs: 198, 199, and 200, respectively, wherein the V_{H} comprises an amino acid sequence at least 90% identical to SEQ ID NO: 193, such as 95%, 96%, 97%, 98% or 99% identical to SEQ ID NO: 193, and wherein the V_{L} comprises an amino acid sequence at least 90% identical to SEQ ID NO: 197, such as 95%, 96%, 97%, 98% or 99% identical to SEQ ID NO: 197, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 193, and specifically binds to a coronavirus spike protein, and neutralizes a coronavirus. In more embodiments, the antibody or antigen binding fragment comprises a V_{L} comprising the amino acid sequence set forth as SEQ ID NO: 197, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 193 and 197, respectively, and specifically binds to a coronavirus spike protein, and neutralizes a coronavirus. The coronavirus can be SARS-CoV-2.

In some embodiments, the disclosed antibodies inhibit viral entry and/or replication.

z. Monoclonal Antibody COV49-54

In some embodiments, the antibody or antigen binding fragment is based on or derived from the COV49-54 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV49-54 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 201, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 205, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 201 and 205, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 202, 203, and 204, respectively, and/or a V_{L} comprising a LCDR1, a LCDR2, and a LCDR3 as set forth as SEQ ID NOs: 206, 207, and 208, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 202, 203, and 204, respectively, a V_{L} comprising a LCDR1, a LCDR2, and a LCDR3 as set forth as SEQ ID NOs: 206, 207, and 208, respectively, wherein the V_{H} comprises an amino acid sequence at least 90% identical to SEQ ID NO: 201, such as 95%, 96%, 97%, 98% or 99% identical to SEQ ID NO: 201, and wherein the V_{L} comprises an amino acid sequence at least 90% identical to SEQ ID NO: 205, such as 95%, 96%, 97%, 98% or 99% identical to SEQ ID NO: 205, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 201, and specifically binds to a coronavirus spike protein, and neutralizes a coronavirus. In more embodiments, the antibody or antigen binding fragment comprises a V_{L} comprising the amino acid sequence set forth as SEQ ID NO: 205, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 201 and 205, respectively, and specifically binds to a coronavirus spike protein, and neutralizes a coronavirus. The coronavirus can be SARS-CoV-2.

In some embodiments, the disclosed antibodies inhibit viral entry and/or replication.

aa. Monoclonal Antibody COV57-01

In some embodiments, the antibody or antigen binding fragment is based on or derived from the COV57-01 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV57-01 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 209, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 213, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 209 and 213, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 210, 211, and 212, respectively, and/or a V_{L} comprising a LCDR1, a LCDR2, and a LCDR3 as set forth as SEQ ID NOs: 214, 215, and 216, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 210, 211, and 212, respectively, a V_{L} comprising a LCDR1, a LCDR2, and a LCDR3 as set forth as SEQ ID NOs: 214, 215, and 216, respectively, wherein the V_{H} comprises an amino acid sequence at least 90% identical to SEQ ID NO: 209, such as 95%, 96%, 97%, 98% or 99% identical to SEQ ID NO: 209, and wherein the V_{L} comprises an amino acid sequence at least 90% identical to SEQ ID NO: 213, such as 95%, 96%, 97%, 98% or 99% identical to SEQ ID NO: 213, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 209, and specifically binds to a coronavirus spike protein, and neutralizes a coronavirus. In more embodiments, the antibody or antigen binding fragment comprises a V_{L} comprising the amino acid sequence set forth as SEQ ID NO: 213, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 209 and 213, respectively, and specifically binds to a coronavirus spike protein, and neutralizes a coronavirus. The coronavirus can be SARS-CoV-2.

In some embodiments, the disclosed antibodies inhibit viral entry and/or replication.

bb. Monoclonal Antibody COV57-03

In some embodiments, the antibody or antigen binding fragment is based on or derived from the COV57-03 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV57-03 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 217, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 221, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 217 and 221, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 218, 219, and 220, respectively, and/or a V_{L} comprising a LCDR1, a LCDR2, and a LCDR3 as set forth as SEQ ID NOs: 222, 223, and 224, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 218, 219, and 220, respectively, a V_{L} comprising a LCDR1, a LCDR2, and a LCDR3 as set forth as SEQ ID NOs: 222, 223, and 224, respectively, wherein the V_{H} comprises an amino acid sequence at least 90% identical to SEQ ID NO: 217, such as 95%, 96%, 97%, 98% or 99% identical to SEQ ID NO: 217, and wherein the V_{L} comprises an amino acid sequence at least 90% identical to SEQ ID NO: 221, such as 95%, 96%, 97%, 98% or 99% identical to SEQ ID NO: 221, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 217, and specifically binds to a coronavirus spike protein, and neutralizes a coronavirus. In more embodiments, the antibody or antigen binding fragment comprises a V_{L} comprising the amino acid sequence set forth as SEQ ID NO: 221, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 217 and 221, respectively, and specifically binds to a coronavirus spike protein, and neutralizes a coronavirus. The coronavirus can be SARS-CoV-2.

In some embodiments, the disclosed antibodies inhibit viral entry and/or replication.

cc. Monoclonal Antibody COV57-04

In some embodiments, the antibody or antigen binding fragment is based on or derived from the COV57-04 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV57-04 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 225, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 229, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 225 and 229, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 226, 227, and 228, respectively, and/or a V_{L} comprising a LCDR1, a LCDR2, and a LCDR3 as set forth as SEQ ID NOs: 230, 231, and 232, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 226, 227, and 228, respectively, a V_{L} comprising a LCDR1, a LCDR2, and a LCDR3 as set forth as SEQ ID NOs: 230, 231, and 232, respectively, wherein the V_{H} comprises an amino acid sequence at least 90% identical to SEQ ID NO: 225, such as 95%, 96%, 97%, 98% or 99% identical to SEQ ID NO: 225, and wherein the V_{L} comprises an amino acid sequence at least 90% identical to SEQ ID NO: 229, such as 95%, 96%, 97%, 98% or 99% identical to SEQ ID NO: 229, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 225, and specifically binds to a coronavirus spike protein, and neutralizes a coronavirus. In more embodiments, the antibody or antigen binding fragment comprises a V_{L} comprising the amino acid sequence set forth as SEQ ID NO: 229, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 225 and 229, respectively, and specifically binds to a coronavirus spike protein, and neutralizes a coronavirus. The coronavirus can be SARS-CoV-2.

In some embodiments, the disclosed antibodies inhibit viral entry and/or replication.

dd. Monoclonal Antibody COV57-05

In some embodiments, the antibody or antigen binding fragment is based on or derived from the COV57-05 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV57-05 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 233, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 237, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 233 and 237, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 234, 235, and 236, respectively, and/or a V_{L} comprising a LCDR1, a LCDR2, and a LCDR3 as set forth as SEQ ID NOs: 238, 239, and 240, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 234, 235, and 236, respectively, a V_{L} comprising a LCDR1, a LCDR2, and a LCDR3 as set forth as SEQ ID NOs: 238, 239, and 240, respectively, wherein the V_{H} comprises an amino acid sequence at least 90% identical to SEQ ID NO: 233, such as 95%, 96%, 97%, 98% or 99% identical to SEQ ID NO: 233, and wherein the V_{L} comprises an amino acid sequence at least 90% identical to SEQ ID NO: 237, such as 95%, 96%, 97%, 98% or 99% identical to SEQ ID NO: 237, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 233, and specifically binds to a coronavirus spike protein, and neutralizes a coronavirus. In more embodiments, the antibody or antigen binding fragment comprises a V_{L} comprising the amino acid sequence set forth as SEQ ID NO: 237, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 233 and 237, respectively, and specifically binds to a coronavirus spike protein, and neutralizes a coronavirus. The coronavirus can be SARS-CoV-2.

In some embodiments, the disclosed antibodies inhibit viral entry and/or replication.

ee. Monoclonal Antibody COV57-13

In some embodiments, the antibody or antigen binding fragment is based on or derived from the COV57-13 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV57-13 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 241, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 245, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 241 and 245, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 242, 243, and 244, respectively, and/or a V_{L} comprising a LCDR1, a LCDR2, and a LCDR3 as set forth as SEQ ID NOs: 246, 247, and 248, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 242, 243, and 244, respectively, a V_{L} comprising a LCDR1, a LCDR2, and a LCDR3 as set forth as SEQ ID NOs: 246, 247, and 248, respectively, wherein the V_{H} comprises an amino acid sequence at least 90% identical to SEQ ID NO: 241, such as 95%, 96%, 97%, 98% or 99% identical to SEQ ID NO: 241, and wherein the V_{L} comprises an amino acid sequence at least 90% identical to SEQ ID NO: 245, such as 95%, 96%, 97%, 98% or 99% identical to SEQ ID NO: 245, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 241, and specifically binds to a coronavirus spike protein, and neutralizes a coronavirus. In more embodiments, the antibody or antigen binding fragment comprises a V_{L} comprising the amino acid sequence set forth as SEQ ID NO: 245, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 241 and 245, respectively, and specifically binds to a coronavirus spike protein, and neutralizes a coronavirus. The coronavirus can be SARS-CoV-2.

In some embodiments, the disclosed antibodies inhibit viral entry and/or replication.

ff. Monoclonal Antibody COV57-19

In some embodiments, the antibody or antigen binding fragment is based on or derived from the COV57-19 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV57-19 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 249, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 253, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 249 and 253, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 250, 251, and 252, respectively, and/or a V_{L} comprising a LCDR1, a LCDR2, and a LCDR3 as set forth as SEQ ID NOs: 254, 255, and 256, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 250, 251, and 252, respectively, a V_{L} comprising a LCDR1, a LCDR2, and a LCDR3 as set forth as SEQ ID NOs: 254, 255, and 256, respectively, wherein the V_{H} comprises an amino acid sequence at least 90% identical to SEQ ID NO: 249, such as 95%, 96%, 97%, 98% or 99% identical to SEQ ID NO: 249, and wherein the V_{L} comprises an amino acid sequence at least 90% identical to SEQ ID NO: 253, such as 95%, 96%, 97%, 98% or 99% identical to SEQ ID NO: 253, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 249, and specifically binds to a coronavirus spike protein, and neutralizes a coronavirus. In more embodiments, the antibody or antigen binding fragment comprises a V_{L} comprising the amino acid sequence set forth as SEQ ID NO: 253, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 249 and 253, respectively, and specifically binds to a coronavirus spike protein, and neutralizes a coronavirus. The coronavirus can be SARS-CoV-2.

In some embodiments, the disclosed antibodies inhibit viral entry and/or replication.

gg. Monoclonal Antibody COV57-34

In some embodiments, the antibody or antigen binding fragment is based on or derived from the COV57-34 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV57-34 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 257, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 261, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 257 and 261, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 258, 259, and 260, respectively, and/or a V_{L} comprising a LCDR1, a LCDR2, and a LCDR3 as set forth as SEQ ID NOs: 262, 263, and 264, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 258, 259, and 260, respectively, a V_{L} comprising a LCDR1, a LCDR2, and a LCDR3 as set forth as SEQ ID NOs: 262, 263, and 264, respectively, wherein the V_{H} comprises an amino acid sequence at least 90% identical to SEQ ID NO: 257, such as 95%, 96%, 97%, 98% or 99% identical to SEQ ID NO: 257, and wherein the V_{L} comprises an amino acid sequence at least 90% identical to SEQ ID NO: 261, such as 95%, 96%, 97%, 98% or 99% identical to SEQ ID NO: 261, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 257, and specifically binds to a coronavirus spike protein, and neutralizes a coronavirus. In more embodiments, the antibody or antigen binding fragment comprises a V_{L} comprising the amino acid sequence set forth as SEQ ID NO: 261, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 257 and 261, respectively, and specifically binds to a coronavirus spike protein, and neutralizes a coronavirus. The coronavirus can be SARS-CoV-2.

In some embodiments, the disclosed antibodies inhibit viral entry and/or replication.

hh. Monoclonal Antibody COV57-38

In some embodiments, the antibody or antigen binding fragment is based on or derived from the COV57-38 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV57-38 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 265, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 269 and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 265 and 269, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 266, 267, and 268, respectively, and/or a V_{L} comprising a LCDR1, a LCDR2, and a LCDR3 as set forth as SEQ ID NOs: 270, 271, and 272, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 266, 267, and 268, respectively, a V_{L} comprising a LCDR1, a LCDR2, and a LCDR3 as set forth as SEQ ID NOs: 270, 271, and 272, respectively, wherein the V_{H} comprises an amino acid sequence at least 90% identical to SEQ ID NO: 265, such as 95%, 96%, 97%, 98% or 99% identical to SEQ ID NO: 265, and wherein the V_{L} comprises an amino acid sequence at least 90% identical to SEQ ID NO: 269, such as 95%, 96%, 97%, 98% or 99% identical to SEQ ID NO: 269, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 265, and specifically binds to a coronavirus spike protein, and neutralizes a coronavirus. In more embodiments, the antibody or antigen binding fragment comprises a V_{L} comprising the amino acid sequence set forth as SEQ ID NO: 269, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 265 and 269, respectively, and specifically binds to a coronavirus spike protein, and neutralizes a coronavirus. The coronavirus can be SARS-CoV-2.

In some embodiments, the disclosed antibodies inhibit viral entry and/or replication.

ii. Monoclonal Antibody COV57-45

In some embodiments, the antibody or antigen binding fragment is based on or derived from the COV57-45 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV57-45 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 273, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 277, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 273 and 277, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 274, 275, and 276, respectively, and/or a V_{L} comprising a LCDR1, a LCDR2, and a LCDR3 as set forth as SEQ ID NOs: 278, 279, and 280, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 274, 275, and 276, respectively, a V_{L} comprising a LCDR1, a LCDR2, and a LCDR3 as set forth as SEQ ID NOs: 278, 279, and 280, respectively, wherein the V_{H} comprises an amino acid sequence at least 90% identical to SEQ ID NO: 273, such as 95%, 96%, 97%, 98% or 99% identical to SEQ ID NO: 273, and wherein the V_{L} comprises an amino acid sequence at least 90% identical to SEQ ID NO: 277, such as 95%, 96%, 97%, 98% or 99% identical to SEQ ID NO: 277, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 273, and specifically binds to a coronavirus spike protein, and neutralizes a coronavirus. In more embodiments, the antibody or antigen binding fragment comprises a V_{L} comprising the amino acid sequence set forth as SEQ ID NO: 277, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 273 and 277, respectively, and specifically binds to a coronavirus spike protein, and neutralizes a coronavirus. The coronavirus can be SARS-CoV-2.

In some embodiments, the disclosed antibodies inhibit viral entry and/or replication.

jj. Monoclonal Antibody COV77-02

In some embodiments, the antibody or antigen binding fragment is based on or derived from the COV77-02 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-02 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 281, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 285, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 281 and 285, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 282, 283, and 284, respectively, and/or a V_{L} comprising a LCDR1, a LCDR2, and a LCDR3 as set forth as SEQ ID NOs: 286, 287, and 288, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 282, 283, and 284, respectively, a V_{L} comprising a LCDR1, a LCDR2, and a LCDR3 as set forth as SEQ ID NOs: 286, 287, and 288, respectively, wherein the V_{H} comprises an amino acid sequence at least 90% identical to SEQ ID NO: 281, such as 95%, 96%, 97%, 98% or 99% identical to SEQ ID NO: 281, and wherein the V_{L} comprises an amino acid sequence at least 90% identical to SEQ ID NO: 285, such as 95%, 96%, 97%, 98% or 99% identical to SEQ ID NO: 285, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 281, and specifically binds to a coronavirus spike protein, and neutralizes a coronavirus. In more embodiments, the antibody or antigen binding fragment comprises a V_{L} comprising the amino acid sequence set forth as SEQ ID NO: 285, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 281 and 285, respectively, and specifically binds to a coronavirus spike protein, and neutralizes a coronavirus. The coronavirus can be SARS-CoV-2.

In some embodiments, the disclosed antibodies inhibit viral entry and/or replication.

kk. Monoclonal Antibody COV77-04

In some embodiments, the antibody or antigen binding fragment is based on or derived from the COV77-04 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-04 antibody, and specifically binds to a coronavirus spike protein, and neutralizes a coronavirus. The coronavirus can be SARS-CoV-2. In some embodiments, the antibody or antigen binding fragment specifically binds a spike protein from SARS-CoV-2, SARS-CoV, MERS-CoV, HKU1, OC43, NL63 and 229E.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 289, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 293, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 289 and 293, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 290, 291, and 292 respectively, and/or a V_{L} comprising a LCDR1, a LCDR2, and a LCDR3 as set forth as SEQ ID NOs: 294, 295, and 296, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 290, 291, and 292, respectively, a V_{L} comprising a LCDR1, a LCDR2, and a LCDR3 as set forth as SEQ ID NOs: 294, 295, and 296, respectively, wherein the V_{H} comprises an amino acid sequence at least 90% identical to SEQ ID NO: 289, such as 95%, 96%, 97%, 98% or 99% identical to SEQ ID NO: 289, and wherein the V_{L} comprises an amino acid sequence at least 90% identical to SEQ ID NO: 293, such as 95%, 96%, 97%, 98% or 99% identical to SEQ ID NO: 293, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 289, and specifically binds to a coronavirus spike protein, and neutralizes a coronavirus. In more embodiments, the antibody or antigen binding fragment comprises a V_{L} comprising the amino acid sequence set forth as SEQ ID NO: 293, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 289 and 293, respectively, and specifically binds to a coronavirus spike protein, and neutralizes a coronavirus. The coronavirus can be SARS-CoV-2.

In some embodiments, the disclosed antibodies inhibit viral entry and/or replication.

ll. Monoclonal Antibody COV77-05

In some embodiments, the antibody or antigen binding fragment is based on or derived from the COV77-05 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-05 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 297, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 301, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 297 and 301, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 298, 299, and 300, respectively, and/or a V_{L} comprising a LCDR1, a LCDR2, and a LCDR3 as set forth as SEQ ID NOs: 302, 303, and 304, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 298, 299, and 300, respectively, a V_{L} comprising a LCDR1, a LCDR2, and a LCDR3 as set forth as SEQ ID NOs: 302, 303, and 304, respectively, wherein the V_{H} comprises an amino acid sequence at least 90% identical to SEQ ID NO: 297, such as 95%, 96%, 97%, 98% or 99% identical to SEQ ID NO: 297, and wherein the V_{L} comprises an amino acid sequence at least 90% identical to SEQ ID NO: 301, such as 95%, 96%, 97%, 98% or 99% identical to SEQ ID NO: 301, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 297, and specifically binds to a coronavirus spike protein, and neutralizes a coronavirus. In more embodiments, the antibody or antigen binding fragment comprises a V_{L} comprising the amino acid sequence set forth as SEQ ID NO: 301, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 297 and 301, respectively, and specifically binds to a coronavirus spike protein, and neutralizes a coronavirus. The coronavirus can be SARS-CoV-2.

In some embodiments, the disclosed antibodies inhibit viral entry and/or replication.

mm. Monoclonal Antibody COV77-09

In some embodiments, the antibody or antigen binding fragment is based on or derived from the COV77-09 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-09 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 305, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 309, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 305 and 309, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 306, 307, and 308, respectively, and/or a V_{L} comprising a LCDR1, a LCDR2, and a LCDR3 as set forth as SEQ ID NOs: 310, 311, and 312, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 306, 307, and 308, respectively, a V_{L} comprising a LCDR1, a LCDR2, and a LCDR3 as set forth as SEQ ID NOs: 310, 311, and 312, respectively, wherein the V_{H} comprises an amino acid sequence at least 90% identical to SEQ ID NO: 305, such as 95%, 96%, 97%, 98% or 99% identical to SEQ ID NO: 305, and wherein the V_{L} comprises an amino acid sequence at least 90% identical to SEQ ID NO: 309, such as 95%, 96%, 97%, 98% or 99% identical to SEQ ID NO: 309, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 305, and specifically binds to a coronavirus spike protein, and neutralizes a coronavirus. In more embodiments, the antibody or antigen binding fragment comprises a V_{L} comprising the amino acid sequence set forth as SEQ ID NO: 309, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 305 and 309, respectively, and specifically binds to a coronavirus spike protein, and neutralizes a coronavirus. The coronavirus can be SARS-CoV-2.

In some embodiments, the disclosed antibodies inhibit viral entry and/or replication.

nn. Monoclonal Antibody COV77-14

In some embodiments, the antibody or antigen binding fragment is based on or derived from the COV77-14 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-14 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 313, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 317, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 313 and 317, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 314, 315, and 316, respectively, and/or a V_{L} comprising a LCDR1, a LCDR2, and a LCDR3 as set forth as SEQ ID NOs: 318, 319, and 320, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 314, 315, and 316, respectively, a V_{L} comprising a LCDR1, a LCDR2, and a LCDR3 as set forth as SEQ ID NOs: 318, 319, and 320, respectively, wherein the V_{H} comprises an amino acid sequence at least 90% identical to SEQ ID NO: 313, such as 95%, 96%, 97%, 98% or 99% identical to SEQ ID NO: 313, and wherein the V_{L} comprises an amino acid sequence at least 90% identical to SEQ ID NO: 317, such as 95%, 96%, 97%, 98% or 99% identical to SEQ ID NO: 317, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 313, and specifically binds to a coronavirus spike protein, and neutralizes a coronavirus. In more embodiments, the antibody or antigen binding fragment comprises a V_{L} comprising the amino acid sequence set forth as SEQ ID NO: 317, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 313 and 317, respectively, and specifically binds to a coronavirus spike protein, and neutralizes a coronavirus. The coronavirus can be SARS-CoV-2.

In some embodiments, the disclosed antibodies inhibit viral entry and/or replication.

oo. Monoclonal Antibody COV77-35

In some embodiments, the antibody or antigen binding fragment is based on or derived from the COV77-35 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-35 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 321, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 325, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 321 and 325, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 322, 323, and 324, respectively, and/or a V_{L} comprising a LCDR1, a LCDR2, and a LCDR3 as set forth as SEQ ID NOs: 326, 327, and 328, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 322, 323, and 324, respectively, a V_{L} comprising a LCDR1, a LCDR2, and a LCDR3 as set forth as SEQ ID NOs: 326, 327, and 328, respectively, wherein the V_{H} comprises an amino acid sequence at least 90% identical to SEQ ID NO: 321, such as 95%, 96%, 97%, 98% or 99% identical to SEQ ID NO: 321, and wherein the V_{L} comprises an amino acid sequence at least 90% identical to SEQ ID NO: 325, such as 95%, 96%, 97%, 98% or 99% identical to SEQ ID NO: 325, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 321, and specifically binds to a coronavirus spike protein, and neutralizes a coronavirus. In more embodiments, the antibody or antigen binding fragment comprises a V_{L} comprising the amino acid sequence set forth as SEQ ID NO: 325, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 321 and 325, respectively, and specifically binds to a coronavirus spike protein, and neutralizes a coronavirus. The coronavirus can be SARS-CoV-2.

In some embodiments, the disclosed antibodies inhibit viral entry and/or replication.

pp. Monoclonal Antibody COV77-39

In some embodiments, the antibody or antigen binding fragment is based on or derived from the COV77-39 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-39 antibody, and specifically binds to a coronavirus spike protein, and neutralizes a coronavirus. The coronavirus can be SARS-CoV-2. In some embodiments, the antibody or antigen binding fragment specifically binds a spike protein from SARS-CoV-2, SARS-CoV, MERS-CoV, HKU1, OC43, NL63 and 229E.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 329, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 333, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 329 and 333, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 330, 331, and 332, respectively, and/or a V_{L} comprising a LCDR1, a LCDR2, and a LCDR3 as set forth as SEQ ID NOs: 334, 335, and 336, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 330, 331, and 332, respectively, a V_{L} comprising a LCDR1, a LCDR2, and a LCDR3 as set forth as SEQ ID NOs: 334, 335, and 336, respectively, wherein the V_{H} comprises an amino acid sequence at least 90% identical to SEQ ID NO: 329, such as 95%, 96%, 97%, 98% or 99% identical to SEQ ID NO: 329, and wherein the V_{L} comprises an amino acid sequence at least 90% identical to SEQ ID NO: 333, such as 95%, 96%, 97%, 98% or 99% identical to SEQ ID NO: 333, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 329, and specifically binds to a coronavirus spike protein, and neutralizes a coronavirus. In more embodiments, the antibody or antigen binding fragment comprises a V_{L} comprising the amino acid sequence set forth as SEQ ID NO: 333, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 329 and 333, respectively, and specifically binds to a coronavirus spike protein, and neutralizes a coronavirus. The coronavirus can be SARS-CoV-2.

In some embodiments, the disclosed antibodies inhibit viral entry and/or replication.

qq. Monoclonal Antibody COV77-42

In some embodiments, the antibody or antigen binding fragment is based on or derived from the COV77-42 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-42 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 337, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 341, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 337 and 341, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 338, 339, and 340, respectively, and/or a V_{L} comprising a LCDR1, a LCDR2, and a LCDR3 as set forth as SEQ ID NOs: 342, 343, and 344, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 338, 339, and 340, respectively, a V_{L} comprising a LCDR1, a LCDR2, and a LCDR3 as set forth as SEQ ID NOs: 342, 343, and 344, respectively, wherein the V_{H} comprises an amino acid sequence at least 90% identical to SEQ ID NO: 337, such as 95%, 96%, 97%, 98% or 99% identical to SEQ ID NO: 337, and wherein the V_{L} comprises an amino acid sequence at least 90% identical to SEQ ID NO: 341, such as 95%, 96%, 97%, 98% or 99% identical to SEQ ID NO: 341, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 337, and specifically binds to a coronavirus spike protein, and neutralizes a coronavirus. In more embodiments, the antibody or antigen binding fragment comprises a V_{L} comprising the amino acid sequence set forth as SEQ ID NO: 341, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 337 and 341, respectively, and specifically binds to a coronavirus spike protein, and neutralizes a coronavirus. The coronavirus can be SARS-CoV-2.

In some embodiments, the disclosed antibodies inhibit viral entry and/or replication.

rr. Monoclonal Antibody COV77-43

In some embodiments, the antibody or antigen binding fragment is based on or derived from the COV77-43 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-43 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 345, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 349, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 345 and 349, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 346, 347, and 348, respectively, and/or a V_{L} comprising a LCDR1, a LCDR2, and a LCDR3 as set forth as SEQ ID NOs: 350, 351, and 352, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 346, 347, and 348, respectively, a V_{L} comprising a LCDR1, a LCDR2, and a LCDR3 as set forth as SEQ ID NOs: 350, 351, and 352, respectively, wherein the V_{H} comprises an amino acid sequence at least 90% identical to SEQ ID NO: 345, such as 95%, 96%, 97%, 98% or 99% identical to SEQ ID NO: 345, and wherein the V_{L} comprises an amino acid sequence at least 90% identical to SEQ ID NO: 349, such as 95%, 96%, 97%, 98% or 99% identical to SEQ ID NO: 349, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 345, and specifically binds to a coronavirus spike protein, and neutralizes a coronavirus. In more embodiments, the antibody or antigen binding fragment comprises a V_{L} comprising the amino acid sequence set forth as SEQ ID NO: 349, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 345 and 349, respectively, and specifically binds to a coronavirus spike protein, and neutralizes a coronavirus. The coronavirus can be SARS-CoV-2.

In some embodiments, the disclosed antibodies inhibit viral entry and/or replication.

ss. Monoclonal Antibody COV77-46

In some embodiments, the antibody or antigen binding fragment is based on or derived from the COV77-46 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-46 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 353, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 357, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 353 and 357, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 354, 355, and 356, respectively, and/or a V_{L} comprising a LCDR1, a LCDR2, and a LCDR3 as set forth as SEQ ID NOs: 358, 359, and 360, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 354, 355, and 356, respectively, a V_{L} comprising a LCDR1, a LCDR2, and a LCDR3 as set forth as SEQ ID NOs: 358, 359, and 360, respectively, wherein the V_{H} comprises an amino acid sequence at least 90% identical to SEQ ID NO: 353, such as 95%, 96%, 97%, 98% or 99% identical to SEQ ID NO: 353, and wherein the V_{L} comprises an amino acid sequence at least 90% identical to SEQ ID NO: 357, such as 95%, 96%, 97%, 98% or 99% identical to SEQ ID NO: 357, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 353, and specifically binds to a coronavirus spike protein, and neutralizes a coronavirus. In more embodiments, the antibody or antigen binding fragment comprises a V_{L} comprising the amino acid sequence set forth as SEQ ID NO: 357, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 353 and 357, respectively, and specifically binds to a coronavirus spike protein, and neutralizes a coronavirus. The coronavirus can be SARS-CoV-2.

In some embodiments, the disclosed antibodies inhibit viral entry and/or replication.

tt. Monoclonal Antibody COV77-76

In some embodiments, the antibody or antigen binding fragment is based on or derived from the COV77-76 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV77-76 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 361, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 365, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 361 and 365, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 362, 363, and 364, respectively, and/or a V_{L} comprising a LCDR1, a LCDR2, and a LCDR3 as set forth as SEQ ID NOs: 366, 367, and 368, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 362, 363, and 364, respectively, a V_{L} comprising a LCDR1, a LCDR2, and a LCDR3 as set forth as SEQ ID NOs: 366, 367, and 368, respectively, wherein the V_{H} comprises an amino acid sequence at least 90% identical to SEQ ID NO: 361, such as 95%, 96%, 97%, 98% or 99% identical to SEQ ID NO: 361, and wherein the V_{L} comprises an amino acid sequence at least 90% identical to SEQ ID NO: 365, such as 95%, 96%, 97%, 98% or 99% identical to SEQ ID NO: 365, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 361, and specifically binds to a coronavirus spike protein, and neutralizes a coronavirus. In more embodiments, the antibody or antigen binding fragment comprises a V_{L} comprising the amino acid sequence set forth as SEQ ID NO: 365, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 361 and 365, respectively, and specifically binds to a coronavirus spike protein, and neutralizes a coronavirus. The coronavirus can be SARS-CoV-2.

In some embodiments, the disclosed antibodies inhibit viral entry and/or replication.

uu. Monoclonal Antibody COV93-04 In some embodiments, the antibody or antigen binding fragment is based on or derived from the COV93-04 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV93-04 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 369, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 373, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 369 and 373, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 370, 371, and 372, respectively, and/or a V_{L} comprising a LCDR1, a LCDR2, and a LCDR3 as set forth as SEQ ID NOs: 374, 375, and 376, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 370, 371, and 372, respectively, a V_{L} comprising a LCDR1, a LCDR2, and a LCDR3 as set forth as SEQ ID NOs: 374, 375, and 376, respectively, wherein the V_{H} comprises an amino acid sequence at least 90% identical to SEQ ID NO: 369, such as 95%, 96%, 97%, 98% or 99% identical to SEQ ID NO: 369, and wherein the V_{L} comprises an amino acid sequence at least 90% identical to SEQ ID NO: 373, such as 95%, 96%, 97%, 98% or 99% identical to SEQ ID NO: 373, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 369, and specifically binds to a coronavirus spike protein, and neutralizes a coronavirus. In more embodiments, the antibody or antigen binding fragment comprises a V_{L} comprising the amino acid sequence set forth as SEQ ID NO: 373, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 369 and 373, respectively, and specifically binds to a coronavirus spike protein, and neutralizes a coronavirus. The coronavirus can be SARS-CoV-2.

In some embodiments, the disclosed antibodies inhibit viral entry and/or replication.

vv. Monoclonal Antibody COV93-08

In some embodiments, the antibody or antigen binding fragment is based on or derived from the COV93-08 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV93-08 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 377, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 381, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 377 and 381, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 378, 379, and 380, respectively, and/or a V_{L} comprising a LCDR1, a LCDR2, and a LCDR3 as set forth as SEQ ID NOs: 382, 383, and 384, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 378, 379, and 380, respectively, a V_{L} comprising a LCDR1, a LCDR2, and a LCDR3 as set forth as SEQ ID NOs: 382, 383, and 384, respectively, wherein the V_{H} comprises an amino acid sequence at least 90% identical to SEQ ID NO: 377, such as 95%, 96%, 97%, 98% or 99% identical to SEQ ID NO: 377, and wherein the V_{L} comprises an amino acid sequence at least 90% identical to SEQ ID NO: 381, such as 95%, 96%, 97%, 98% or 99% identical to SEQ ID NO: 381, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 377, and specifically binds to a coronavirus spike protein, and neutralizes a coronavirus. In more embodiments, the antibody or antigen binding fragment comprises a V_{L} comprising the amino acid sequence set forth as SEQ ID NO: 381, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 377 and 381, respectively, and specifically binds to a coronavirus spike protein, and neutralizes a coronavirus. The coronavirus can be SARS-CoV-2.

In some embodiments, the disclosed antibodies inhibit viral entry and/or replication.

ww. Monoclonal Antibody COV93-17

In some embodiments, the antibody or antigen binding fragment is based on or derived from the COV93-17 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV93-17 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 385, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 389, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 385 and 389, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 386, 387, and 388, respectively, and/or a V_{L} comprising a LCDR1, a LCDR2, and a LCDR3 as set forth as SEQ ID NOs: 390, 391, and 392, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 386, 387, and 388, respectively, a V_{L} comprising a LCDR1, a LCDR2, and a LCDR3 as set forth as SEQ ID NOs: 390, 391, and 392, respectively, wherein the V_{H} comprises an amino acid sequence at least 90% identical to SEQ ID NO: 385, such as 95%, 96%, 97%, 98% or 99% identical to SEQ ID NO: 385, and wherein the V_{L} comprises an amino acid sequence at least 90% identical to SEQ ID NO: 389, such as 95%, 96%, 97%, 98% or 99% identical to SEQ ID NO: 389, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 385, and specifically binds to a coronavirus spike protein, and neutralizes a coronavirus. In more embodiments, the antibody or antigen binding fragment comprises a V_{L} comprising the amino acid sequence set forth as SEQ ID NO: 389, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 385 and 389, respectively, and specifically binds to a coronavirus spike protein, and neutralizes a coronavirus. The coronavirus can be SARS-CoV-2.

In some embodiments, the disclosed antibodies inhibit viral entry and/or replication.

xx. Monoclonal Antibody COV93-18

In some embodiments, the antibody or antigen binding fragment is based on or derived from the COV93-18 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV93-18 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 393, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 397, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 393 and 397, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 394, 395, and 396, respectively, and/or a V_{L} comprising a LCDR1, a LCDR2, and a LCDR3 as set forth as SEQ ID NOs: 398, 399, and 400, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 394, 395, and 396, respectively, a V_{L} comprising a LCDR1, a LCDR2, and a LCDR3 as set forth as SEQ ID NOs: 398, 399, and 400, respectively, wherein the V_{H} comprises an amino acid sequence at least 90% identical to SEQ ID NO: 393, such as 95%, 96%, 97%, 98% or 99% identical to SEQ ID NO: 393, and wherein the V_{L} comprises an amino acid sequence at least 90% identical to SEQ ID NO: 397, such as 95%, 96%, 97%, 98% or 99% identical to SEQ ID NO: 397, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 393, and specifically binds to a coronavirus spike protein, and neutralizes a coronavirus. In more embodiments, the antibody or antigen binding fragment comprises a V_{L} comprising the amino acid sequence set forth as SEQ ID NO: 397, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 393 and 397, respectively, and specifically binds to a coronavirus spike protein, and neutralizes a coronavirus. The coronavirus can be SARS-CoV-2.

In some embodiments, the disclosed antibodies inhibit viral entry and/or replication.

yy. Monoclonal Antibody COV93-23

In some embodiments, the antibody or antigen binding fragment is based on or derived from the COV93-23 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV93-23 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 401, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 405, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 401 and 405, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 402, 403, and 404, respectively, and/or a V_{L} comprising a LCDR1, a LCDR2, and a LCDR3 as set forth as SEQ ID NOs: 406, 407, and 408, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 402, 403, and 404, respectively, a V_{L} comprising a LCDR1, a LCDR2, and a LCDR3 as set forth as SEQ ID NOs: 406, 407, and 408, respectively, wherein the V_{H} comprises an amino acid sequence at least 90% identical to SEQ ID NO: 401, such as 95%, 96%, 97%, 98% or 99% identical to SEQ ID NO: 401, and wherein the V_{L} comprises an amino acid sequence at least 90% identical to SEQ ID NO: 405, such as 95%, 96%, 97%, 98% or 99% identical to SEQ ID NO: 405, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 401, and specifically binds to a coronavirus spike protein, and neutralizes a coronavirus. In more embodiments, the antibody or antigen binding fragment comprises a V_{L} comprising the amino acid sequence set forth as SEQ ID NO: 405, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 401 and 405, respectively, and specifically binds to a coronavirus spike protein, and neutralizes a coronavirus. The coronavirus can be SARS-CoV-2.

In some embodiments, the disclosed antibodies inhibit viral entry and/or replication.

zz. Monoclonal Antibody COV78-36

In some embodiments, the antibody or antigen binding fragment is based on or derived from the COV78-36 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV78-36 antibody, and specifically binds to a coronavirus spike protein, and neutralizes a coronavirus. The coronavirus can be SARS-CoV-2. In some embodiments, the antibody or antigen binding fragment specifically binds a spike protein from SARS-CoV-2, SARS-CoV, MERS-CoV, HKU1, OC43, NL63 and 229E.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 409, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 413, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 409 and 413, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 410, 411, and 412, respectively, and/or a V_{L} comprising a LCDR1, a LCDR2, and a LCDR3 as set forth as SEQ ID NOs: 414, 415, and 416, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 410, 411, and 412, respectively, a V_{L} comprising a LCDR1, a LCDR2, and a LCDR3 as set forth as SEQ ID NOs: 414, 415, and 416, respectively, wherein the V_{H} comprises an amino acid sequence at least 90% identical to SEQ ID NO: 409, such as 95%, 96%, 97%, 98% or 99% identical to SEQ ID NO: 409, and wherein the V_{L} comprises an amino acid sequence at least 90% identical to SEQ ID NO: 413, such as 95%, 96%, 97%, 98% or 99% identical to SEQ ID NO: 413, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 409, and specifically binds to a coronavirus spike protein, and neutralizes a coronavirus. In more embodiments, the antibody or antigen binding fragment comprises a V_{L} comprising the amino acid sequence set forth as SEQ ID NO: 413, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 409 and 413, respectively, and specifically binds to a coronavirus spike protein, and neutralizes a coronavirus. The coronavirus can be SARS-CoV-2.

In some embodiments, the disclosed antibodies inhibit viral entry and/or replication.

aaa. Monoclonal Antibody COV93-38 In some embodiments, the antibody or antigen binding fragment is based on or derived from the COV93-38 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV93-38 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 417, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 421, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 417 and 421, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 418, 419, and 420, respectively, and/or a V_{L} comprising a LCDR1, a LCDR2, and a LCDR3 as set forth as SEQ ID NOs: 422, 423, and 424, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 418, 419, and 420, respectively, a V_{L} comprising a LCDR1, a LCDR2, and a LCDR3 as set forth as SEQ ID NOs: 422, 423, and 424, respectively, wherein the V_{H} comprises an amino acid sequence at least 90% identical to SEQ ID NO: 417, such as 95%, 96%, 97%, 98% or 99% identical to SEQ ID NO: 417, and wherein the V_{L} comprises an amino acid sequence at least 90% identical to SEQ ID NO: 421, such as 95%, 96%, 97%, 98% or 99% identical to SEQ ID NO: 421, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 417, and specifically binds to a coronavirus spike protein, and neutralizes a coronavirus. In more embodiments, the antibody or antigen binding fragment comprises a V_{L} comprising the amino acid sequence set forth as SEQ ID NO: 421, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 417 and 421, respectively, and specifically binds to a coronavirus spike protein, and neutralizes a coronavirus. The coronavirus can be SARS-CoV-2.

In some embodiments, the disclosed antibodies inhibit viral entry and/or replication.

bbb. Monoclonal Antibody COV93-60

In some embodiments, the antibody or antigen binding fragment is based on or derived from the COV93-60 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV93-60 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 425, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 429, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 425 and 429, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 426, 427, and 428, respectively, and/or a V_{L} comprising a LCDR1, a LCDR2, and a LCDR3 as set forth as SEQ ID NOs: 430, 431, and 432, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 426, 427, and 428, respectively, a V_{L} comprising a LCDR1, a LCDR2, and a LCDR3 as set forth as SEQ ID NOs: 430, 431, and 432, respectively, wherein the V_{H} comprises an amino acid sequence at least 90% identical to SEQ ID NO: 425, such as 95%, 96%, 97%, 98% or 99% identical to SEQ ID NO: 425, and wherein the V_{L} comprises an amino acid sequence at least 90% identical to SEQ ID NO: 429, such as 95%, 96%, 97%, 98% or 99% identical to SEQ ID NO: 429, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 425, and specifically binds to a coronavirus spike protein, and neutralizes a coronavirus. In more embodiments, the antibody or antigen binding fragment comprises a V_{L} comprising the amino acid sequence set forth as SEQ ID NO: 429, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 425 and 429, respectively, and specifically binds to a coronavirus spike protein, and neutralizes a coronavirus. The coronavirus can be SARS-CoV-2.

In some embodiments, the disclosed antibodies inhibit viral entry and/or replication.

ccc. Monoclonal Antibody COV93-61

In some embodiments, the antibody or antigen binding fragment is based on or derived from the COV93-61 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV93-61 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 433, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 437, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 433 and 437, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 434, 435, and 436, respectively, and/or a V_{L} comprising a LCDR1, a LCDR2, and a LCDR3 as set forth as SEQ ID NOs: 438, 439, and 440, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 434, 435, and 436, respectively, a V_{L} comprising a LCDR1, a LCDR2, and a LCDR3 as set forth as SEQ ID NOs: 438, 439, and 440, respectively, wherein the V_{H} comprises an amino acid sequence at least 90% identical to SEQ ID NO: 433, such as 95%, 96%, 97%, 98% or 99% identical to SEQ ID NO: 433, and wherein the V_{L} comprises an amino acid sequence at least 90% identical to SEQ ID NO: 437, such as 95%, 96%, 97%, 98% or 99% identical to SEQ ID NO: 437, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 433, and specifically binds to a coronavirus spike protein, and neutralizes a coronavirus. In more embodiments, the antibody or antigen binding fragment comprises a V_{L} comprising the amino acid sequence set forth as SEQ ID NO: 437, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 433 and 437, respectively, and specifically binds to a coronavirus spike protein, and neutralizes a coronavirus. The coronavirus can be SARS-CoV-2.

In some embodiments, the disclosed antibodies inhibit viral entry and/or replication.

ddd. Monoclonal Antibody COV89-03

In some embodiments, the antibody or antigen binding fragment is based on or derived from the COV89-03 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV89-03 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 441, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 445, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 441 and 445, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 442, 443, and 444, respectively, and/or a V_{L} comprising a LCDR1, a LCDR2, and a LCDR3 as set forth as SEQ ID NOs: 446, 447, and 448, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 442, 443, and 444, respectively, a V_{L} comprising a LCDR1, a LCDR2, and a LCDR3 as set forth as SEQ ID NOs: 446, 447, and 448, respectively, wherein the V_{H} comprises an amino acid sequence at least 90% identical to SEQ ID NO: 441, such as 95%, 96%, 97%, 98% or 99% identical to SEQ ID NO: 441, and wherein the V_{L} comprises an amino acid sequence at least 90% identical to SEQ ID NO: 445, such as 95%, 96%, 97%, 98% or 99% identical to SEQ ID NO: 445, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 441, and specifically binds to a coronavirus spike protein, and neutralizes a coronavirus. In more embodiments, the antibody or antigen binding fragment comprises a V_{L} comprising the amino acid sequence set forth as SEQ ID NO: 445, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 441 and 445, respectively, and specifically binds to a coronavirus spike protein, and neutralizes a coronavirus. The coronavirus can be SARS-CoV-2.

In some embodiments, the disclosed antibodies inhibit viral entry and/or replication.

eee. Monoclonal Antibody COV89-28

In some embodiments, the antibody or antigen binding fragment is based on or derived from the COV89-28 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV89-28 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 449, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 453, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 449 and 453, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 450, 451, and 452, respectively, and/or a V_{L} comprising a LCDR1, a LCDR2, and a LCDR3 as set forth as SEQ ID NOs: 454, 455, and 456, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 450, 451, and 452, respectively, a V_{L} comprising a LCDR1, a LCDR2, and a LCDR3 as set forth as SEQ ID NOs: 454, 455, and 456, respectively, wherein the V_{H} comprises an amino acid sequence at least 90% identical to SEQ ID NO: 449, such as 95%, 96%, 97%, 98% or 99% identical to SEQ ID NO: 449, and wherein the V_{L} comprises an amino acid sequence at least 90% identical to SEQ ID NO: 453, such as 95%, 96%, 97%, 98% or 99% identical to SEQ ID NO: 453, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 449, and specifically binds to a coronavirus spike protein, and neutralizes a coronavirus. In more embodiments, the antibody or antigen binding fragment comprises a V_{L} comprising the amino acid sequence set forth as SEQ ID NO: 453, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 449 and 453, respectively, and specifically binds to a coronavirus spike protein, and neutralizes a coronavirus. The coronavirus can be SARS-CoV-2.

In some embodiments, the disclosed antibodies inhibit viral entry and/or replication.

fff. Monoclonal Antibody COV30-35

In some embodiments, the antibody or antigen binding fragment is based on or derived from the COV30-35 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV30-35 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 457, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 461, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 457 and 461, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 458, 459, and 460, respectively, and/or a V_{L} comprising a LCDR1, a LCDR2, and a LCDR3 as set forth as SEQ ID NOs: 462, 463 and 464, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 458, 459, and 460, respectively, a V_{L} comprising a LCDR1, a LCDR2, and a LCDR3 as set forth as SEQ ID NOs: 462, 463 and 464, respectively, wherein the V_{H} comprises an amino acid sequence at least 90% identical to SEQ ID NO: 457, such as 95%, 96%, 97%, 98% or 99% identical to SEQ ID NO: 457, and wherein the V_{L} comprises an amino acid sequence at least 90% identical to SEQ ID NO: 461, such as 95%, 96%, 97%, 98% or 99% identical to SEQ ID NO: 461, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 457, and specifically binds to a coronavirus spike protein, and neutralizes a coronavirus. In more embodiments, the antibody or antigen binding fragment comprises a V_{L} comprising the amino acid sequence set forth as SEQ ID NO: 461, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 457 and 461, respectively, and specifically binds to a coronavirus spike protein, and neutralizes a coronavirus. The coronavirus can be SARS-CoV-2.

In some embodiments, the disclosed antibodies inhibit viral entry and/or replication.

ggg. Monoclonal Antibody COV30-80

In some embodiments, the antibody or antigen binding fragment is based on or derived from the COV30-80 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV30-80 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 465, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 469, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 465 and 469, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 466, 467, and 468, respectively, and/or a V_{L} comprising a LCDR1, a LCDR2, and a LCDR3 as set forth as SEQ ID NOs: 470, 471, and 472, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 466, 467, and 468, respectively, a V_{L} comprising a LCDR1, a LCDR2, and a LCDR3 as set forth as SEQ ID NOs: 470, 471, and 472, respectively, wherein the V_{H} comprises an amino acid sequence at least 90% identical to SEQ ID NO: 465, such as 95%, 96%, 97%, 98% or 99% identical to SEQ ID NO: 465, and wherein the V_{L} comprises an amino acid sequence at least 90% identical to SEQ ID NO: 469, such as 95%, 96%, 97%, 98% or 99% identical to SEQ ID NO: 469, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 465, and specifically binds to a coronavirus spike protein, and neutralizes a coronavirus. In more embodiments, the antibody or antigen binding fragment comprises a V_{L} comprising the amino acid sequence set forth as SEQ ID NO: 469, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 465 and 469, respectively, and specifically binds to a coronavirus spike protein, and neutralizes a coronavirus. The coronavirus can be SARS-CoV-2.

In some embodiments, the disclosed antibodies inhibit viral entry and/or replication.

hhh. Monoclonal Antibody COV44-25

In some embodiments, the antibody or antigen binding fragment is based on or derived from the COV44-25 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV44-25 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 473, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 477, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 473 and 477, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 474, 475, and 476, respectively, and/or a V_{L} comprising a LCDR1, a LCDR2, and a LCDR3 as set forth as SEQ ID NOs: 478, 479, and 480, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 474, 475, and 476, respectively, a V_{L} comprising a LCDR1, a LCDR2, and a LCDR3 as set forth as SEQ ID NOs: 478, 479, and 480, respectively, wherein the V_{H} comprises an amino acid sequence at least 90% identical to SEQ ID NO: 473, such as 95%, 96%, 97%, 98% or 99% identical to SEQ ID NO: 473, and wherein the V_{L} comprises an amino acid sequence at least 90% identical to SEQ ID NO: 477, such as 95%, 96%, 97%, 98% or 99% identical to SEQ ID NO: 477, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 473, and specifically binds to a coronavirus spike protein, and neutralizes a coronavirus. In more embodiments, the antibody or antigen binding fragment comprises a V_{L} comprising the amino acid sequence set forth as SEQ ID NO: 477, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 473 and 477, respectively, and specifically binds to a coronavirus spike protein, and neutralizes a coronavirus. The coronavirus can be SARS-CoV-2.

In some embodiments, the disclosed antibodies inhibit viral entry and/or replication.

iii. Monoclonal Antibody COV44-37

In some embodiments, the antibody or antigen binding fragment is based on or derived from the COV44-37 antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the COV44-37 antibody, and specifically binds to a coronavirus spike protein, and neutralizes a coronavirus. The coronavirus can be SARS-CoV-2. In some embodiments, the antibody or antigen binding fragment specifically binds a spike protein from at least three betacoronaviruses selected from the group consisting of SARS-CoV-2, SARS-CoV, MERS-CoV, HKU1, and OC43. In some embodiments, the antibody or antigen binding fragment specifically binds a stem-helix in the S2 domain of the spike protein.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 481, and specifically binds to a coronavirus spike, and neutralizes a coronavirus. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 485, and specifically binds to a coronavirus spike protein, and neutralizes a coronavirus. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 481 and 485, respectively, and specifically binds to a coronavirus spike protein and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 482, 483, and 484, respectively, and/or a V_{L} comprising a LCDR1, a LCDR2, and a LCDR3 as set forth as SEQ ID NOs: 486, 487, and 488, respectively, and specifically binds to a coronavirus spike protein, and neutralizes a coronavirus. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 482, 483, and 484, respectively, a V_{L} comprising a LCDR1, a LCDR2, and a LCDR3 as set forth as SEQ ID NOs: 486, 487, and 488, respectively, wherein the V_{H} comprises an amino acid sequence at least 90% identical to SEQ ID NO: 481, such as 95%, 96%, 97%, 98% or 99% identical to SEQ ID NO: 481, and wherein the V_{L} comprises an amino acid sequence at least 90% identical to SEQ ID NO: 485, such as 95%, 96%, 97%, 98% or 99% identical to SEQ ID NO: 485, and the antibody or antigen binding fragment specifically binds to a coronavirus spike protein, and neutralizes a coronavirus. In this embodiment, variations due to sequence identify fall outside the CDRs. The coronavirus can be SARS-CoV-2.

In some embodiments, the antibody or antigen binding fragment comprises a V_{H} comprising the amino acid sequence set forth as SEQ ID NO: 481, and specifically binds to a coronavirus spike protein, and neutralizes a coronavirus. In more embodiments, the antibody or antigen binding fragment comprises a V_{L} comprising the amino acid sequence set forth as SEQ ID NO: 485, and specifically binds to a coronavirus spike protein, and neutralizes a coronavirus. In some embodiments, the antibody or antigen binding fragment comprises a V_{H} and a V_{L} comprising the amino acid sequences set forth as SEQ ID NOs: 481 and 485, respectively, and specifically binds to a coronavirus spike protein, and neutralizes a coronavirus. The coronavirus can be SARS-CoV-2.

In some embodiments, the disclosed antibodies inhibit viral entry and/or replication.

jjj. Additional Antibodies

In some examples, antibodies that bind to an epitope of interest can be identified based on their ability to cross-compete (for example, to competitively inhibit the binding of, in a statistically significant manner) with the antibodies provided herein in binding assays. In other examples, antibodies that bind to an epitope of interest can be identified based on their ability to cross-compete (for example, to competitively inhibit the binding of, in a statistically significant manner) with the one or more of the antibodies provided herein in binding assays.

Human antibodies that bind to the same epitope on the spike of the coronavirus protein, to which the disclosed antibodies bind can be produced using any suitable method. Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Additional human antibodies that bind to the same epitope can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain.

Antibodies and antigen binding fragments that specifically bind to the same epitope can also be isolated by screening combinatorial libraries for antibodies with the desired binding characteristics. For example, by generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Competitive binding assays, similar to those disclosed in the examples section below, can be used to select antibodies with the desired binding characteristics.

### 2. Additional Description of Antibodies and Antigen Binding Fragments

An antibody or antigen binding fragment of the antibodies disclosed herein can be a human antibody or fragment thereof. Chimeric antibodies are also provided. The antibody or antigen binding fragment can include any suitable framework region, such as (but not limited to) a human framework region from another source, or an optimized framework region. Alternatively, a heterologous framework region, such as, but not limited to a mouse or monkey framework region, can be included in the heavy or light chain of the antibodies.

The antibody can be of any isotype. The antibody can be, for example, an IgA, IgM or an IgG antibody, such as IgG₁, IgG₂, IgG₃, or IgG₄. The class of an antibody that specifically binds to a coronavirus spike protein can be switched with another. In one aspect, a nucleic acid molecule encoding V_{L} or V_{H} is isolated such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved, for example, using a vector or nucleic acid molecule that comprises a C_{L} or C_{H} chain. For example, an antibody that specifically binds the spike protein, that was originally IgG may be class switched to an IgA. Class switching can be used to convert one IgG subclass to another, such as from IgG₁ to IgG₂, IgG3, or IgG4.

In some examples, the disclosed antibodies are oligomers of antibodies, such as dimers, trimers, tetramers, pentamers, hexamers, septamers, octomers and so on.

The antibody or antigen binding fragment can be derivatized or linked to another molecule (such as another peptide or protein). In general, the antibody or antigen binding fragment is derivatized such that the binding to the spike protein is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

(a) Binding affinity

In several embodiments, the antibody or antigen binding fragment specifically binds the coronavirus spike protein with an affinity (e.g., measured by K_{D}) of no more than 1.0×10⁻⁸ M, no more than 5.0×10⁻⁸ M, no more than 1.0×10⁻⁹ M, no more than 5.0×10⁻⁹ M, no more than 1.0×10⁻¹⁰ M, no more than 5.0×10⁻¹⁰ M, or no more than 1.0×10⁻¹ M. K_{D} can be measured, for example, by a radiolabeled antigen binding assay (RIA) performed with the Fab version of an antibody of interest and its antigen. In one assay, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293(4):865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (NUNC™ Catalog #269620), 100 μM or 26 pM [¹²⁵I]-antigen are mixed with serial dilutions of a Fab of interest (e.g., consistent with assessment of the anti-VEGF antibody, Fab-12, in Presta et al., Cancer Res. 57(20):4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT™-20; PerkinElmer) is added, and the plates are counted on a TOPCOUNT™ gamma counter (PerkinElmer) for ten minutes. Concentrations of each Fab that give less than or equal to 20% of maximal binding are chosen for use in competitive binding assays.

In another assay, K_{D} can be measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE®, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. Affinity can also be measured by high throughput SPR using the Carterra LSA.

### (b) Bispecific Antibodies

In some embodiments, a multi-specific antibody, or a bi-specific antibody, such as a dual variable domain antibody (DVD-IG™) is provided that comprises an antibody or antigen binding fragment that specifically binds a coronavirus spike protein, as provided herein.

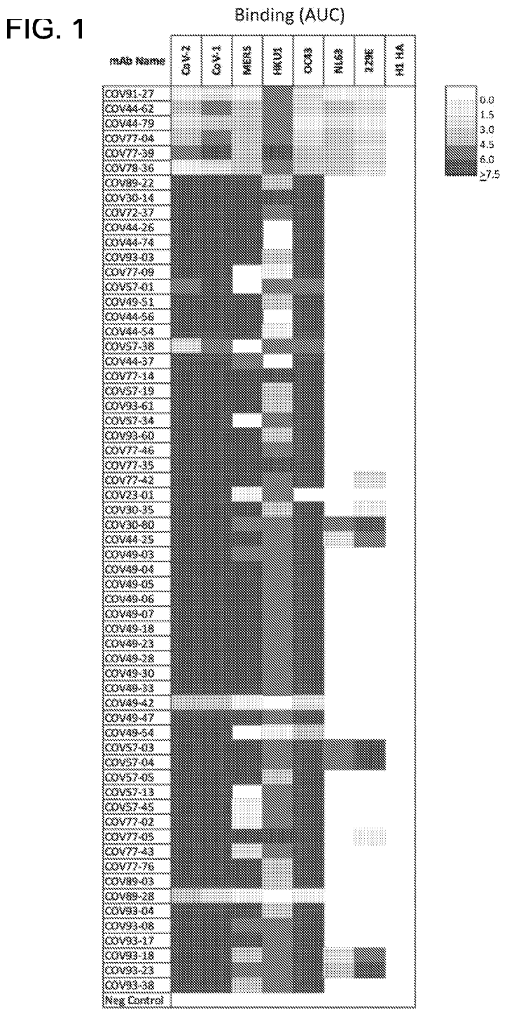

The bispecific tetravalent immunoglobulin known as the dual variable domain immunoglobulin or DVD-immunoglobulin molecule is disclosed in Wu et al., MAbs. 2009; 1:339-47, doi: 10.4161/mabs.1.4.8755, incorporated herein by reference. See also Nat Biotechnol. 2007 November; 25(11):1290-7. doi: 10.1038/nbt1345. Epub 2007 Oct. 14., also incorporated herein by reference. A DVD-immunoglobulin molecule includes two heavy chains and two light chains. Unlike IgG, however, both heavy and light chains of a DVD-immunoglobulin molecule contain an additional variable domain (VD) connected via a linker sequence at the N-termini of the VH and VL of an existing monoclonal antibody (mAb). Thus, when the heavy and the light chains combine, the resulting DVD-immunoglobulin molecule contains four antigen recognition sites, see Jakob et al., Mabs 5: 358-363, 2013, incorporated herein by reference, see FIG. 1 for schematic and space-filling diagrams. A DVD-immunoglobulin molecule functions to bind two different antigens on each DFab simultaneously.

The outermost or N-terminal variable domain is termed VD1 and the innermost variable domain is termed VD2; the VD2 is proximal to the C-terminal CH1 or CL. As disclosed in Jakob et al., supra, DVD-immunoglobulin molecules can be manufactured and purified to homogeneity in large quantities, have pharmacological properties similar to those of a conventional IgG₁, and show in vivo efficacy.

Any of the disclosed monoclonal antibodies can be included in a DVD-immunoglobulin format.

In other embodiments, any suitable method can be used to design and produce a bispecific antibody, such as crosslinking two or more antibodies, antigen binding fragments (such as scFvs) of the same type or of different types. Exemplary methods of making multispecific antibodies, such as bispecific antibodies, include those described in PCT Pub. No. WO2013/163427, which is incorporated by reference herein in its entirety. Non-limiting examples of suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate).

The multi-specific antibody may have any suitable format that allows for binding to the coronavirus spike protein by the antibody or antigen binding fragment as provided herein. Bispecific single chain antibodies can be encoded by a single nucleic acid molecule. Non-limiting examples of bispecific single chain antibodies, as well as methods of constructing such antibodies are provided in U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538. Additional examples of bispecific single chain antibodies can be found in PCT application No. WO 99/54440; Mack et al., J. Immunol., 158(8):3965-3970, 1997; Mack et al., Proc. Natl. Acad. Sci. U.S.A., 92(15):7021-7025, 1995; Kufer et al., Cancer Immunol. Immunother., 45(3-4):193-197, 1997; Löffler et al., Blood, 95(6):2098-2103, 2000; and Brühl et al., J. Immunol., 166(4):2420-2426, 2001. Production of bispecific Fab-scFv (“bibody”) molecules are described, for example, in Schoonjans et al. (J. Immunol., 165(12):7050-7057, 2000) and Willems et al. (J. Chromatogr. B Analyt. Technol. Biomed Life Sci. 786(1-2):161-176, 2003). For bibodies, a scFv molecule can be fused to one of the VL-CL (L) or VH-CH1 chains, e.g., to produce a bibody one scFv is fused to the C-term of a Fab chain.

### (c) Antigen Binding Fragments

Antigen binding fragments are encompassed by the present disclosure, such as Fab, F(ab′)₂, and Fv which include a heavy chain and V_{L} and specifically bind a coronavirus spike protein. These antibody fragments retain the ability to selectively bind with the antigen and are “antigen-binding” fragments. Non-limiting examples of such fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the V_{L} and V_{L} expressed as two chains; and
(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.
(6) A dimer of a single chain antibody (scFV₂), defined as a dimer of a scFV. This has also been termed a “miniantibody.”

Any suitable method of producing the above-discussed antigen binding fragments may be used. Non-limiting examples are provided in Harlow and Lane, Antibodies: A Laboratory Manual, 2^{nd}, Cold Spring Harbor Laboratory, New York, 2013.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

### (d) Variants

In some embodiments, amino acid sequence variants of the antibodies and bispecific antibodies provided herein are provided. For example, it may be desirable to improve the binding affinity and/or other biological properties of the antibody or bispecific antibody. Amino acid sequence variants of an antibody may be prepared by introducing appropriate modifications into the nucleotide sequence encoding the antibody V_{H} domain and/or V_{L} domain, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, variants having one or more amino acid substitutions are provided. Sites of interest for substitutional mutagenesis include the CDRs and the framework regions. Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions to increase yield.

In some embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 5.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 9. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 13.

In further embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 21.

In yet other embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 29. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 29.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 33. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 37.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 41. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 45.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 49. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 53.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 57. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 61.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 65. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 69.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 73. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 77.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 81. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 85.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 89. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 93.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 97. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 101.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 105. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 109.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 113. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 117.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 121. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 125.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 129. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 133.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 137. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 141.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 145. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 149.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 153. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 157.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 161. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 165.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 169. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 173.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 177. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 181.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 185. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 189.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 193. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 197.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 201. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 205.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 209. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 213.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 217. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 221.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 225. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 229.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 233. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 237.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 241. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 245.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 249. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 253.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 257. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 261.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 265. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 269.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 273. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 277.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 281. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 285.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 289. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 293.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 297. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 301.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 305. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 309.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 313. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 317.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 321. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 325.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 329. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 333.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 337. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 341.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 345. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 349.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 353. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 357.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 361. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 365.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 369. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 373.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 377. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 381.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 385. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 389.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 393. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 397.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 401. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 405.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 409. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 413.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 417. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 421.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 425. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 429.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 433. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 437.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 441. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 445.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 457. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 461.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 465. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 469.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 473. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 477.

In more embodiments, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 481. In some embodiments, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 485.

In some embodiments, the antibody or antigen binding fragment can include up to 10 (such as up to 1, up to 2, up to 3, up to 4, up to 5, up to 6, up to 7, up to 8, or up to 9) amino acid substitutions (such as conservative amino acid substitutions) in the framework regions of the heavy chain of the antibody/bispecific antibody, or the light chain of the antibody/bispecific antibody, or the heavy and light chains of the antibody/bispecific antibody, compared to known framework regions, or compared to the framework regions of the antibody, and maintain the specific binding activity for the epitope of the spike protein.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant V_{H} and V_{L} sequences provided above, each CDR either is unaltered, or contains no more than one, two or three amino acid substitutions. In some embodiments of the variant V_{H} and V_{L} sequences provided above, only the framework residues are modified so the CDRs are unchanged.

To increase binding affinity of the antibody, the V_{L} and V_{H} segments can be randomly mutated, such as within HCDR3 region or the LCDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for the spike protein. In particular examples, the V_{H} amino acid sequence is one of SEQ ID NOs:1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, 313, 321, 329, 337, 345, 353, 361, 369, 377, 385, 393, 401, 409, 417, 425, 433, 441, 449, 457, 465, 473, or 481. In other examples, the V_{L} amino acid sequence is one of SEQ ID NOs: 5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293, 301, 309, 317, 325, 333, 341, 349, 357, 365, 373, 381, 389, 397, 405, 413, 421, 429, 437,445, 453, 461, 469, 477, or 485, respectively.

In some embodiments, an antibody disclosed herein, an antigen binding fragment, or bispecific antibody is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15(1):26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336(5):1239-1249, 2004; Yamane-Ohnuki et al., Biotechnol. Bioeng. 87(5):614-622, 2004. Examples of cell lines capable of producing defucosylated antibodies include Lee 13 CHO cells deficient in protein fucosylation (Ripka et al., Arch. Biochem. Biophys. 249(2):533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87(5): 614-622, 2004; Kanda et al., Biotechnol. Bioeng., 94(4):680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In several embodiments, the constant region of the antibody or bispecific antibody comprises one or more amino acid substitutions to optimize in vivo half-life of the antibody. The serum half-life of IgG Abs is regulated by the neonatal Fc receptor (FcRn). Thus, in several embodiments, the antibody comprises an amino acid substitution that increases binding to the FcRn. Non-limiting examples of such substitutions include substitutions at IgG constant regions T250Q and M428L (see, e.g., Hinton et al., J Immunol., 176(1):346-356, 2006); M428L and N434S (the “LS” mutation, see, e.g., Zalevsky, et al., Nature Biotechnol., 28(2):157-159, 2010); N434A (see, e.g., Petkova et al., Int. Immunol., 18(12):1759-1769, 2006); T307A, E380A, and N434A (see, e.g., Petkova et al., Int. Immunol., 18(12):1759-1769, 2006); and M252Y, S254T, and T256E (see, e.g., Dall'Acqua et al., J. Biol. Chem., 281(33):23514-23524, 2006). The disclosed antibodies and antigen binding fragments can be linked to or comprise an Fc polypeptide including any of the substitutions listed above, for example, the Fc polypeptide can include the M428L and N434S substitutions.

In some embodiments, an antibody or bispecific antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### B. Conjugates

The antibodies, antigen binding fragments, and bispecific antibodies that specifically bind to a coronavirus spike protein, as disclosed herein, can be conjugated to an agent, such as an effector molecule or detectable marker. Both covalent and noncovalent attachment means may be used. Various effector molecules and detectable markers can be used, including (but not limited to) toxins and radioactive agents such as ¹²⁵I, ³²P, ¹⁴C, ³H and ³⁵S and other labels, target moieties, enzymes and ligands, etc. The choice of a particular effector molecule or detectable marker depends on the particular target molecule or cell, and the desired biological effect.

The procedure for attaching a detectable marker to an antibody, antigen binding fragment, or bispecific antibody. varies according to the chemical structure of the effector. Polypeptides typically contain a variety of functional groups, such as carboxyl (—COOH), free amine (—NH₂) or sulfhydryl (—SH) groups, which are available for reaction with a suitable functional group on a polypeptide to result in the binding of the effector molecule or detectable marker. Alternatively, the antibody, antigen binding fragment, or bispecific antibody, is derivatized to expose or attach additional reactive functional groups. The derivatization may involve attachment of any suitable linker molecule. The linker is capable of forming covalent bonds to both the antibody or antigen binding fragment and to the effector molecule or detectable marker. Suitable linkers include, but are not limited to, straight or branched-chain carbon linkers, heterocyclic carbon linkers, or peptide linkers. Where the antibody, antigen binding fragment, or bispecific antibody, and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody or antigen binding fragment or bispecific antibody can be determined.

The antibody, antigen binding fragment or bispecific antibody can be conjugated with a detectable marker; for example, a detectable marker capable of detection by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody, antigen binding fragment, or bispecific antibody, can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody, antigen binding fragment, or bispecific antibody, may also be conjugated with biotin, and detected through indirect measurement of avidin or streptavidin binding. It should be noted that the avidin itself can be conjugated with an enzyme or a fluorescent label.

The antibody, antigen binding fragment or bispecific antibody, can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody, antigen binding fragment, or bispecific antibody, may also be labeled with a predetermined polypeptide epitope recognized by a secondary reporter (such as leucine zipper pair sequences, binding sites for secondary antibodies, metal binding domains, epitope tags).

The antibody, antigen binding fragment or bispecific antibody, can also be conjugated with a radiolabeled amino acid, for example, for diagnostic purposes. For instance, the radiolabel may be used to detect a coronavirus by radiography, emission spectra, or other diagnostic techniques. Examples of labels for polypeptides include, but are not limited to, the following radioisotopes: ³H, ¹⁴C, ³⁵S, ⁹⁰Y, ^{99m}Tc, ¹¹¹In, ¹²⁵I, ¹³¹I. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of detectable marker moieties per antibody, antigen binding fragment, or bispecific antibody in a conjugate can range, for example, from 1 to 20 moieties per antibody or antigen binding fragment. In some embodiments, the average number of effector molecules or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

### C. Polynucleotides and Expression

Nucleic acid molecules (for example, cDNA or RNA molecules) encoding the amino acid sequences of antibodies, antigen binding fragments, bispecific antibodies, and conjugates that specifically bind to a coronavirus spike protein, as disclosed herein, are provided. Nucleic acids encoding these molecules can readily be produced using the amino acid sequences provided herein (such as the CDR sequences and V_{H} and V_{L} sequences), sequences available in the art (such as framework or constant region sequences), and the genetic code. In several embodiments, nucleic acid molecules can encode the V_{H}, the V_{L}, or both the V_{H} and V_{L} (for example in a bicistronic expression vector) of a disclosed antibody or antigen binding fragment. In some embodiments, the nucleic acid molecules encode an scFv. In several embodiments, the nucleic acid molecules can be expressed in a host cell (such as a mammalian cell) to produce a disclosed antibody or antigen binding fragment. Nucleic acid molecules encoding an scFv are provided.

The genetic code can be used to construct a variety of functionally equivalent nucleic acid sequences, such as nucleic acids which differ in their sequence but which encode the same antibody sequence, or encode a conjugate or fusion protein including the V_{L} and/or V_{H} nucleic acid sequence.

In a non-limiting example, an isolated nucleic acid molecule encodes the V_{H} of a disclosed antibody. In another non-limiting example, the nucleic acid molecule encodes the V_{L} of a disclosed antibody. In further non-limiting examples, the nucleic acid molecule can encode a bi-specific antibody, such as in DVD-immunoglobulin format.

Nucleic acid molecules encoding the antibodies, antigen binding fragments, bispecific antibodies, and conjugates that specifically bind to a coronavirus spike protein can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. The antibodies, antigen binding fragments, and conjugates can be expressed as individual proteins including the V_{H} and/or V_{L} (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011). An immunoadhesin can also be expressed. Thus, in some examples, nucleic acids encoding a V_{H} and V_{L}, and immunoadhesin are provided. The nucleic acid sequences can optionally encode a leader sequence.

To create a scFv the V_{H}- and V_{L}-encoding DNA fragments can be operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker (see, e.g., Bird et al., Science, 242(4877):423-426, 1988; Huston et al., Proc. Natl. Acad. Sci. U.S.A., 85(16):5879-5883, 1988; McCafferty et al., Nature, 348:552-554, 1990; Kontermann and Dubel (Eds.), Antibody Engineering, Vols. 1-2, 2^{nd} ed., Springer-Verlag, 2010; Greenfield (Ed.), Antibodies: A Laboratory Manual, 2^{nd} ed. New York: Cold Spring Harbor Laboratory Press, 2014). Optionally, a cleavage site can be included in a linker, such as a furin cleavage site.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to a coronavirus spike protein and another antigen. The encoded V_{H} and V_{L} optionally can include a furin cleavage site between the V_{H} and V_{L} domains. Linkers can also be encoded, such as when the nucleic acid molecule encodes a bi-specific antibody in DVD-IG™ format.

One or more DNA sequences encoding the antibodies, antigen binding fragments, bispecific antibodies, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and myeloma cell lines, can be used to express the disclosed antibodies and antigen binding fragments. Methods of stable transfer, meaning that the foreign DNA is continuously maintained in the host may be used. Hybridomas expressing the antibodies of interest are also encompassed by this disclosure.

The expression of nucleic acids encoding the antibodies, antigen binding fragments, and bispecific antibodies (such as DVD-immunoglobulin antibodies) described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lamda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, antigen binding fragments, bispecific antibodies, and conjugates can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The antibodies, antigen binding fragment, and conjugates need not be 100% pure. Once purified, partially or to homogeneity as desired, if to be used prophylatically, the polypeptides should be substantially free of endotoxin.

Methods for expression of antibodies, antigen binding fragments, bispecific antibodies, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### D. Methods and Compositions

### 1. Inhibiting a Coronavirus Infection

Methods are disclosed herein for the inhibition of a coronavirus infection in a subject. These methods include the administration on of an effective amount (that is, an amount effective to inhibit the infection in the subject) of a disclosed antibody, antigen binding fragment thereof, or bispecific antibody, or a nucleic acid encoding such an antibody, antigen binding fragment, or bispecific antibody to the subject.

In some embodiments, the disclosed monoclonal antibody binds the stem helix in the S2 domain of the Spike protein. These methods include an effective amount a disclosed antibody, antigen binding fragment, or bispecific antibody, or a nucleic acid encoding such an antibody, antigen binding fragment, or bispecific antibody, to the subject. The antibody can be, for example, COV30-14, COV44-26, COV44-37, COV44-54, COV44-56, COV44-74, COV49-51, COV72-37, COV77-09, COV93-03, or COV89-22. Combinations of these antibodies are also of use.

In some embodiments, the antibody or antigen binding fragment neutralizes a betacoronavirus and an alphacoronavirus. Thus, methods are disclosed herein for inhibiting a betacoronavirus infection and an alphacoronavirus infection in as subject. These methods include an effective amount of a disclosed antibody, antigen binding fragment thereof, or bispecific antibody, or a nucleic acid encoding such an antibody, antigen binding fragment, or bispecific antibody, to the subject. The antibody can be, for example, COV89-22, COV44-62, COV44-79, COV30-14, COV72-37, COV93-03, COV91-27, COV49-51, COV44-74, COV44-56, COV44-26, or COV44-54. Combinations of these antibodies are also of use.

In some embodiments, the antibody binds a spike protein from at least three betacoronaviruses selected from the group consisting of SARS-CoV-2, SARS-CoV, MERS-CoV, HKU1, and OC43. Thus, methods are disclosed for administering one or more of these coronavirus infections in a subject. These methods include an effective amount of a disclosed antibody, antigen binding fragment thereof, or bispecific antibody, or a nucleic acid encoding such an antibody, antigen binding fragment, or bispecific antibody, to the subject. The antibody can be, for example, COV30-14, COV44-26, COV44-37, COV44-54, COV44-56, COV44-74, COV49-51, COV72-37, COV77-09, COV93-03, COV89-22, COV23-01, COV30-35, COV30-80, COV44-25, COV49-03, COV49-04, COV49-05, COV49-06, COV49-07, COV49-18, COV49-23, COV49-28, COV49-30, COV49-33, COV49-42, COV49-47, COV49-54, COV57-01, COV57-03, COV57-04, COV57-05, COV57-13, COV57-19, COV57-34, COV57-38, COV57-45, COV77-02, COV77-05, COV77-14, COV77-35, COV77-42, COV77-43, COV77-46, COV77-76, COV93-04, COV93-08, COV93-17, COV93-18, COV93-23, COV93-38, COV93-60, COV93-61, COV89-03, or COV89-28. Combinations of these antibodies are also of use.

In further embodiments, the antibody or antigen binding fragment specifically binds a spike protein from SARS-CoV-2, SARS-CoV, MERS-CoV, HKU1, OC43, NL63 and 229E. Thus, methods are disclosed for inhibiting one or more of these coronavirus infections in a subject. These methods include an effective amount of a disclosed antibody, antigen binding fragment thereof, or bispecific antibody, or a nucleic acid encoding such an antibody, antigen binding fragment, or bispecific antibody, to the subject. The antibody can be, for example, COV91-27, COV44-62, COV44-79, COV77-04, COV77-39, or COV78-36. Combinations of these antibodies are also of use.

The disclosed methods can include administering to the subject an effective amount (that is, an amount effective to inhibit the infection in the subject) of a disclosed antibody, antigen binding fragment, or bispecific antibody, or a nucleic acid encoding such an antibody, antigen binding fragment, or bispecific antibody, to a subject at risk of a coronavirus infection or having the coronavirus infection. The methods can be used pre-exposure or post-exposure. In some embodiments, the antibody or antigen binding fragment can be used in the form of a bi-specific antibody, such as a DVD-IG™

The infection does not need to be completely eliminated or inhibited for the method to be effective. For example, the method can decrease the infection by a desired amount, for example by at least 10%, at least 20%, at least 50%, at least 60%, at least 70%, at least 80%, at least 90%, at least 95%, at least 98%, or even at least 100% (elimination or prevention of detectable coronavirus infection) as compared to the coronavirus infection in the absence of the treatment. In some embodiments, the subject can also be treated with an effective amount of an additional agent, such as an anti-viral agent.

In some embodiments, administration of an effective amount of a disclosed antibody, antigen binding fragment, bispecific antibody, or nucleic acid molecule, inhibits the establishment of an infection and/or subsequent disease progression in a subject, which can encompass any statistically significant reduction in activity (for example, growth or invasion) or symptoms of the coronavirus infection in the subject.

Methods are disclosed herein for the inhibition of a coronavirus replication in a subject. The methods include administering to the subject an effective amount (that is, an amount effective to inhibit replication in the subject) of a disclosed antibody, antigen binding fragment, bispecific antibody, or a nucleic acid encoding such an antibody, antigen binding fragment, or bispecific antibody, to a subject at risk of a coronavirus infection or having a coronavirus infection. The methods can be used pre-exposure or post-exposure.

Methods are disclosed for treating a coronavirus infection in a subject. Methods are also disclosed for preventing a coronavirus infection in a subject. These methods include administering one or more of the disclosed antibodies, antigen binding fragments, bispecific antibodies, or nucleic acid molecule encoding such molecules, or a composition including such molecules, as disclosed herein.

Antibodies, antigen binding fragments thereof, and bispecific antibodies can be administered by intravenous infusion. Doses of the antibody, antigen binding fragment, or bispecific antibody vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some embodiments, the dose of the antibody, antigen binding fragment or bispecific antibody can be from about 0.5 mg/kg to about 5 mg/kg, such as a dose of about 1 mg/kg, about 2 mg/kg, about 3 mg/kg, about 4 mg/kg or about 5 mg/kg. The antibody, antigen binding fragment, or bispecific antibody is administered according to a dosing schedule determined by a medical practitioner. In some examples, the antibody, antigen binding fragment or bispecific antibody is administered weekly, every two weeks, every three weeks or every four weeks.

In some embodiments, the method of inhibiting the infection in a subject further comprises administration of one or more additional agents to the subject. Additional agents of interest include, but are not limited to, anti-viral agents such as hydroxychloroquine, arbidol, remdesivir, favipiravir, baricitinib, lopinavir/ritonavir, Zinc ions, and interferon beta-1b, or their combinations.

In some embodiments, the method comprises administration of a first antibody that specifically binds to a coronavirus spike protein as disclosed herein and a second antibody that also specifically binds to a coronavirus protein, such as a different epitope of the coronavirus protein Thus, 2, 3, 4, 5, 6, 7, 8, 9, 10, 11, 12, 13, 14, 15, 16, 17, 18, 19 or 20 of the disclosed antibodies, or antigen binding fragments thereof, can be administered to the subject. The method can include administering at least 2, 3, 4, or 5 of the disclosed monoclonal antibodies or antigen binding fragments. Nucleic acid molecules are also of use in these embodiments. Combinations of these bispecific antibodies, such as one, two, three, four, or five of these bispecific antibodies can be administered to the subject.

In some embodiments, a subject is administered DNA or RNA encoding a disclosed antibody, antigen binding fragment, or bispecific antibody, to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, antigen binding fragments thereof, or bispecific antibody can be placed under the control of a promoter to increase expression. The methods include liposomal delivery of the nucleic acids. Such methods can be applied to the production of an antibody, or antigen binding fragments thereof. In some embodiments, a disclosed antibody or antigen binding fragment is expressed in a subject using the pVRC8400 vector (described in Barouch et al., J. Virol., 79(14), 8828-8834, 2005, which is incorporated by reference herein).

In several embodiments, a subject (such as a human subject at risk of a coronavirus infection or having a coronavirus infection) can be administered an effective amount of an AAV viral vector that comprises one or more nucleic acid molecules encoding a disclosed antibody, antigen binding fragment, or bispecific antibody. The AAV viral vector is designed for expression of the nucleic acid molecules encoding a disclosed antibody, antigen binding fragment, or bispecific antibody, and administration of the effective amount of the AAV viral vector to the subject leads to expression of an effective amount of the antibody, antigen binding fragment, or bispecific antibody in the subject. Non-limiting examples of AAV viral vectors that can be used to express a disclosed antibody, antigen binding fragment, or bispecific antibody in a subject include those provided in Johnson et al., Nat. Med., 15(8):901-906, 2009 and Gardner et al., Nature, 519(7541):87-91, 2015, each of which is incorporated by reference herein in its entirety.

In one embodiment, a nucleic acid encoding a disclosed antibody, antigen binding fragment, or bispecific antibody is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including a disclosed antibody, antigen binding fragment, or bispecific antibody, conjugate, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit a coronavirus infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The coronavirus spike protein-specific antibody, antigen binding fragment, or bispecific antibody or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In an embodiment, the antibody, antigen binding fragment, bispecific antibody or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, bispecific antibody, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the antibody, antigen binding fragment, conjugate, or nucleic acid molecule encoding such molecules, or a composition including such molecules, over a pre-determined period. The osmotic pump or mini-pump can be implanted subcutaneously, or near a target site.

### 2. Compositions

Compositions are provided that include one or more of the coronavirus spike protein-specific antibody, antigen binding fragment, bispecific antibody, conjugate, or nucleic acid molecule encoding such molecules, that are disclosed herein in a pharmaceutically acceptable carrier. In some embodiments, the composition comprises two, three, four or more antibodies, antigen binding fragments, or bispecific antibodies, that specifically bind a coronavirus spike protein. The compositions are useful, for example, for example, for the inhibition or detection of a coronavirus infection, such as, but not limited to, a SARS-CoV-2 infection. In some embodiments, the compositions are of use for inhibiting an alphacoronavirus or a betacoronavirus infection. In other embodiments, the compositions are of use for inhibiting a SARS-CoV-2, SARS-CoV, MERS-CoV, HKU1, or a OC43 infection. In further embodiments, the compositions are of use for inhibiting a SARS-CoV-2, SARS-CoV, MERS-CoV, HKU1, OC43, NL63 or a 229E infection.

The compositions can be prepared in unit dosage forms, such as in a kit, for administration to a subject. The amount and timing of administration are at the discretion of the administering physician to achieve the desired purposes. The antibody, antigen binding fragment, bispecific antibody, conjugate, or nucleic acid molecule encoding such molecules can be formulated for systemic or local administration. In one example, the, antigen binding fragment, bispecific antibody, conjugate, or nucleic acid molecule encoding such molecules, is formulated for parenteral administration, such as intravenous administration.

In some embodiments, the antibody, antigen binding fragment, bispecific antibody, or conjugate thereof, in the composition is at least 70% (such as at least 75%, at least 80%, at least 85%, at least 90%, at least 95%, at least 96%, at least 97%, at least 98% or at least 99%) pure. In some embodiments, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the antibody, antigen binding fragment, bispecific antibody, conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by any suitable technique. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration comprises about 0.01 to about 30 mg/kg of antibody, antigen binding fragment, bispecific antibody, or conjugate per subject per day (or the corresponding dose of a conjugate including the antibody or antigen binding fragment). Any suitable method may be used for preparing administrable compositions; non-limiting examples are provided in such publications as Remington: The Science and Practice of Pharmacy, 22^{nd} ed., London, UK: Pharmaceutical Press, 2013. In some embodiments, the composition can be a liquid formulation including one or more antibodies, antigen binding fragments, or bispecific antibodies, in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

Antibodies, an antigen binding fragment thereof, a bispecific antibody, or a nucleic acid encoding such molecules, can be provided in lyophilized form and rehydrated with sterile water before administration, although they are also provided in sterile solutions of known concentration. A solution including the antibody, antigen binding fragment, bispecific antibody, or a nucleic acid encoding such molecules, can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. Antibodies, antigen binding fragments, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the compositions disclosed herein. Any suitable polymer may be used, such as a degradable or nondegradable polymeric matrix designed for use in controlled drug delivery. Alternatively, hydroxyapatite has been used as a microcarrier for controlled release of proteins. In yet another aspect, liposomes are used for controlled release as well as drug targeting of the lipid-capsulated drug.

### 2. Methods of Detection and Diagnosis

Methods are also provided for the detection of the presence of a coronavirus spike protein in vitro or in vivo. In one example, the presence of a coronavirus spike protein is detected in a biological sample from a subject and can be used to identify a subject with an infection.

In embodiments, the method detects the presence of at least one coronavirus in the biological same. In some embodiments, the method detects the presence of an alphacoronavirus or a betacoronavirus in a biological sample. In other embodiments, the method detects the presence of SARS-CoV-2, SARS-CoV, MERS-CoV, HKU1, or a OC43 in a biological sample. In further embodiments, the method detects the presence of SARS-CoV-2, SARS-CoV, MERS-CoV, HKU1, OC43, NL63 or a 229E in a biological sample. In further embodiments, the methods detects the presence of at least SARS-CoV-2 in the biological sample.

The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, with an antibody, antigen binding fragment, or bispecific antibody, that specifically binds to a coronavirus spike protein, or conjugate thereof (e.g., a conjugate including a detectable marker) under conditions sufficient to form an immune complex, and detecting the immune complex (e.g., by detecting a detectable marker conjugated to the antibody or antigen binding fragment.

In one embodiment, the antibody, antigen binding fragment or bispecific antibody is directly labeled with a detectable marker. In another embodiment, the antibody (or antigen binding fragment or bispecific antibody) that binds the coronavirus spike protein (the primary antibody) is unlabeled and a secondary antibody or other molecule that can bind the primary antibody is utilized for detection. The secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially. Suitable labels for the antibody, antigen binding fragment, bispecific antibody or secondary antibody are known and described above, and include various enzymes, prosthetic groups, fluorescent materials, luminescent materials, magnetic agents and radioactive materials.

In some embodiments, the disclosed antibodies, antigen binding fragments thereof, or bispecific antibodies are used to test vaccines. For example, to test if a vaccine composition including a coronavirus spike protein or fragment thereof assumes a conformation including the epitope of a disclosed antibody. Thus, provided herein is a method for testing a vaccine, wherein the method comprises contacting a sample containing the vaccine, such as a coronavirus spike protein immunogen, with a disclosed antibody, antigen binding fragment, or bispecific antibody, under conditions sufficient for formation of an immune complex, and detecting the immune complex, to detect the vaccine including the epitope of interest in the sample. In one example, the detection of the immune complex in the sample indicates that vaccine component, such as the immunogen assumes a conformation capable of binding the antibody or antigen binding fragment.

### EXAMPLES

A panel of human monoclonal antibodies (mAbs) that target multiple coronaviruses were isolated. These mAbs can be used to inhibit a coronavirus infection, as prophylactics to prevent coronavirus infection, and as tools for the development of next-generation vaccines that protect more broadly against coronaviruses.

### Example 1

### Results

To evaluate the breadth of cross-reactivity of mAbs isolated from SARS-CoV-2 convalescent donors, antibodies were titrated against spike proteins of SARS-CoV-1, SARS-CoV-2 (Wuhan Hu-1), MERS-CoV, HCoV-HKU, HCoV-OC43, HCoV-NL63 and HCoV-229E using a multiplex bead-based assay (FIG. 1). This panel of spike proteins represents seven coronaviruses associated with human disease. Binding of the mAbs to the spike proteins was measured with the IntelliCyt IQUE® Screener flow cytometer and FACS data were analyzed with FlowJo (Version 10.8.1. Ashland, OR). mAb reactivity was analyzed by calculating area under the curve (AUC) for the IgG binding titration curves. All mAbs (n=61) showed cross-reactivity to both SARS-CoV-2 and SARS-CoV-1 spikes. All mAbs showed additional reactivity with at least one other non-SARS human coronavirus within the betacoronavirus genus. A subset of mAbs (n=6) showed particularly broad cross-reactivity across both the beta- and alphacoronavirus genera by binding to spike proteins of all seven human coronaviruses.

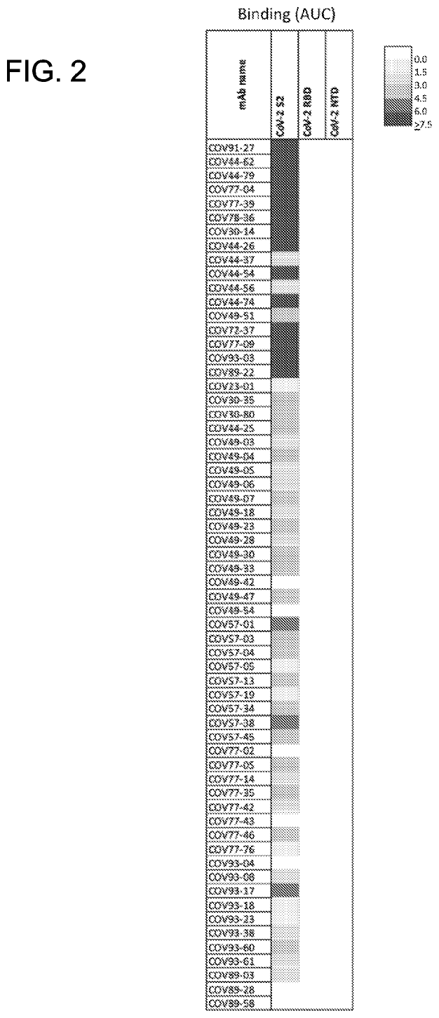

The SARS-CoV-2 spike glycoprotein is composed of a surface subunit (S1) and transmembrane subunit (S2). The S1 subunit is composed of four domains: a N-terminal domain (NTD), a C-terminal receptor-binding domain (RBD) and two subdomains (SD1 and SD2), while the S2 subunit is composed of N-terminal fusion peptide (FP), two heptad repeats (HR1 and HR2), a transmembrane domain (TM), and a cytoplasmic tail (CT). To determine which domains of the SARS-CoV-2 spike protein were targeted by the cross-reactive mAbs, mAb binding to the S2 subunit was assessed, as well as the receptor binding domain (RBD) and N-terminal domain (NTD) of the S1 subunit using a flow cytometry multiplex bead-based assay (FIG. 2). The majority of cross-reactive mAbs (n=54) were confirmed to have binding specificity to the SARS-CoV-2 S2 subunit, but not the RBD or NTD of the S1 subunit.

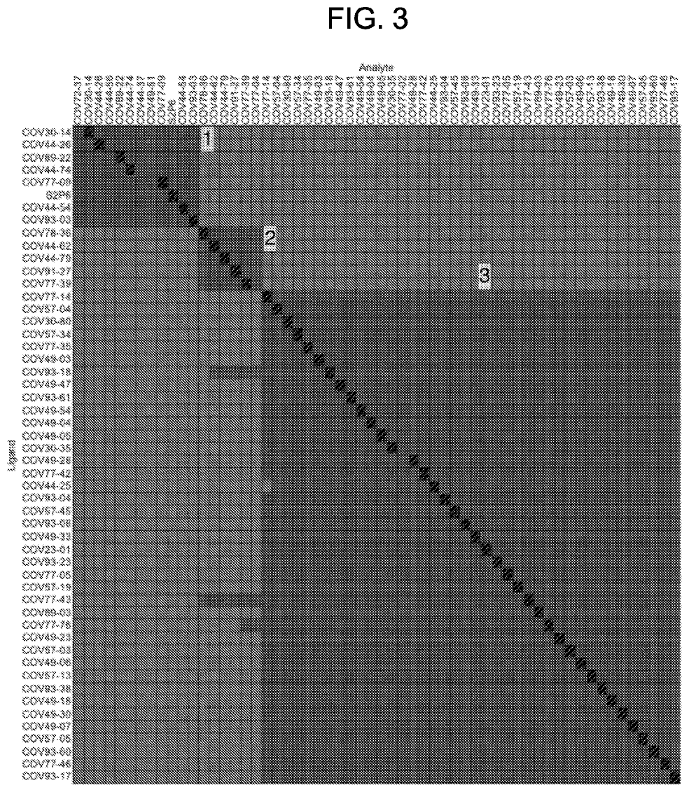

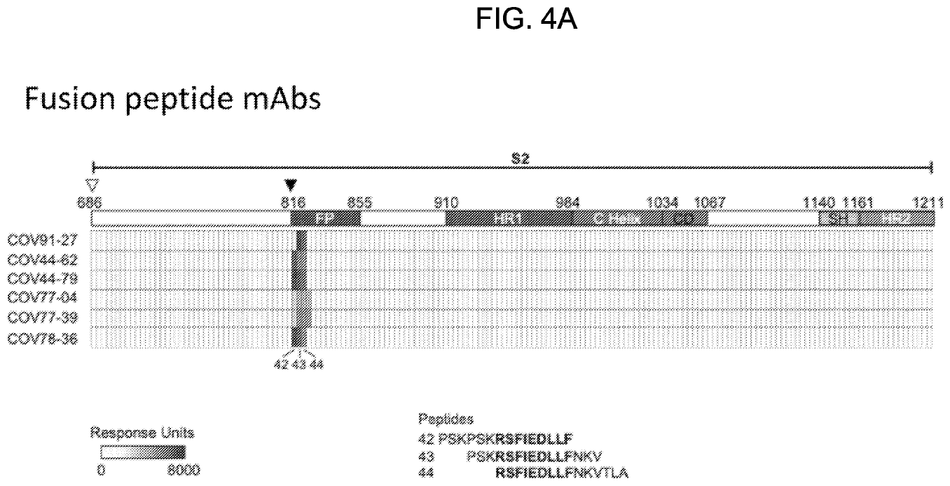

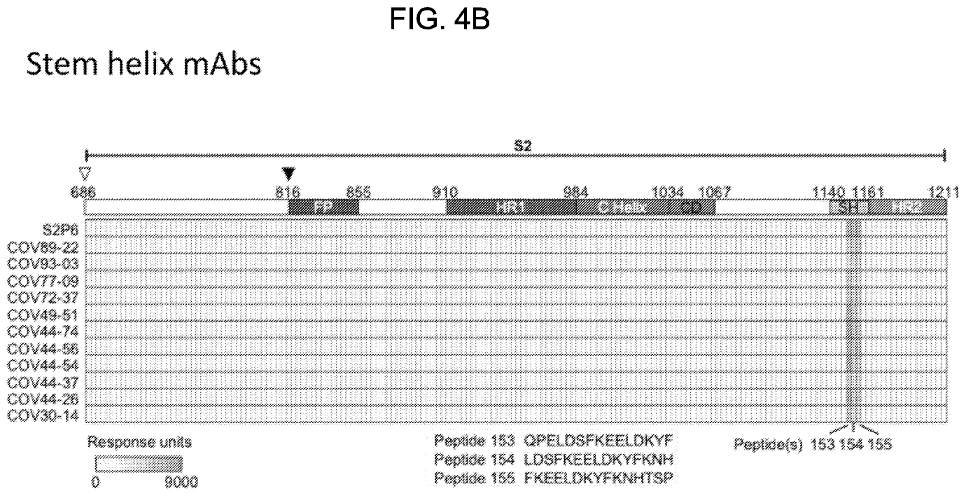

Subsequent SPR-based epitope binning analysis demonstrated that the mAbs could be separated into three distinct groups based on binding competition for the same epitope on the S2 subunit (FIG. 3). The mAbs sorted into Group 1 (n=11) competed for epitope binding with a previously described stem-helix targeting mAb S2P6 (Pinto, D., et al. 2021), while mAbs sorted into Group 2 (n=6) and Group 3 (n=40) bound to two distinct and separate epitopes on the S2 subunit. To further interrogate the specific site within the S2 subunit of the SARS CoV-2 spike protein that was targeted by the broadly reactive mAbs, all mAbs were subjected to surface plasmon resonance (SPR)-based measurements of binding to an array of peptides which spanned the length of the entire SARS-CoV-2 S2 subunit (Ser686¬Lys1211, Accession #YP_009724390.1) (FIGS. 4A-4B). Of the 54 mAbs confirmed by flow cytometry to bind to the S2 domain, 6 mAbs targeted overlapping peptides 42-44, which spanned the S2 subunit's fusion peptide (FP) region and share the ₈₁₅RSFIEDLLF₈₂₃ (SEQ ID NO: 498) motif (FIG. 4A). Eleven mAbs targeted overlapping peptides 153-155, which spanned the stem helix (SH) region of the S2 subunit (FIG. 4B). In separate, shotgun mutagenesis-based analyses, the remaining S2 subunit-binding mAbs were found to target a site N-terminal to the S2 cleavage site and fusion peptide region which we have termed K814⁺.

Efforts were focused on further characterizing the S2 fusion peptide and stem-helix specific antibodies. To determine the degree of conservation of the fusion peptide and stem helix sequences of coronaviruses, sequence alignment of SARS-CoV-2 spike proteins from a panel of diverse viral isolates was performed. It was found that each amino-acid position in the ₈₁₅RSFIEDLLF₈₂₃ (SEQ ID NO: 498) motif targeted by the fusion peptide mAbs was conserved in >90% of all viruses selected except at residue F817, which was conserved in <50% of isolates examined (FIG. 5A). For the stem helix, residues F1148, E1151, K1157 and N1158 were highly conserved (>90%) within the betacoronavirus subgenera but not among alphacoronaviruses (FIG. 5B), which is consistent with the betacoronavirus-restricted spike binding observed for the stem helix-specific mAbs. Notably, the fusion peptide and stem helix sequences are also identical among SARS-CoV-2 variants of concern that have been identified.

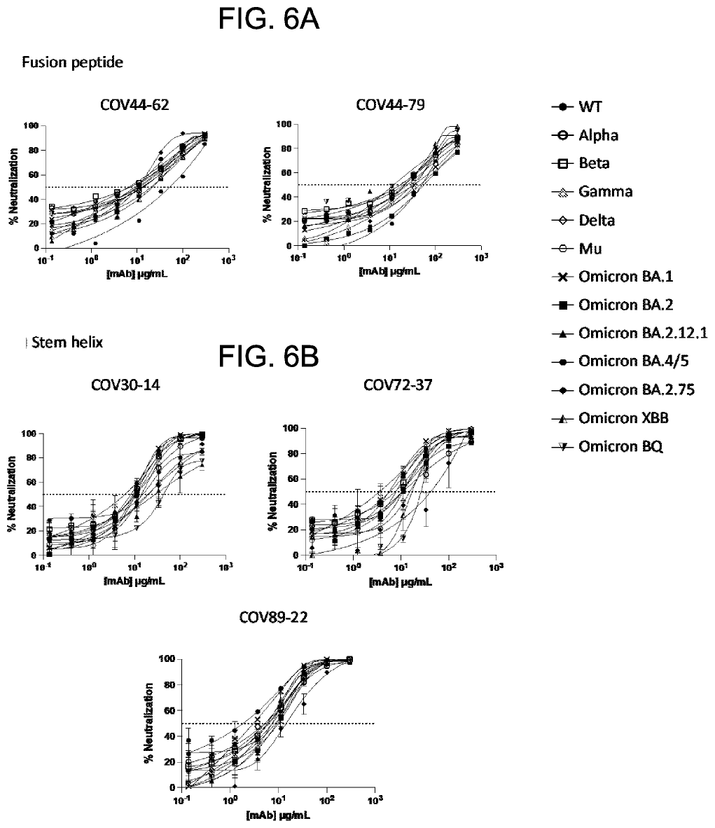

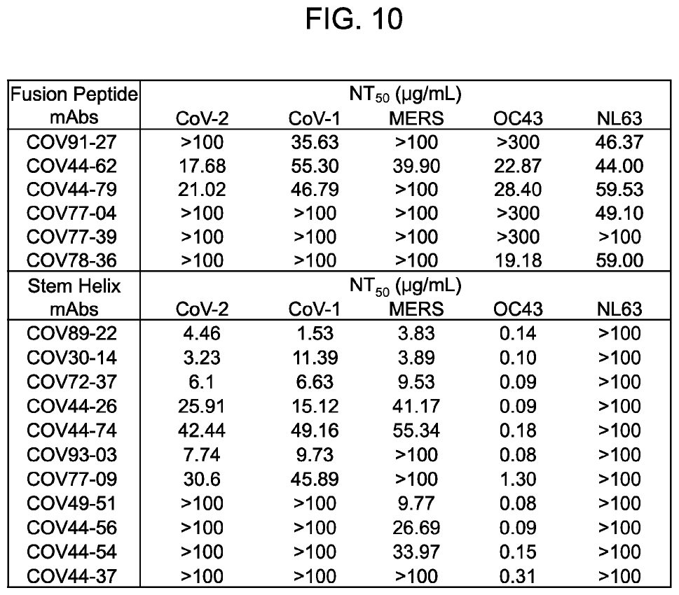

The neutralizing potency of each fusion peptide mAb (COV91-27, COV44-62, COV44-79, COV77-04, COV77-39 and COV78-36) and each stem helix mAb (COV89-22, COV30-14, COV72-37, COV44-26, COV44-74, COV93-03, COV77-09, COV49-51, COV44-56, COV44-54 and COV44-37) was analyzed against SARS-CoV-2 (Wuhan-Hu-1 isolate), SARS-CoV-1, MERS-CoV and HCoV-NL63 pseudovirus as well as authentic HCoV-OC43 (FIG. 10, which is Table 1). The antibodies showed different neutralization profiles, but consistent with the binding data, stem helix-targeting mAbs such as COV89-22 were able to neutralize multiple betacoronaviruses but not NL63, an alphacoronavirus. In contrast, fusion-peptide antibodies such as COV44-79 were able to neutralize both betacoronaviruses and NL63. The neutralizing potency of COV44-62, COV44-79, COV30-14, COV72-37 and COV89-22 were also analyzed against SARS-CoV-2 (WT) and SARS-CoV-2 variants of concern pseudoviruses. All five mAbs were able to neutralize the SARS-CoV-2 variants, albeit with different neutralization profiles depending on the variant tested (FIG. 6A-6B).

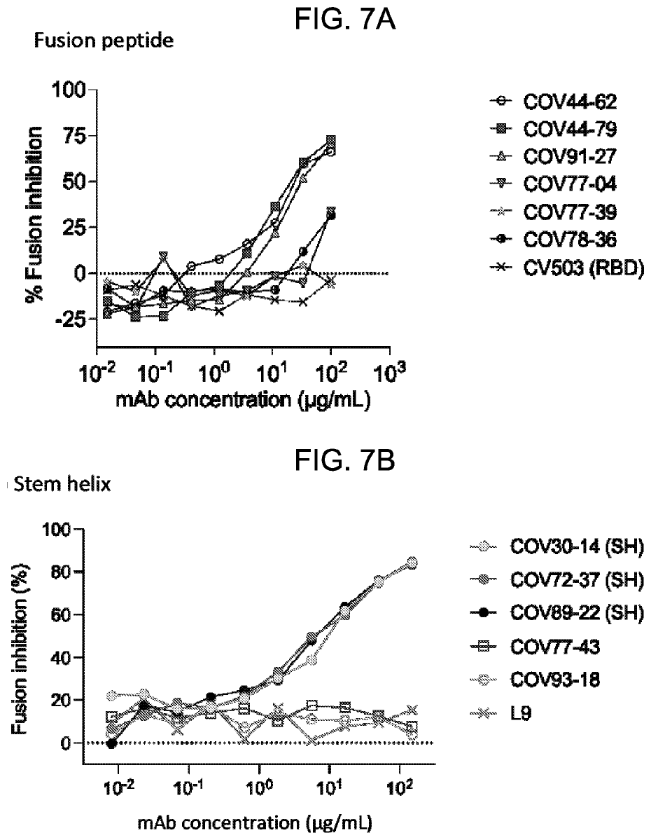

To interrogate potential mechanisms of mAb neutralization, the six fusion peptide-specific mAbs, as well as the stem helix specific mAbs COV89-22, COV30-14 and COV72-3, were assessed in a quantitative fusion inhibition assay. All of the stem helix mAbs tested as well as three of fusion peptide mAbs (COV44-62, COV44-79 and COV91-27) inhibited SARS-CoV-2 spike mediated fusion (FIG. 7A-7B).

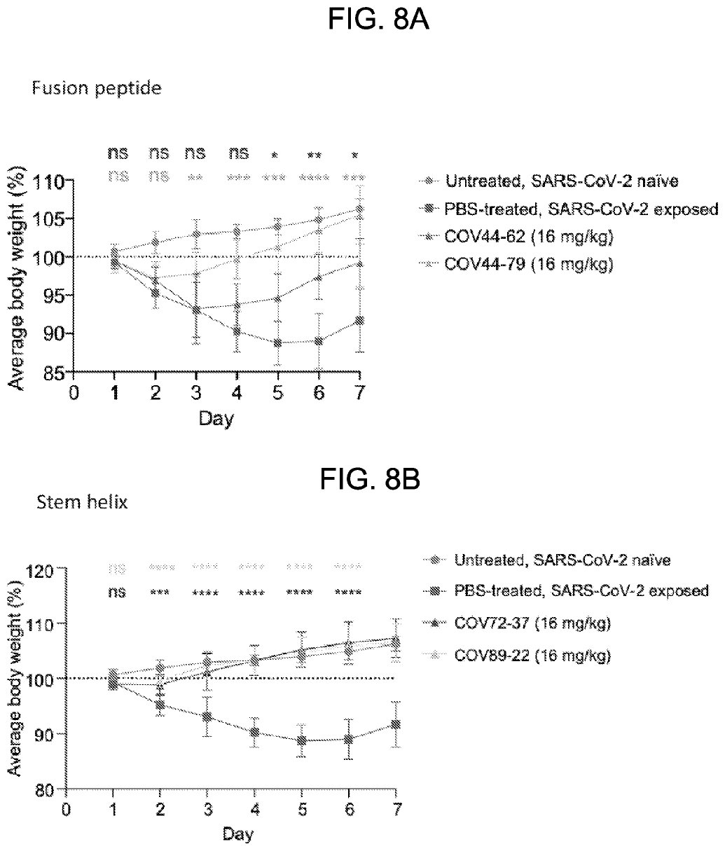

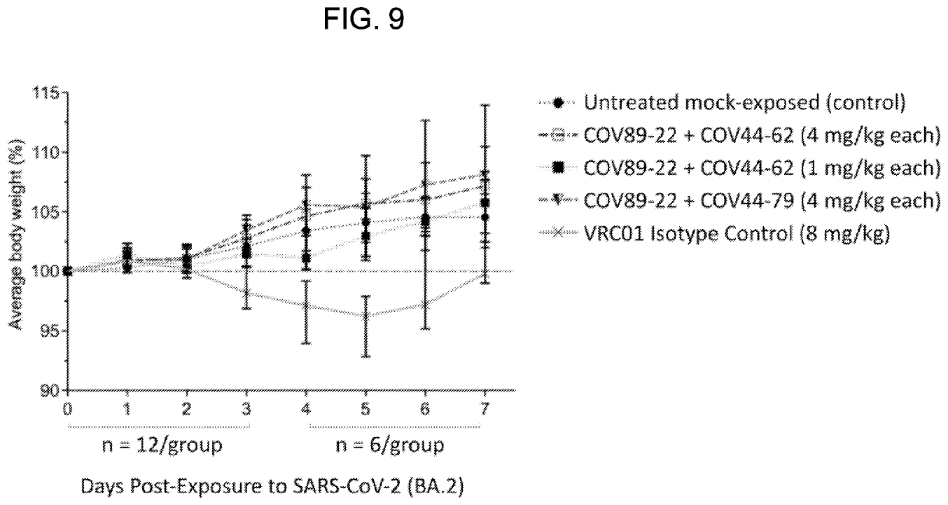

To determine the in vivo efficacy of the most potently neutralizing mAbs in our panel, COV44-62, COV44-79, COV72-37 and COV89-22 were evaluated against SARS-CoV-2 infection in Syrian hamsters, an animal model which recapitulates features of moderate to severe COVID-19 in humans. First, the Fc regions of each mAb were converted to hamster IgG2 to allow optimal Fc function. 16 mg/kg of each hamsterized mAb was then administered intraperitonially 24 hours prior to intranasal administration of 5 log₁₀ PFU of SARS-CoV-2 WA01. Untreated hamsters experienced around 10% weight loss through Day 6 post-infection. In contrast, hamsters treated with COV44-79, and to a lesser extent COV44-62, had a smaller decrease in body weight and recovered more quickly than untreated hamsters (FIG. 8A, P<0.01 from days 3-7 for COV44-79 and P<0.05 from days 5-7 for COV44-62). Notably, hamsters treated with COV89-22 and COV72-37 maintained body weight that was comparable to the uninfected controls throughout the study (FIG. 8B, P<0.001 relative to untreated hamsters from Days 2-7 for both mAbs). To determine whether delivery of fusion peptide and stem helix mAbs together would enhance the overall protective effects of the intervention, combinations of COV89-22+COV44-62 were evaluated at two doses (4 mg/kg each and 1 mg/kg each) as well as COV89-22+COV44-79 at 4 mg/kg each. Hamsters were infected with the SARS-CoV-2 BA.2. variant. Hamsters treated with the isotype control mAb VRC01 (Wu X., et al. 2010) experienced around 2.5-5% weight loss through Day 6 post-infection. In contrast, all hamsters that received mAb combinations maintained body weight similar to the mock-exposed control.

### Example 2

### Methods for Production of Antibodies

Step 1: Memory B Cell Isolation from PBMCs

Cryopreserved PBMCs from COVID-19 convalescent patients were thawed and stained with the following panel: DAPI (BD564907), CD14-BV510 (BioLegend 301842), CD3-BV510 (BioLegend 317332), CD56-BV510 (BioLegend 318340), CD19-ECD (Beckman Coulter IM2708U), CD21-BV711 (563163), IgA-Alexa Fluor 647 (Jackson Immunoresearch 109-606-011), IgD-PE-Cy7 (BD 561314), and IgM-PerCP-Cy5.5 (BD561285), CD27-ALEXA FLUOR®488 (BioLegend 393204) and CD38-APC-Cy7 (BioLegend 303534). The cells were sorted using the BD FACSARIA™ IIIu in a BSL3 facility and gated on live CD19⁺CD14⁻CD3⁻CD56⁻CD27⁺IgM⁻IgD⁻IgA⁺/IgA⁻ (memory B cells).

### Step 2: Optofluidic-Based Isolation of Individual Cross-Reactive Antibody Secreting B Cells

Sorted MBCs (CD19⁺ IgA⁺/IgG⁺) were resuspended with irr-3T3-CD40L feeder cells in IMDM supplemented with 10% HI-FBS, 100 ng/mL IL21, 0.5 μg/mL R848 and 1× MYCOZAP™. The cell suspension was seeded at a density of 50 MBCs and 3000 irr-3T3s per well of 384-well plates and co-cultured for 10 days to allow for MBC expansion and activation of Ig secretion. At day 9, culture supernatants were screened for IgA/IgG reactivity against beads coated with 10 μg/mL SARS-CoV-2, SARS-CoV-1, MERS-CoV, OC43-CoV, HKU1-CoV, 229E-CoV and NL63-CoV spike using the iQue screener. On day 10, MBCs from wells of interest were pooled, washed in MACS buffer (PBS supplemented with 0.5% w/v BSA and 2 mM EDTA) and approximately 23,000 cells were loaded onto an OptoSelect 11 k chip. OEP light cages were applied to sort single B cells into nanoliter-volume pens (nanopens) on the chip and antibody secreting cells were screened for cross-reactivity in a two-step assay. First, 7 μm streptavidin beads (Spherotech, SVP-60-5) coated in tandem with 10 μg/mL MERS-CoV spike and 10 μg/mL OC43-CoV spike were re-suspended in a cocktail of 2.5 μg/mL goat anti-human IgG-Alexa Fluor 647 (Jackson Immunoresearch 109-606-170) and goat anti-human IgA-Cγ3 (Jackson Immunoresearch 109-166-011), and immobilized in the channels of the OptoSelect 11 k chip. Binding of secreted antibody to the beads was detected in the CY5 or TRED channels by capturing images at 6 minutes intervals over a 30 minute time course. In the second step of the assay, the MERS/OC43 beads were replaced with 7 μm streptavidin beads coated with 10 μg/mL SARS-CoV-2 spike, and antibody binding was detected as before. OEP light cages were applied to export Individual cross-reactive mAb secreting B cells directly into DYNABEADS™ mRNA DIRECT™ lysis buffer (Life Technologies, 61011) in 96-well plates. Plates were sealed with Microseal foil film and immediately frozen on dry ice before transferring to −80° C. for long-term storage.

### Step 3: mAb Sequence Analysis and Expression

Heavy and light chain sequences were amplified from single B cell lysates using RT-PCR (Cho et al., Sci Transl Med 2021, 13:eabj5413; Wang et al., Immunity 2020, 53:733-744 e738: Tiller et al., J Immunol Methods 2008, 329:112-124) and resolved by Sanger Sequencing (Eurofins and Genewiz). Analyses of the VH and Vλ/Vκ genes, CDR3 sequences, and percentage of somatic mutations were carried out using Geneious Prime (Version 2021.0.3, geneious.com) and the International Immunogenetics Information System database (IMGT, imgt.org/) (Lefranc, Front Immunol 2014, 5:22). Matched pairs of antibody V_{H} and Vλ/Vκ sequences were commercially cloned into plasmids containing an IgG1 or relevant light chain backbone and expressed as recombinant antibody using CHO or 293 cells (Genscript). mAbs were also expressed in-house by transient transfection of Expi293 cells (Gibco, A14527) using the ExpiFectamine 293 Transfection Kit (Gibco, A14524) according to manufacturer's instructions. Recombinant IgG mAbs were purified using HiTrap Protein A columns (GE Healthcare Life Sciences).

### Example 3

### Description of Assays

### 1. Generation of Multiplexed CoV Antigen Beads

Streptavidin beads pre-labelled with individual intensities of PE-channel fluorophore (Spherotech SVFA-2558-6K and SVFB-2558-6K) or FITC-channel fluorophore (SVFA-2552-6K and SVFA-2552-6K) were incubated with 10 μg/mL each of the following biotinylated antigens: recombinant SARS-CoV-2 spike, SARS-CoV-2 RBD, SARS-CoV-2 NTD, SARS-CoV-1 spike and SARS-CoV-1 RBD (expressed as previously described (Cho et al., supra, 2021); MERS-CoV spike, HCoV-OC43 spike and H1N1 HA; HC0V-NL63-spike (Sino Biological 40604-V08B,) HCoV-229E spike (Sino Biological 40605-V08B), HCoV-HKU1 spike (Sino Biological 40606-V08B), and CD4. Antigens acquired in the His-tagged, un-biotinylated form were incubated with 2 μg/mL anti-His biotin (Invitrogen, MA1-21315-BTIN) for 20 min at room temperature before being used to label the streptavidin beads. After incubation with the antigens, beads were washed with 0.05% BSA w/v in PBS and incubated with 10 μg/mL of CD4 to block excess streptavidin sites. The blocked beads were washed twice with 0.05% BSA w/v in PBS and mixed to generate multiplexed configurations as required.

### 2. Recombinant mAb Binding to Coronavirus Antigens

Recombinant mAbs were diluted 4-fold in 0.05% BSA w/v in PBS to generate a 47.7 pg/mL-200 μg/mL dilution series. Multiplexed antigen-labelled beads were incubated with mAb titrations for 30 min at room temperature, then washed and stained with 2.5 μg/mL goat anti-human IgG Alexa Fluor 647 (Jackson Immunoresearch, 109-606-170). Samples were acquired on the IQUE® Screener Plus and FACS data were analyzed with FlowJo. Titration curves and AUC analyses were performed with GraphPad Prism and reported after correction using the AUC of the negative control CD4 population.

### 3. SARS-CoV-2 S2 Peptide Mapping

Peptides spanning the entire SARS-CoV-2 S2 domain (Ser686 Lys1211, Accession #YP_009724390.1) were commercially synthesized (JPT Peptide Technologies). Each peptide was 15 amino acids in length, overlapped its flanking peptides by 12 residues and carried an N-terminal biotin tag. A further 8 irrelevant oligomers representing random H1N1 haemagglutinin peptides were included in the panel as negative controls. The lyophilized biotinylated peptides were reconstituted to 1 mg/mL in DMSO, then further diluted in HBSTE supplemented with 0.05% BSA and directly coupled to the streptavidin surface of a SAD200M chip (Carterra) at 0.1 μg/mL and 10 μg/mL. Cross-reactive mAbs were successively injected onto the peptide array at 10 μg/mL and binding data were acquired over a 5 min association phase and 1 min dissociation phase using the Carterra LSA. 3 successive injections of 10 mM glycine pH 2.0 were used to regenerate antibody binding sites on the arrayed peptides after each antibody injection. Data were analyzed using the Epitope Software (Carterra).

### 4. Pseudovirus Neutralization Assays

Lentiviral based pseudo-viruses were produced similar to previously reported (Rogers et al., Science 2020 369:956-963). HEK293T cells were seeded in 6 well plates and grown to ˜80% confluency in DMEM (Lonza, 12-614F) with P/S, glutamine and 10% heat-inactivated FBS. 2.5 μg 2nd generation lentivirus backbone plasmid pCMV-dR8.2 dvpr (Addgene #8455), 2 μg pBOBI-FLuc (Addgene #170674) and 1 μg truncated coronavirus spike expressing plasmids (SARS: Addgene #170447; SARS2 #170442; MERS #170448; NL63 #172666; alpha strain #170451; beta #170449; gamma #170450; delta #172320; omicron #180375) were co-transfected in HEK293T with Lipofectamine 2000 (ThermoFisher Scientific cat. #11668019) to produce single-round infection-competent pseudoviruses. The amount was scaled up to 10 cm dish. The medium was changed 12-16 hours post transfection. Pseudovirus-containing supernatants were collected 48 and 72 hours post transfection, centrifuged at 1,500 g for 10 min and the viral titers were measured by luciferase activity in relative light units (RLU) (BRIGHT-GLO™ Luciferase Assay System, Promega cat. #E2620). The supernatants were aliquoted and stored at −80° C. until further use.

Pseudotyped viral neutralization assay was performed similar to previously reported (Rogers et al., Science 2020 369:956-963). 20 μL pseudovirus supernatant were added into 20 μL serial dilutions of purified antibodies (starting from 100 μg/mL and dilute by 3 fold) in 384-well plates (Corning, #3570). The mixture was incubated for one hour at 37° C., after which 5,000 HeLa-hACE2 cells/well (in 20 μL medium containing 30 μg/ml Dextran) were directly added to the mixture. After incubation at 37° C. for 42-48 h, the medium was aspirated and luciferase activity was measured by adding 25 μL 1× luciferase substrate. Neutralizing activity was calculated by reduction in luciferase activity compared to the virus controls. Fifty percent maximal inhibitory concentrations (IC50) were calculated using the dose-response-inhibition model with 4-parameter Hill slope equation in GraphPad Prism 9 (GraphPad Software).

### 5. Authentic HCoV-OC43-GFP Neutralization Assay

mAbs were serially diluted, mixed with OC43-GFP virus were incubated for 1 h at 37° C. Rhabdomyosarcoma (RD) cells were then inoculated with the mAb-virus mixture at a final MOI of 0.01. RD cells were subsequently incubated for 24 h at 37° C. Untreated uninfected cells were plated as negative controls, and untreated infected cells plated as positive controls (MinGFP and MaxGFP, respectively). Cells were harvested 24 h post-infection and GFP expression was measured by flow cytometry using the iQue Screener Plus (Intellicyt). The resulting data were analysed with FlowJo (Version 10.8.1) and % neutralization was calculated as 100×(1−(GFP−MinGFP)/(MaxGFP−MinGFP)).

### 6. Quantitative Fusion Inhibition Assay

Fusion inhibition was quantitatively measured using an in vitro assay. Donor cells (HEK-293s engineered to stably express hMyD88, Invivogen, 293-hmyd) were transfected with either SARS-CoV-2 spike or a control vector. The transfected donor cells were subsequently incubated with 3-fold serial dilutions of each mAb, then co-cultured with acceptor cells (HEK-293 cells engineered to stably express TMPRSS-2 and hACE2, as well as secreted embryonic alkaline phosphatase under control of the NF-kb promoter, Invivogen, hkb-hace2tpsa). Activation of secreted embryonic alkaline phosphatase (SEAP) occurs downstream of fusion between the donor cell-expressed spike protein and the acceptor cell-expressed hACE2 receptor. In this way, fusion inhibition was quantified by measuring SEAP activity levels in co-culture supernatants. Co-culture supernatants were mixed with Quantiblue substrate, and the chromogenic compound produced upon cleavage of the substrate by SEAP was measured using a spectrophotometer. Absorbance measurements were background corrected by subtracting the absorbance of donor cells transfected with the vector control (N). % Inhibition was determined using the formula (1−(E/N)/(P−N)); E—Abs635 of test mAb and P—Abs635 of 0 μg/mL mAb.

### 7. Coronavirus Spike Protein Sequence Conservation Analyses

[THERE ARE 11 ACCESSION NUMBERES FOR WHICH THE DATABASE IS NOT INDICATED. EACH IS HIGHLIGHED IN YELLOW BELOW PLEASE PROVIDE-IT IS LIKELY UNIPROT OR GENBANK®.] To evaluate the conservation of the primary protein structure of spike, a multiple sequence alignments were performed using the following full-length sequences: HCoV-229E (accession #NP_073551), HCoV-NL63 (accession #YP_003767), CCoV-HuPn-2018 (accession #QVL91811), Mouse Hepatitis Virus (Uniprot accession #P11224), HCoV-OC43 (Uniprot accession #P36334), HCoV-HKU1 N5 (Uniprot accession #Q0ZME7), BatCoV-HKU3 (Uniprot accession #Q3LZX1), BatCoV-RaTG13 (GENBANK® accession #QHR63300), BatCoV-Rs4231 (GENBANK® accession #AT098157), BatCoV-Rs3367 (GENBANK® accession #AGZ48818), BatCoV-WIV1 (GENBANK® accession #AGZ48831), Civet-SARS-CoV-007/004 (GENBANK® accession #AAU04646), Pangolin-CoV-GX-P2V (GENBANK® accession #QIQ54048), SARS-CoV-Tor2 (GENBANK® accession #AAP41037), SARS-CoV-Urbani (GENBANK® accession #AAP13441), SARS-CoV-2 Wuhan-Hu-1 (GENBANK® accession #YP_009724390), B.1.1.7 (GENBANK® accession #QWE88920), SARS-CoV-2 BA.2.12.1 (accession #UMZ92892), B.1.351 (GENBANK® accession #QRN78347), P.1 (GENBANK® accession #QVE55289), B.1.617.2 (GENBANK® accession #QWK65230), BA.1 (GENBANK® accession #UFO69279), BA.2 (GENBANK® accession #UJE45220), BA.2.75 (GENBANK® accession #UTM82166.1), BA.5 (GENBANK® accession #UOZ45804.1), Bat-CoV-HKU4 (Uniprot accession #A3EX94), BatCoV-HKU5 (GENBANK® accession #YP_001039962.1), MERS-EMC/2012 (GENBANK® accession #YP_009047204), BatCoV-GCCDC1 (Genbank accession #YP_009273005.1), BtRt-BetaCoV/GX2018 (GENBANK® accession #QJX58383.1), BatCoV-HKU9 (GENBANK® accession #ABN10911), Bat Hp-betacoronavirus/Zhejiang2013 (GENBANK® accession #YP_009072440), Avian Infectious Bronchitis Virus (accession #F4MIW6), TCoV (accession #B3FHU5), WhaleCoV-SW1 (accession #B2BW33), BuCoV-HKU11 (accession #ACJ12044), MuCoV-HKU13-3514 (accession #B6VDY7), PDCoV/Haiti/Human/0081-4/2014 (accession #QWE80492), PDCoV/Haiti/Human/0329-4/2015 (accession #QWE80508), ThCoV-HKU12 (accession #B6VDX8) and WiCoV-HKU20 (accession #H9BR25). Sequences were aligned using the MAFFT v7 server using a BLOSUM62 scoring matrix and L-INS-I algorithm.

### 8. Syrian Hamster Efficacy Studies

Hamster studies were carried out at the Integrated Research Facility, Division of Clinical Research (NIAID/NIH). Golden Syrian Hamsters were weighed prior to the start of the study. Equal numbers of males and females (5-6 weeks old) were then distributed into eight groups of n=12 according to weight and sex. For individual mAb studies, animals received 16 mg/kg of either COV44-62, COV44-79, COV72-37 or COV89-22 by the intra-peritoneal (IP) route 24 h prior to intranasal (IN) inoculation with either 5 log 10 pfu SARS-CoV-2 (WA01) or PBS for mock-exposed animals. For mAb cocktail studies, animals received combinations of 4 mg/kg each of COV44-62 and COV89-22 (total 8 mg/kg mAb), 1 mg/kg each of COV44-62 and COV89-22 (total 2 mg/kg mAb), 4 mg/kg each of COV44-79 and COV89-22 (total 8 mg/kg mAb) or VRC01 (total 8 mg/kg) by the intra-peritoneal (IP) route 24 h prior to intranasal (IN) inoculation with either 5 log 10 pfu SARS-CoV-2 (BA.2) or PBS for mock-exposed animals. For both studies, animals were weighed and observed daily for clinical signs of disease. On Day 3, half of the animals in each group were sacrificed. Remaining animals were sacrificed on Day 7. None of the animals used in the study reached endpoint criteria that would have required an unscheduled euthanasia. All hamster studies were blinded and animals were randomly assigned to their respective treatment groups.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. An isolated monoclonal antibody or antigen binding fragment thereof, comprising a heavy chain variable (V_{H}) region and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as any one of:
a) SEQ ID NOs: 17 and 21, respectively (COV44-79);
b) SEQ ID NOs: 9 and 13, respectively (COV44-62);
c) SEQ ID NOs: 1 and 5, respectively (COV89-22);
d) SEQ ID NOs: 25 and 29, respectively (COV30-14);
e) SEQ ID NOs: 33 and 37, respectively (COV72-37);
f) SEQ ID NOs: 49 and 53, respectively (COV91-27);
g) SEQ ID NOs: 41 and 45, respectively (COV93-03);
h) SEQ ID NOs: 57 and 61, respectively (COV49-51);
i) SEQ ID NOs: 65 and 69, respectively (COV44-74);
j) SEQ ID NOs: 73 and 77, respectively (COV44-56);
k) SEQ ID NOs: 81 and 85, respectively (COV44-26);
l) SEQ ID NOs: 89 and 93, respectively (COV44-54);
m) SEQ ID NOs: 97 and 101, respectively (COV23-01);
n) SEQ ID NOs: 105 and 109, respectively (COV49-03);
o) SEQ ID NOs: 113 and 117, respectively (COV49-04);
p) SEQ ID NOs: 121 and 125, respectively (COV49-05);
q) SEQ ID NOs: 129 and 133, respectively (COV49-06);
r) SEQ ID NOs: 137 and 141, respectively (COV49-07);
s) SEQ ID NOs: 145 and 149, respectively (COV49-18);
t) SEQ ID NOs: 153 and 157, respectively (COV49-23);
u) SEQ ID NOs: 161 and 165, respectively (COV49-28);
v) SEQ ID NOs: 169 and 173, respectively (COV49-30);
w) SEQ ID NOs: 177 and 181, respectively (COV49-33);
x) SEQ ID NOs: 185 and 189, respectively (COV49-42);
y) SEQ ID NOs: 193 and 197, respectively (COV49-47);
z) SEQ ID NOs: 201 and 205, respectively (COV49-54);
aa) SEQ ID NOs: 209 and 213, respectively (COV57-01);
bb) SEQ ID NOs: 217 and 221, respectively (COV57-03);
cc) SEQ ID NOs: 225 and 229, respectively (COV57-04);
dd) SEQ ID NOs: 233 and 237, respectively (COV57-05);
ee) SEQ ID NOs: 241 and 245, respectively (COV57-13);
ff) SEQ ID NOs: 249 and 253, respectively (COV57-19);
gg) SEQ ID NOs: 257 and 261, respectively (COV57-34);
hh) SEQ ID NOs: 265 and 269, respectively (COV57-38);
ii) SEQ ID NOs: 273 and 277, respectively (COV57-45);
jj) SEQ ID NOs: 281 and 285, respectively (COV77-02);
kk) SEQ ID NOs: 289 and 293, respectively (COV77-04);
ll) SEQ ID NOs: 297 and 301, respectively (COV77-05);
mm) SEQ ID NOs: 305 and 309, respectively (COV77-09);
nn) SEQ ID NOs: 313 and 317, respectively (COV77-14);
oo) SEQ ID NOs: 321 and 325, respectively (COV77-35);
pp) SEQ ID NOs: 329 and 333, respectively (COV77-39);
qq) SEQ ID NOs: 337 and 341, respectively (COV77-42);
rr) SEQ ID NOs: 345 and 349, respectively (COV77-43);
ss) SEQ ID NOs: 353 and 357, respectively (COV77-46);
tt) SEQ ID NOs: 361 and 365, respectively (COV77-76);
uu) SEQ ID NOs: 369 and 373, respectively (COV93-04);
vv) SEQ ID NOs: 377 and 381, respectively (COV93-08);
ww) SEQ ID NOs: 385 and 389, respectively (COV93-17);
xx) SEQ ID NOs: 393 and 397, respectively (COV93-18);
yy) SEQ ID NOs: 401 and 405, respectively (COV93-23);
zz) SEQ ID NOs: 409 and 413, respectively (COV78-36);
aaa) SEQ ID NOs: 417 and 421, respectively (COV93-38);
bbb) SEQ ID NOs: 425 and 429, respectively (COV93-60);
ccc) SEQ ID NOs: 433 and 437, respectively (COV93-61);
ddd) SEQ ID NOs: 441 and 445, respectively (COV89-03);
eee) SEQ ID NOs: 449 and 453, respectively (COV89-28);
fff) SEQ ID NOs: 457 and 461, respectively (COV30-35);
ggg) SEQ ID NOs: 465 and 469, respectively (COV30-80);
hhh) SEQ ID NOs: 473 and 477, respectively (COV44-25); or
iii) SEQ ID NOs: 481 and 485, respectively (COV44-37);
and wherein the monoclonal antibody specifically binds to a coronavirus spike protein.

2. The antibody or antigen binding fragment of claim 1, wherein the HCDR1, the HCDR2, the HCDR3, the LCDR1, the LCDR2, and the LCDR3 respectively comprise the amino acids sequences set forth as
a) SEQ ID NOs: 18, 19, 20, 22, 23 (AAS), and 24;
b) SEQ ID NOs: 10, 11, 12, 14, 15 (EVT), and 16;
c) SEQ ID NOs: 2, 3, 4, 6, 7 (DAS), and 8;
d) SEQ ID NOs: 26, 27, 28, 30, 31 (GES), and 32;
e) SEQ ID NOs: 34, 35, 36, 38, 39 (GAS), and 40;
f) SEQ ID NOs: 50, 51, 52, 54, 55 (DAS), and 56;
g) SEQ ID NOs: 42, 43, 44, 46, 47 (GAS), and 48;
h) SEQ ID NOs: 58, 59, 60, 62, 63 (GAS), and 64;
i) SEQ ID NOs: 66, 67, 68, 70, 71 (AAS), and 72;
j) SEQ ID NOs: 74, 75, 76, 78, 79 (LGS), and 80;
k) SEQ ID NOs: 82, 83, 84, 86, 87 (AAS), and 88;
l) SEQ ID NOs: 90, 91, 92, 94, 95 (AAS), and 96;
m) SEQ ID NOs: 98, 99, 100, 102, 103 (DAS), and 104;
n) SEQ ID NOs: 106, 107, 108, 110, 111 (KVS), and 112;
o) SEQ ID NOs: 114, 115, 116, 118, 119 (RVS), and 120;
p) SEQ ID NOs: 122, 123, 124, 126, 127 (QVS), and 128;
q) SEQ ID NOs: 130, 131, 132, 134, 135 (KVS), and 136;
r) SEQ ID NOs: 138, 139, 140, 142, 143 (KVS), and 144;
s) SEQ ID NOs: 146, 147, 148, 150, 151 (KVS), and 152;
t) SEQ ID NOs: 154, 155, 156, 158, 159 (QVS), and 160;
u) SEQ ID NOs: 162, 163, 164, 166, 167 (QVS), and 168;
v) SEQ ID NOs: 170, 171, 172, 174, 175 (KVS), and 176;
w) SEQ ID NOs: 178, 179, 180, 182, 183 (QVS), and 184;
x) SEQ ID NOs: 186, 187, 188, 190, 191 (AAS), and 192;
y) SEQ ID NOs: 194, 195, 196, 198, 199 (QVS), and 200;
z) SEQ ID NOs: 202, 203, 204, 206, 207 (AAS), and 208;
aa) SEQ ID NOs: 210, 211, 212, 214, 215 (TAS), and 216;
bb) SEQ ID NOs: 218, 219, 220, 222, 223 (WAS), and 224;
cc) SEQ ID NOs: 226, 227, 228, 230, 231 (WAS), and 232;
dd) SEQ ID NOs: 234, 235, 236, 238, 239 (DAS), and 240;
ee) SEQ ID NOs: 242, 243, 244, 246, 247 (WAS), and 248;
ff) SEQ ID NOs: 250, 251, 252, 254, 25 (DAS)5, and 256;
gg) SEQ ID NOs: 258, 259, 260, 262, 263 (WAS), and 264;
hh) SEQ ID NOs: 266, 267, 268, 270, 271 (RDN), and 272;
ii) SEQ ID NOs: 274, 275, 276, 278, 279 (WAS), and 280;
jj) SEQ ID NOs: 282, 283, 284, 286, 287 (LGS), and 288;
kk) SEQ ID NOs: 290, 291, 292, 294, 295 (KDS), and 296;
ll) SEQ ID NOs: 298, 299, 300, 302, 303 (GAS), and 304;
mm) SEQ ID NOs: 306, 307, 308, 310, 311 (WAS), and 312;
nn) SEQ ID NOs: 314, 315, 316, 318, 319 (GAS), and 320;
oo) SEQ ID NOs: 322, 323, 324, 326, 327 (GAS), and 328;
pp) SEQ ID NOs: 330, 331, 332, 334, 335 (KDR), and 336;
qq) SEQ ID NOs: 338, 339, 340, 342, 343 (GAS), and 344;
rr) SEQ ID NOs: 346, 347, 348, 350, 351 (LGS), and 352;
ss) SEQ ID NOs: 354, 355, 356, 358, 359 (GAS), and 360;
tt) SEQ ID NOs: 362, 363, 364, 366, 367 (WAS), and 368;
uu) SEQ ID NOs: 370, 371, 372, 374, 375 (WAS), and 376;
vv) SEQ ID NOs: 378, 379, 380, 382, 383 (KAS), and 384;
ww) SEQ ID NOs: 386, 387, 388, 390, 391 (RAS), and 392;
xx) SEQ ID NOs: 394, 395, 396, 398, 399 (WAS) and 400;
yy) SEQ ID NOs: 402, 403, 404, 406, 407 (WAS), and 408;
zz) SEQ ID NOs: 410, 411, 412, 414, 415 (EVS), and 416;
aaa) SEQ ID NOs: 418, 419, 420, 422, 423 (KAS), and 424;
bbb) SEQ ID NOs: 426, 427, 428, 430, 431 (WAS), and 432;
ccc) SEQ ID NOs: 434, 435, 436, 438, 439 (EVT), and 440;
ddd) SEQ ID NOs: 442, 443, 444, 446, 447 (WAS), and 448;
eee) SEQ ID NOs: 450, 451, 452, 454, 455 (ATS), and 456;
fff) SEQ ID NOs: 458, 459, 460, 462, 463 (GAS), and 464;
ggg) SEQ ID NOs: 466, 467, 468, 470, 471 (WAS), and 472;
hhh) SEQ ID NOs: 474, 475, 476, 478, 479 (WAS), and 480; or
iii) SEQ ID NOs: 482, 483, 484, 486, 487 (LGS), and 488.

3. The antibody or antigen binding fragment of claim 1, wherein the V_{H} and the V_{L} respectively comprise amino acid sequences at least 90% identical to the amino acid sequences set forth as
a) SEQ ID NOs: 17 and 21;
b) SEQ ID NOs: 9 and 13;
c) SEQ ID NOs: 1 and 5;
d) SEQ ID NOs: 25 and 29;
e) SEQ ID NOs: 33 and 37;
f) SEQ ID NOs: 49 and 53;
g) SEQ ID NOs: 41 and 45;
h) SEQ ID NOs: 57 and 61;
i) SEQ ID NOs: 65 and 69;
j) SEQ ID NOs: 73 and 77;
k) SEQ ID NOs: 81 and 85;
l) SEQ ID NOs: 89 and 93;
m) SEQ ID NOs: 97 and 101;
n) SEQ ID NOs: 105 and 109;
o) SEQ ID NOs: 113 and 117;
p) SEQ ID NOs: 121 and 125;
q) SEQ ID NOs: 129 and 133;
r) SEQ ID NOs: 137 and 141;
s) SEQ ID NOs: 145 and 149;
t) SEQ ID NOs: 153 and 157;
u) SEQ ID NOs: 161 and 165;
v) SEQ ID NOs: 169 and 173;
w) SEQ ID NOs: 177 and 181;
x) SEQ ID NOs: 185 and 189;
y) SEQ ID NOs: 193 and 197;
z) SEQ ID NOs: 201 and 205;
aa) SEQ ID NOs: 209 and 213;
bb) SEQ ID NOs: 217 and 221;
cc) SEQ ID NOs: 225 and 229;
dd) SEQ ID NOs: 233 and 237;
ee) SEQ ID NOs: 241 and 245;
ff) SEQ ID NOs: 249 and 253;
gg) SEQ ID NOs: 257 and 261;
hh) SEQ ID NOs: 265 and 269;
ii) SEQ ID NOs: 273 and 277;
jj) SEQ ID NOs: 281 and 285;
kk) SEQ ID NOs: 289 and 293;
ll) SEQ ID NOs: 297 and 301;
mm) SEQ ID NOs: 305 and 309;
nn) SEQ ID NOs: 313 and 317;
oo) SEQ ID NOs: 321 and 325;
pp) SEQ ID NOs: 329 and 333;
qq) SEQ ID NOs: 337 and 341;
rr) SEQ ID NOs: 345 and 349;
ss) SEQ ID NOs: 353 and 357;
tt) SEQ ID NOs: 361 and 365;
uu) SEQ ID NOs: 369 and 373;
vv) SEQ ID NOs: 377 and 381;
ww) SEQ ID NOs: 385 and 389;
xx) SEQ ID NOs: 393 and 397;
yy) SEQ ID NOs: 401 and 405;
zz) SEQ ID NOs: 409 and 413;
aaa) SEQ ID NOs: 417 and 421;
bbb) SEQ ID NOs: 425 and 429;
ccc) SEQ ID NOs: 433 and 437;
ddd) SEQ ID NOs: 441 and 445;
eee) SEQ ID NOs: 449 and 453;
fff) SEQ ID NOs: 457 and 461;
ggg) SEQ ID NOs: 465 and 469;
hhh) SEQ ID NOs: 473 and 477; or
iii) SEQ ID NOs: 481 and 485.

4. The antibody or antigen binding fragment of claim 1, comprising a human framework region.

5. The antibody or antigen binding fragment of claim 1, wherein the V_{H} and the V_{L} respectively comprise the amino acid sequences set forth as:
a) SEQ ID NOs: 17 and 21;
b) SEQ ID NOs: 9 and 13;
c) SEQ ID NOs: 1 and 5;
d) SEQ ID NOs: 25 and 29;
e) SEQ ID NOs: 33 and 37;
f) SEQ ID NOs: 49 and 53;
g) SEQ ID NOs: 41 and 45;
h) SEQ ID NOs: 57 and 61;
i) SEQ ID NOs: 65 and 69;
j) SEQ ID NOs: 73 and 77;
k) SEQ ID NOs: 81 and 85;
l) SEQ ID NOs: 89 and 93;
m) SEQ ID NOs: 97 and 101;
n) SEQ ID NOs: 105 and 109;
o) SEQ ID NOs: 113 and 117;
p) SEQ ID NOs: 121 and 125;
q) SEQ ID NOs: 129 and 133;
r) SEQ ID NOs: 137 and 141;
s) SEQ ID NOs: 145 and 149;
t) SEQ ID NOs: 153 and 157;
u) SEQ ID NOs: 161 and 165;
v) SEQ ID NOs: 169 and 173;
w) SEQ ID NOs: 177 and 181;
x) SEQ ID NOs: 185 and 189;
y) SEQ ID NOs: 193 and 197;
z) SEQ ID NOs: 201 and 205;
aa) SEQ ID NOs: 209 and 213;
bb) SEQ ID NOs: 217 and 221;
cc) SEQ ID NOs: 225 and 229;
dd) SEQ ID NOs: 233 and 237;
ee) SEQ ID NOs: 241 and 245;
ff) SEQ ID NOs: 249 and 253;
gg) SEQ ID NOs: 257 and 261;
hh) SEQ ID NOs: 265 and 269;
ii) SEQ ID NOs: 273 and 277;
jj) SEQ ID NOs: 281 and 285;
kk) SEQ ID NOs: 289 and 293;
ll) SEQ ID NOs: 297 and 301;
mm) SEQ ID NOs: 305 and 309;
nn) SEQ ID NOs: 313 and 317;
oo) SEQ ID NOs: 321 and 325;
pp) SEQ ID NOs: 329 and 333;
qq) SEQ ID NOs: 337 and 341;
rr) SEQ ID NOs: 345 and 349;
ss) SEQ ID NOs: 353 and 357;
tt) SEQ ID NOs: 361 and 365;
uu) SEQ ID NOs: 369 and 373;
vv) SEQ ID NOs: 377 and 381;
ww) SEQ ID NOs: 385 and 389;
xx) SEQ ID NOs: 393 and 397;
yy) SEQ ID NOs: 401 and 405;
zz) SEQ ID NOs: 409 and 413;
aaa) SEQ ID NOs: 417 and 421;
bbb) SEQ ID NOs: 425 and 429;
ccc) SEQ ID NOs: 433 and 437;
ddd) SEQ ID NOs: 441 and 445;
eee) SEQ ID NOs: 449 and 453;
fff) SEQ ID NOs: 457 and 461;
ggg) SEQ ID NOs: 465 and 469;
hhh) SEQ ID NOs: 473 and 477; or
iii) SEQ ID NOs: 481 and 485.

6. The antibody of claim 1, wherein the antibody comprises a human constant domain.

7. The antibody of claim 1, wherein the antibody is a human antibody.

8. The antibody of claim 1, wherein the antibody is an IgG.

9. The antibody of claim 1, comprising a recombinant constant domain comprising a modification that increases the half-life of the antibody.

10. The antibody of claim 9, wherein the modification increases binding to the neonatal Fc receptor.

11. The antibody or antigen binding fragment of claim 1, wherein the antibody specifically binds an N-terminal domain of the coronavirus spike.

12. The antibody or antigen binding fragment of claim 1, wherein the antibody specifically binds a S domain of the coronavirus spike protein.

13. The antibody or antigen binding fragment of claim 1, wherein the antibody neutralizes SARS-CoV-2.

14. The antigen binding fragment of claim 1.

15. The antigen binding fragment of claim 14, wherein the antigen binding fragment is a Fv, Fab, F(ab′)₂, scFV or a scFV₂ fragment.

16. The antibody or antigen binding of claim 1, wherein the antibody or antigen binding fragment specifically binds a spike protein from at least three betacoronaviruses selected from the group consisting of SARS-CoV-2, SARS-CoV, MERS-CoV, HKU1, and OC43.

17. (canceled)

18. The antibody or antigen binding of claim 1, wherein the antibody or antigen binding fragment specifically binds a spike protein from SARS-CoV-2, SARS-CoV, MERS-CoV, HKU1, OC43, NL63 and 229E.

19. The antibody or antigen binding of claim 1, wherein the antibody or antigen binding fragment specifically binds a stem-helix in the S2 domain of the spike protein.

20. The antibody or antigen binding fragment of claim 1, conjugated to a detectable marker.

21. A bispecific antibody comprising the antibody or antigen binding fragment of claim 1.

22. An isolated nucleic acid molecule encoding the antibody or antigen binding fragment of claim 1, or a V_{H} or V_{L} of the antibody.

23. The nucleic acid molecule of claim 22, wherein the nucleic acid molecule is a cDNA sequence encoding the V_{H} or V_{L}.

24. The nucleic acid molecule of claim 22, operably linked to a promoter.

25. A vector comprising the nucleic acid molecule of claim 22.

26. A host cell comprising the nucleic acid molecule of claim 22.

27. A pharmaceutical composition for use in inhibiting a coronavirus infection, comprising an effective amount of the antibody or antigen binding fragment of claim 1, a bispecific antibody comprising the antibody or antigen binding fragment, a nucleic acid molecule encoding the antibody or antigen binding fragment, or a vector comprising the nucleic acid molecule; and
a pharmaceutically acceptable carrier.

28. A method of producing an antibody or antigen binding fragment that specifically binds to a coronavirus spike protein, comprising:
expressing one or more nucleic acid molecules encoding the antibody, antigen binding fragment of claim 1 in a host cell; and
purifying the antibody or antigen binding fragment.

29. A method of detecting the presence of a coronavirus in a biological sample from a subject, comprising: contacting the biological sample with an effective amount of the antibody or antigen binding fragment of claim 1 under conditions sufficient to form an immune complex; and
detecting the presence of the immune complex in the biological sample, wherein the presence of the immune complex in the biological sample indicates the presence of the coronavirus in the sample.

30. The method of claim 29, wherein detecting the presence of the immune complex in the biological sample indicates that the subject has a SARS-CoV-2, SARS-CoV, MERS-CoV, OC43, NL63, 229E, or HKU1 infection.

31. The method of claim 29, wherein detecting the presence of the immune complex in the biological sample indicates that the subject has a SARS-CoV-2 infection.

32. A method of inhibiting a coronavirus infection in a subject, comprising administering an effective amount of the antibody, antigen binding fragment, nucleic acid molecule, vector, or pharmaceutical composition of claim 1 to the subject, wherein the subject has or is at risk of a coronavirus infection.

33. The method of claim 32, wherein the coronavirus is SARS-CoV-2, SARS-CoV, MERS-CoV, OC43, NL63, 229E, or HKU1.

34. The method of claim 32, wherein the coronavirus is SARS-CoV-2.

35-37. (canceled)


SEQUENCE LISTING (532 sequences)

SEQ ID NO: 1 - protein, 118 aa, Homo sapiens
QEQLVQSGAEVKKPGASVKVSCKSSGFTFSYFYLHWVRQAPGQGLEWMGIINPRGDGTRYAQKFQGRVTMTRDASTGTLYMELRSLRSEDTAVYYCARGADHGAFDIWGQGTMVTVSS

SEQ ID NO: 2 - protein, 8 aa, Homo sapiens
GFTFSYFY

SEQ ID NO: 3 - protein, 8 aa, Homo sapiens
INPRGDGT

SEQ ID NO: 4 - protein, 11 aa, Homo sapiens
ARGADHGAFDI

SEQ ID NO: 5 - protein, 110 aa, Homo sapiens
EIVLTQSPGTLSLSPGERATLSCRASQSVRRNYFAWYQQKRGQAPRLLIYDASTRATGIPDRFSGSGSGTDFTLTISRLEPEDFAVYYCQQYDSSPPMYIFGQGTKLEIK

SEQ ID NO: 6 - protein, 7 aa, Homo sapiens
QSVRRNY

SEQ ID NO: 7 - skipped

SEQ ID NO: 8 - protein, 11 aa, Homo sapiens
QQYDSSPPMYI

SEQ ID NO: 9 - protein, 124 aa, Homo sapiens
QMQLMQSGAEVKKPGASVTVSCKASGDTFSDYRIHWVRQAPGQGLEWMGRMNPKSGDTNFAQKFQGRVTMTRDMSINTAYMTLSGLTFDDTALYYCASLLIVGGFDPLDDFEVWGQGTMVTISS

SEQ ID NO: 10 - protein, 8 aa, Homo sapiens
GDTFSDYR

SEQ ID NO: 11 - protein, 8 aa, Homo sapiens
MNPKSGDT

SEQ ID NO: 12 - protein, 17 aa, Homo sapiens
ASLLIVGGFDPLDDFEV

SEQ ID NO: 13 - protein, 110 aa, Homo sapiens
QSALTQPPSASGSPGQSVTISCSGTSSDVGGYNFVSWYQHHPGKAPKILIYEVTKRPSGVPDRFSGSKSGNTASLTVSGLQAEDEADYYCSSYGGTNNLLFGGGTKLTVL

SEQ ID NO: 14 - protein, 9 aa, Homo sapiens
SSDVGGYNF

SEQ ID NO: 15 - skipped

SEQ ID NO: 16 - protein, 10 aa, Homo sapiens
SSYGGTNNLL

SEQ ID NO: 17 - protein, 126 aa, Homo sapiens [sequence omitted]

SEQ ID NO: 18 - protein, 8 aa, Homo sapiens
GLTFSGYA

SEQ ID NO: 19 - protein, 8 aa, Homo sapiens
ISRDARNK

SEQ ID NO: 20 - protein, 19 aa, Homo sapiens
AILIIPGITEPGSPDALDI

SEQ ID NO: 21 - protein, 109 aa, Homo sapiens [sequence omitted]

SEQ ID NO: 22 - protein, 6 aa, Homo sapiens
QDISKW

SEQ ID NO: 23 - skipped

SEQ ID NO: 24 - protein, 11 aa, Homo sapiens
QQASSFPWSIT

SEQ ID NO: 25 - protein, 118 aa, Homo sapiens
QVQLVQSGAEVKKPGASVKVSCQTSGYTFTSYYMHWVRQAPGQGLEWMGLITPSGDDTYYAQRFQGRVTMTRDTSTSPTYMELSSLTSEDTAVYYCAKMSRAGGFDVWGQGTLVTVSS

SEQ ID NO: 26 - protein, 8 aa, Homo sapiens
GYTFTSYY

SEQ ID NO: 27 - protein, 8 aa, Homo sapiens
ITPSGDDT

SEQ ID NO: 28 - protein, 11 aa, Homo sapiens
AKMSRAGGFDV

SEQ ID NO: 29 - protein, 110 aa, Homo sapiens
EVVLTQSPGTLSLSPGERATLSCRASQSITGRYLAWYQQKPGQAPRLLMYGESSRVTGIPDRFSGGGSGTDFTLTISRLEPEDFAVYYCQHFASSPPTYTFGQGTKLEIR

SEQ ID NO: 30 - protein, 7 aa, Homo sapiens
QSITGRY

SEQ ID NO: 31 - skipped

SEQ ID NO: 32 - protein, 11 aa, Homo sapiens
QHFASSPPTYT

SEQ ID NO: 33 - protein, 117 aa, Homo sapiens
QVQLVQSGAEVKKPAASVKVSCKASGDTFSSHYMHWVRQAPGQGPEWMGIINPSGSGTAYGQKFQGRLTMTRDTSTSTVYMELSSLTSDDTAVYYCGGGSGGLFAYWGQGTLVTVSS

SEQ ID NO: 34 - protein, 8 aa, Homo sapiens
GDTFSSHY

SEQ ID NO: 35 - protein, 8 aa, Homo sapiens
INPSGSGT

SEQ ID NO: 36 - protein, 10 aa, Homo sapiens
GGGSGGLFAY

SEQ ID NO: 37 - protein, 110 aa, Homo sapiens
EIVLTQSPGTLSLSPGERATLSCRASQIVRSNYLAWYQQKPGQAPRLLIYGASSRATGTPDRFSGGGSGTDFTLTINRLEPEDFAVYYCLQYDSSPPTYIFGQGTKLEIK

SEQ ID NO: 38 - protein, 7 aa, Homo sapiens
QIVRSNY

SEQ ID NO: 39 - skipped

SEQ ID NO: 40 - protein, 11 aa, Homo sapiens
LQYDSSPPTYI

SEQ ID NO: 41 - protein, 115 aa, Homo sapiens
QLQLVQSGAEVKKPGASVKLSCKASGNTFTSHYIHWVRQAPGQGPEWMGIINPSGSGTRYAQKFQGRVTMTRDTSTSTVYMELVSLRFEDTAVYYCAGDYYLDYWGQGTLVTVSS

SEQ ID NO: 42 - protein, 8 aa, Homo sapiens
GNTFTSHY

SEQ ID NO: 43 - protein, 8 aa, Homo sapiens
INPSGSGT

SEQ ID NO: 44 - protein, 8 aa, Homo sapiens
AGDYYLDY

SEQ ID NO: 45 - protein, 110 aa, Homo sapiens
EFVLTQSPGTLSLSPGERATLSCRASQSVTSNYLAWYQQKPGQAPRLLIFGASSRATGIPDRFSGSGSGTDFTLTISRLEPEDFAVYYCQQYGSSPPMYTFGPGTKVEIK

SEQ ID NO: 46 - protein, 7 aa, Homo sapiens
QSVTSNY

SEQ ID NO: 47 - skipped

SEQ ID NO: 48 - protein, 11 aa, Homo sapiens
QQYGSSPPMYT

SEQ ID NO: 49 - protein, 121 aa, Homo sapiens
QVQLVESGGGVVHPGGSLRLSCAASGFTFSDFAMNWVRQAPGEGLQWVAIISYDGRNKHFAASVRGRFTISRDNSKNTMFLQMSSLRAEDTAIYYCATLGGWFEEASPTYWGRGTLVTVSS

SEQ ID NO: 50 - protein, 8 aa, Homo sapiens
GFTFSDFA

SEQ ID NO: 51 - protein, 8 aa, Homo sapiens
ISYDGRNK

SEQ ID NO: 52 - protein, 14 aa, Homo sapiens
ATLGGWFEEASPTY

SEQ ID NO: 53 - protein, 108 aa, Homo sapiens
EVVLTQSPAIVSLSPGERATLSCRASQSVGSRLAWHQQKPGLAPRVLIYDASNRATGIPARFSGSGSGTDFTLTISSIEPEDSAVYYCQQRGDWPLITFGQGTRLEIK

SEQ ID NO: 54 - protein, 6 aa, Homo sapiens
QSVGSR

SEQ ID NO: 55 - skipped

SEQ ID NO: 56 - protein, 10 aa, Homo sapiens
QQRGDWPLIT

SEQ ID NO: 57 - protein, 114 aa, Homo sapiens
QVQLVQSGAEAKKPGASVKVSCKASGYSFTSYHMHWVRQAPGQGLEWMGIINPNGGTTTYAPKFQGRVTMTSDTSTSTVYMELTSLRSEDTAVYYCSTVVGALWGQGTLVIVSS

SEQ ID NO: 58 - protein, 8 aa, Homo sapiens
GYSFTSYH

SEQ ID NO: 59 - protein, 8 aa, Homo sapiens
INPNGGTT

SEQ ID NO: 60 - protein, 7 aa, Homo sapiens
STVVGAL

SEQ ID NO: 61 - protein, 108 aa, Homo sapiens
IVLTQSPGTLSLSPGERATLSCRASQSVRSNNYLAWYQQKPGQAPRLLIYGASTRATGIPDRFSGSGSGTDFTLTISRLEPEDFAVYYCQQYGSSPLTFGGGTRVEIK

SEQ ID NO: 62 - protein, 8 aa, Homo sapiens
QSVRSNNY

SEQ ID NO: 63 - skipped

SEQ ID NO: 64 - protein, 9 aa, Homo sapiens
QQYGSSPLT

SEQ ID NO: 65 - protein, 118 aa, Homo sapiens
QVQLVQSGAEVKKPGASVKVSCKASGYTFSSYYMNWVRQAPGQGLEWVGLISPSAGYTAYAQMFQGRVTMTRDTSTSTVYMELSSLRSEDTAVYYCARVAVAGPNDYWGQGTLVTVSS

SEQ ID NO: 66 - protein, 8 aa, Homo sapiens
GYTFSSYY

SEQ ID NO: 67 - protein, 8 aa, Homo sapiens
ISPSAGYT

SEQ ID NO: 68 - protein, 11 aa, Homo sapiens
ARVAVAGPNDY

SEQ ID NO: 69 - protein, 110 aa, Homo sapiens
EIVLTQSPGTLSLSPGERATLSCRASQSISSSYLAWYQQKPGQAPRLVILAASTRATGIPDRFSGSGSGTDFTLTISRLEPEDFALYYCQHYRSSPPLWTFGQGTKVEIR

SEQ ID NO: 70 - protein, 7 aa, Homo sapiens
QSISSSY

SEQ ID NO: 71 - skipped

SEQ ID NO: 72 - protein, 11 aa, Homo sapiens
QHYRSSPPLWT

SEQ ID NO: 73 - protein, 118 aa, Homo sapiens
QVLLVQSGAEVKKPGASVMVSCKASGYILNNYYMHWVRQAPGHGLEWMGIINPSGDGTRYAQKFQGRVTMTRDSSTTSIHMELRRVTSEDTAVYYCARGGSQSAFDMWGQGTVVLVSS

SEQ ID NO: 74 - protein, 8 aa, Homo sapiens
GYILNNYY

SEQ ID NO: 75 - protein, 8 aa, Homo sapiens
INPSGDGT

SEQ ID NO: 76 - protein, 11 aa, Homo sapiens
ARGGSQSAFDM

SEQ ID NO: 77 - protein, 114 aa, Homo sapiens
DIVMTQSPRSLPVTPGESASISCRSSQSLLQKNGYNYLDWYVQKPGQSPQLLIYLGSYRASGVPDRISGSGSGTDFTLKINRVEAEDVGVYYCLQAQQAPPLYTFGQGTKLQIE

SEQ ID NO: 78 - protein, 11 aa, Homo sapiens
QSLLQKNGYNY

SEQ ID NO: 79 - skipped

SEQ ID NO: 80 - protein, 11 aa, Homo sapiens
LQAQQAPPLYT

SEQ ID NO: 81 - protein, 118 aa, Homo sapiens
QVQLVQSGAEVKKPGASVKVSCKASGYTFSNFYMNWVRQAPGQGLEWVGLISPSAGYTAYAQMFQGRVTMTRDTSTSTVYMELSSLRSEDTAVYYCARVAVAGPNDYWGQGTLVTVSS

SEQ ID NO: 82 - protein, 8 aa, Homo sapiens
GYTFSNFY

SEQ ID NO: 83 - protein, 8 aa, Homo sapiens
ISPSAGYT

SEQ ID NO: 84 - protein, 11 aa, Homo sapiens
ARVAVAGPNDY

SEQ ID NO: 85 - protein, 110 aa, Homo sapiens
EIVLTQSPGTLSLSPGERATLSCRASQSISSSYLAWYQQKPGQAPRLVIFAASTRATGIPDRFSGSGSGTDFTLTITRLEPEDFALYYCQHYRSSPPLWTFGQGTKVEIR

SEQ ID NO: 86 - protein, 7 aa, Homo sapiens
QSISSSY

SEQ ID NO: 87 - skipped

SEQ ID NO: 88 - protein, 11 aa, Homo sapiens
QHYRSSPPLWT

SEQ ID NO: 89 - protein, 118 aa, Homo sapiens
QVQLVQSGAEVKKPGASVRVSCKTSGYTFTNYYMTWVRQAPGQGLQWVGLISPSAGYTAYAQMFQGRVTMTSDTSTSTVYMQLSSLRSEDTAIYYCTRVAVAGPNDYWGQGTLVTVSS

SEQ ID NO: 90 - protein, 8 aa, Homo sapiens
GYTFTNYY

SEQ ID NO: 91 - protein, 8 aa, Homo sapiens
ISPSAGYT

SEQ ID NO: 92 - protein, 11 aa, Homo sapiens
TRVAVAGPNDY

SEQ ID NO: 93 - protein, 110 aa, Homo sapiens
EIVLTQSPGTLSLSPGERATLSCRASQSISGSYLAWYQQKPGQAPRLVIYAASSRATGIPDRFSGSGSGTDFILTISRLEAEDFAVYFCQHYRSSPPLWTFGQGTKVEIR

SEQ ID NO: 94 - protein, 7 aa, Homo sapiens
QSISGSY

SEQ ID NO: 95 - skipped

SEQ ID NO: 96 - protein, 11 aa, Homo sapiens
QHYRSSPPLWT

SEQ ID NO: 97 - protein, 125 aa, Homo sapiens
QVTLKESGPVLVKPTETLTLTCTVSGFSLSNTKMGVSWIRQPPGKALEWLAHIFSNDRKSYTTSLKSRLTISEDTSKSQIVLTMTNMDPVDTATYYCARIGAGGTYNFYYYMDVWGKGTTVTVSS

SEQ ID NO: 98 - protein, 10 aa, Homo sapiens
GFSLSNTKMG

SEQ ID NO: 99 - protein, 7 aa, Homo sapiens
IFSNDRK

SEQ ID NO: 100 - protein, 17 aa, Homo sapiens
ARIGAGGTYNFYYYMDV

SEQ ID NO: 101 - protein, 106 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSLYYTFGQGTKLEIK

SEQ ID NO: 102 - protein, 6 aa, Homo sapiens
QSVSSY

SEQ ID NO: 103 - skipped

SEQ ID NO: 104 - protein, 8 aa, Homo sapiens
QQRSLYYT

SEQ ID NO: 105 - protein, 125 aa, Homo sapiens
DVQLLESGGGLVQPGGSRRLSCAASGFTFSNYAMSWVRQAPGKGLEWVSAISGGGATTYYADSVQGRFTMSRDNSNNTLYLQMSSLRAEDTAVYYCTKGAFYDILSSIIVEGDYWGQGTLVTVSS

SEQ ID NO: 106 - protein, 7 aa, Homo sapiens
FTFSNYA

SEQ ID NO: 107 - protein, 8 aa, Homo sapiens
ISGGGATT

SEQ ID NO: 108 - protein, 18 aa, Homo sapiens
TKGAFYDILSSIIVEGDY

SEQ ID NO: 109 - protein, 113 aa, Homo sapiens
DVVLTQSPLSLPITLGQPASISCRSSQSLVFSDGNTYLNWFQQRPGQSPRRLIYKVSNRDSGVPDRFSGSGSGTDFTLKISRVEAEDVGVYYCMQGTHWLSITFGQGTRLEIK

SEQ ID NO: 110 - protein, 11 aa, Homo sapiens
QSLVFSDGNTY

SEQ ID NO: 111 - skipped

SEQ ID NO: 112 - protein, 10 aa, Homo sapiens
MQGTHWLSIT

SEQ ID NO: 113 - protein, 125 aa, Homo sapiens
EVQLLESGGGLVQPGGSLRLSCATSGFTFSSYAMTWVRQAPGKGLEWVSAISGGGATTYSTDSVKGRFTTSRDNSKNTLYLQMNNLRAEDTALYYCAKGAFYNILSSIIVEGDYWGQGTLVTVSS

SEQ ID NO: 114 - protein, 8 aa, Homo sapiens
GFTFSSYA

SEQ ID NO: 115 - protein, 8 aa, Homo sapiens
ISGGGATT

SEQ ID NO: 116 - protein, 18 aa, Homo sapiens
AKGAFYNILSSIIVEGDY

SEQ ID NO: 117 - protein, 113 aa, Homo sapiens
DVVMTQSPLSLSVTLGQPASISCRSSQSLVFSDGDTYLNWFQQRPGQSPRRLIYRVSNRDSGVPDRFSGSGSGTDFTLKISRVEAEDVGVYYCMQGTHWLSITFGQGTRLDIK

SEQ ID NO: 118 - protein, 11 aa, Homo sapiens
QSLVFSDGDTY

SEQ ID NO: 119 - skipped

SEQ ID NO: 120 - protein, 10 aa, Homo sapiens
MQGTHWLSIT

SEQ ID NO: 121 - protein, 125 aa, Homo sapiens
EVQLLESGGGLAQPGGSLRLSCVASGFSFNDYAMTWVRQAPGKGLEWVSAVSSGGQTTYSTDSVKGRFTISRDNSKNMLYLQMNSLRAEDTAVYYCVKGAFYNILSSIIVEGDYWGQGTLVTVSS

SEQ ID NO: 122 - protein, 8 aa, Homo sapiens
GFSFNDYA

SEQ ID NO: 123 - protein, 8 aa, Homo sapiens
VSSGGQTT

SEQ ID NO: 124 - protein, 18 aa, Homo sapiens
VKGAFYNILSSIIVEGDY

SEQ ID NO: 125 - protein, 113 aa, Homo sapiens
GVVLTQSPLSLPVTLGQPASISCRSSQSLVFSDGNTYLNWFQQRPGQSPRRLIYQVSNRDPGVPDRFSGSGSGTDFTLKISRVEAEDVGVYYCMQGTHWLSITFGQGTRLEIK

SEQ ID NO: 126 - protein, 11 aa, Homo sapiens
QSLVFSDGNTY

SEQ ID NO: 127 - skipped

SEQ ID NO: 128 - protein, 10 aa, Homo sapiens
MQGTHWLSIT

SEQ ID NO: 129 - protein, 126 aa, Homo sapiens
EVQLLESGGGLVRPGGSLRLSCAASGFTFTNYAMSWVRQAPGKGLEWVSAISGGDGRTSYYADSVKGRFTSSRDNSKNTLYLQMNSLRAEDTAVYYCAKGAFYNILSSIIVEGDYWGQGTLVTVSS

SEQ ID NO: 130 - protein, 8 aa, Homo sapiens
GFTFTNYA

SEQ ID NO: 131 - protein, 9 aa, Homo sapiens
ISGGDGRTS

SEQ ID NO: 132 - protein, 18 aa, Homo sapiens
AKGAFYNILSSIIVEGDY

SEQ ID NO: 133 - protein, 113 aa, Homo sapiens
DVVMTQSPLSLPVTLGQPASISCRSSQSLVFSDGNTYLNWFQQRPGQSPRRLIYKVSNRDSGVPDRFSGSGSGTDFTLKISRVEAEDVGVYYCMQGTHWLSITFGQGTRLEIR

SEQ ID NO: 134 - protein, 11 aa, Homo sapiens
QSLVFSDGNTY

SEQ ID NO: 135 - skipped

SEQ ID NO: 136 - protein, 10 aa, Homo sapiens
MQGTHWLSIT

SEQ ID NO: 137 - protein, 125 aa, Homo sapiens
EVQLLESGGGLVQPGGSLRVSCTVSGFTFSDYSMSWVRQAPGKGLEWVSAISGTGETAYYADFVKGRFTSSRDNSKNTLYLQMNTLRGEDTAVYYCVKGAFYNILSSIIVEGDYWGQGTLVTVSS

SEQ ID NO: 138 - protein, 8 aa, Homo sapiens
GFTFSDYS

SEQ ID NO: 139 - protein, 8 aa, Homo sapiens
ISGTGETA

SEQ ID NO: 140 - protein, 18 aa, Homo sapiens
VKGAFYNILSSIIVEGDY

SEQ ID NO: 141 - protein, 113 aa, Homo sapiens
GVVMTQSPLSLPVTLGQPASISCRSSQSLVFSDGNTYLNWFQQRPGQSPRRLIYKVSERDSGVPDRFSGSGSGTDFTLTISRVEAEDVGVYYCMQGTHWLSITFGQGTRLEIK

SEQ ID NO: 142 - protein, 11 aa, Homo sapiens
QSLVFSDGNTY

SEQ ID NO: 143 - skipped

SEQ ID NO: 144 - protein, 10 aa, Homo sapiens
MQGTHWLSIT

SEQ ID NO: 145 - protein, 125 aa, Homo sapiens
EVQLLESGGGLVQPGGSLRVSCAVSGFTFSNYAMSWVRQAPGKGLDWVSAISGSGETRYYADFVKGRFTISRDNSKNTLYLQMNSLRGEDTAVYYCVKGAFYNILSSIIVEGDYWGQGTLVTVSS

SEQ ID NO: 146 - protein, 8 aa, Homo sapiens
GFTFSNYA

SEQ ID NO: 147 - protein, 8 aa, Homo sapiens
ISGSGETR

SEQ ID NO: 148 - protein, 18 aa, Homo sapiens
VKGAFYNILSSIIVEGDY

SEQ ID NO: 149 - protein, 113 aa, Homo sapiens
DVVMTQSPLSLPVTLGQPASISCRSSQSLVFSDGNTYLNWFQQRPGQSPRRLIYKVSKRDSGVPDRFSGSGSGTDFTLKISRVEAEDVGVYYCMQGTHWLSITFGQGTRLEIK

SEQ ID NO: 150 - protein, 11 aa, Homo sapiens
QSLVFSDGNTY

SEQ ID NO: 151 - skipped

SEQ ID NO: 152 - protein, 10 aa, Homo sapiens
MQGTHWLSIT

SEQ ID NO: 153 - protein, 125 aa, Homo sapiens
EVQLLESGGGLAQPGGSLRISCVASGFSFNDYAMAWVRRAPGKGLEWVSAVSGGGQTTYSTDSVKGRFTTSRDNSKNTLYLQMNGLRAEDTAVYYCVKGAFYNILSSIIVEGDYWGQGALVTVSS

SEQ ID NO: 154 - protein, 8 aa, Homo sapiens
GFSFNDYA

SEQ ID NO: 155 - protein, 8 aa, Homo sapiens
VSGGGQTT

SEQ ID NO: 156 - protein, 18 aa, Homo sapiens
VKGAFYNILSSIIVEGDY

SEQ ID NO: 157 - protein, 113 aa, Homo sapiens; identical to SEQ ID NO: 125

SEQ ID NO: 158 - protein, 11 aa, Homo sapiens
QSLVFSDGNTY

SEQ ID NO: 159 - skipped

SEQ ID NO: 160 - protein, 10 aa, Homo sapiens
MQGTHWLSIT

SEQ ID NO: 161 - protein, 125 aa, Homo sapiens
GVQLLESGGGLAQPGGSLKLSCVASGFSFNDYAMTWVRRPPGKGLEWVSAVSSGGQTSYSTDSVKGRFTISRDNSKNTLYLQMSSLRAEDTAIYYCVKGAFYNILSSIIVEGDYWGQGTLVTVSS

SEQ ID NO: 162 - protein, 8 aa, Homo sapiens
GFSFNDYA

SEQ ID NO: 163 - protein, 8 aa, Homo sapiens
VSSGGQTS

SEQ ID NO: 164 - protein, 18 aa, Homo sapiens
VKGAFYNILSSIIVEGDY

SEQ ID NO: 165 - protein, 113 aa, Homo sapiens
GVVLTQSPLSLPVTLGQPASISCRSSQSLVFSDGKTYLNWFQQRPGQSPRRLIYQVSNRDPGVPDRFSGSGSGTDFTLKISRVEAEDVGVYYCMQGTHWLSITFGQGTRLEIK

SEQ ID NO: 166 - protein, 11 aa, Homo sapiens
QSLVFSDGKTY

SEQ ID NO: 167 - skipped

SEQ ID NO: 168 - protein, 10 aa, Homo sapiens
MQGTHWLSIT

SEQ ID NO: 169 - protein, 125 aa, Homo sapiens
EVQLLESGGGLVQPGGSLRVSCTVSGFTFSDYSMSWVRQAPGKGLDWVSAISGTGETAYYADFVKGRFTSSRDNSKNTVYLQMNSLRGEDTAVYYCVKGVFYNILSSIIVEGDYWGQGTLVTVSS

SEQ ID NO: 170 - protein, 8 aa, Homo sapiens
GFTFSDYS

SEQ ID NO: 171 - protein, 8 aa, Homo sapiens
ISGTGETA

SEQ ID NO: 172 - protein, 18 aa, Homo sapiens
VKGVFYNILSSIIVEGDY

SEQ ID NO: 173 - protein, 113 aa, Homo sapiens
GVVMTQSPLSLPVTLGQPASISCRSSQSLVFSDGNTYLNWFQQRPGQSPRRLIYKVSKRDSGVPDRFSGSGSGTDFTLTISRVEAEDVGVYYCMQGTHWLSITFGQGTRLEIK

SEQ ID NO: 174 - protein, 11 aa, Homo sapiens
QSLVFSDGNTY

SEQ ID NO: 175 - skipped

SEQ ID NO: 176 - protein, 10 aa, Homo sapiens
MQGTHWLSIT

SEQ ID NO: 177 - protein, 125 aa, Homo sapiens
EVQLLESGGGLAQPGGSLRLSCVASGFSFDDYAMTWVRQPPGKGLEWVSAVSGGGQTTYSTDSVKGRFTTSRDNSKNTLYLQMNSLRGEDTAVYYCVKGAFYNILSSIIVEGDYWGQGTLVTVSS

SEQ ID NO: 178 - protein, 8 aa, Homo sapiens
GFSFDDYA

SEQ ID NO: 179 - protein, 8 aa, Homo sapiens
VSGGGQTT

SEQ ID NO: 180 - protein, 18 aa, Homo sapiens
VKGAFYNILSSIIVEGDY

SEQ ID NO: 181 - protein, 113 aa, Homo sapiens
VVVLTQSPLSLPVTLGQPASVSCRSSQSLVFSDGNTYLNWFQQRPGQSPRRLIYQVSNRDPGVPDRFSGSGSGTDFTLKISRVEAEDVGVYYCMQGTHWLSITFGQGTRLEIK

SEQ ID NO: 182 - protein, 11 aa, Homo sapiens
QSLVFSDGNTY

SEQ ID NO: 183 - skipped

SEQ ID NO: 184 - protein, 10 aa, Homo sapiens
MQGTHWLSIT

SEQ ID NO: 185 - protein, 123 aa, Homo sapiens
QVHLVQSGAEVKNPGVSVKVSCKASGYTFTTSGITWVRQAPGQGLEWMGWISTYNGNTETAQTFQGRVTMTTDTSTSTAYMELRRLRSDDTAVYYCARVQSVATSSNYFFDYWGQGTRVTVSS

SEQ ID NO: 186 - protein, 8 aa, Homo sapiens
GYTFTTSG

SEQ ID NO: 187 - protein, 8 aa, Homo sapiens
ISTYNGNT

SEQ ID NO: 188 - protein, 16 aa, Homo sapiens
ARVQSVATSSNYFFDY

SEQ ID NO: 189 - protein, 109 aa, Homo sapiens
DIQMTQSPSSLSASVGDRVTITCRASQTIDRFLNWYQQKPGKAPKLLIYAASRLQSGVPSRFSGSGSGTTYTLTISSLQPEDFATYFCQQSYSDPRALTFGGGTKVEIK

SEQ ID NO: 190 - protein, 6 aa, Homo sapiens
QTIDRF

SEQ ID NO: 191 - skipped

SEQ ID NO: 192 - protein, 11 aa, Homo sapiens
QQSYSDPRALT

SEQ ID NO: 193 - protein, 125 aa, Homo sapiens
EVQLLESGGGLAQPGGSLRLSCVASGFSFNDYAMAWVRQAPGKGLEWVSAVSGGGQTTYSTDSVKGRFTTSRDNSKNTLYLQMNGLRAEDAAVYYCVKGAFYNILSSIIVEGDNWGQGTLVTVSS

SEQ ID NO: 194 - protein, 8 aa, Homo sapiens
GFSFNDYA

SEQ ID NO: 195 - protein, 8 aa, Homo sapiens
VSGGGQTT

SEQ ID NO: 196 - protein, 18 aa, Homo sapiens
VKGAFYNILSSIIVEGDN

SEQ ID NO: 197 - protein, 113 aa, Homo sapiens; identical to SEQ ID NO: 125

SEQ ID NO: 198 - protein, 11 aa, Homo sapiens
QSLVFSDGNTY

SEQ ID NO: 199 - skipped

SEQ ID NO: 200 - protein, 10 aa, Homo sapiens
MQGTHWLSIT

SEQ ID NO: 201 - protein, 113 aa, Homo sapiens
EVQLVESGGGLVQPGGSLRLSCAASGFIVSSNYMSWVRQAPGKGLEWVSMIYSGGSTVYADSVKGRFTISRDISKNTLYLQMNSLRAEDTAVYYCALETFDYWGQGTLVTVSS

SEQ ID NO: 202 - protein, 8 aa, Homo sapiens
GFIVSSNY

SEQ ID NO: 203 - protein, 7 aa, Homo sapiens
IYSGGST

SEQ ID NO: 204 - protein, 7 aa, Homo sapiens
ALETFDY

SEQ ID NO: 205 - protein, 107 aa, Homo sapiens
DIQMTQSPSSLSASVGDRVTITCRASQGITNHLAWYQQKPGKVPKLLIYAASTLQSGVPSRFSGSGYGTDFTLTISSLQPEDVASYYCQKYDSAPRTFGQGTKVEIK

SEQ ID NO: 206 - protein, 6 aa, Homo sapiens
QGITNH

SEQ ID NO: 207 - skipped

SEQ ID NO: 208 - protein, 9 aa, Homo sapiens
QKYDSAPRT

SEQ ID NO: 209 - protein, 126 aa, Homo sapiens
QVQLVESGGGVVQPGRSLRLSCTVSGFTFNNYGMHWVRQAPGKGLEWLAVISYEGSKKFYADSLEGRFSISRDSSKNTLYLQMNSLTIEDTAVYFCAKRNSIFWFGEGRKDAFDVWGQGTRVIVSS

SEQ ID NO: 210 - protein, 8 aa, Homo sapiens
GFTFNNYG

SEQ ID NO: 211 - protein, 8 aa, Homo sapiens
ISYEGSKK

SEQ ID NO: 212 - protein, 19 aa, Homo sapiens
AKRNSIFWFGEGRKDAFDV

SEQ ID NO: 213 - protein, 108 aa, Homo sapiens
DIQLTQSPSSLSASVGDRVTITCRASQTVDNYLNWYQQLPGKAPKLLIHTASTLQSGVPSRFSATSSGTDFTLTISGLQPDDSATYFCQQSYSVPLTFGGGTKVEIKV

SEQ ID NO: 214 - protein, 6 aa, Homo sapiens
QTVDNY

SEQ ID NO: 215 - skipped

SEQ ID NO: 216 - protein, 9 aa, Homo sapiens
QQSYSVPLT

SEQ ID NO: 217 - protein, 124 aa, Homo sapiens
QVHLVESGGGVVQPGRSLRLSCAASGFTFSSFGMHWVRQAPGKGLEWVAVISDDGNNKFYADSVKGRFSISRDNSKNTLYLQMNSLRAEDTAVYYCVRAFRNSNWATGEYFDYWGQGTLVTVSS

SEQ ID NO: 218 - protein, 8 aa, Homo sapiens
GFTFSSFG

SEQ ID NO: 219 - protein, 8 aa, Homo sapiens
ISDDGNNK

SEQ ID NO: 220 - protein, 17 aa, Homo sapiens
VRAFRNSNWATGEYFDY

SEQ ID NO: 221 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFHRIPPTFGQGTKVEIK

SEQ ID NO: 222 - protein, 12 aa, Homo sapiens
QSVLYSSNNKNY

SEQ ID NO: 223 - skipped

SEQ ID NO: 224 - protein, 9 aa, Homo sapiens
QQFHRIPPT

SEQ ID NO: 225 - protein, 124 aa, Homo sapiens
QVQLVESGGGVVQPGRSLRLSCAASGFTFSNFGMHWVRQAPGKGLEWVAVISDDGNNEFYAGSVKGRFSISRDNSKNTLYLQMNSLRPEDTAVYYCVRAFRNSNWATGEYFDYWGQGTLVTVSS

SEQ ID NO: 226 - protein, 8 aa, Homo sapiens
GFTFSNFG

SEQ ID NO: 227 - protein, 8 aa, Homo sapiens
ISDDGNNE

SEQ ID NO: 228 - protein, 14 aa, Homo sapiens
VRAFRNSNWATGEY

SEQ ID NO: 229 - protein, 113 aa, Homo sapiens; identical to SEQ ID NO: 221

SEQ ID NO: 230 - protein, 12 aa, Homo sapiens
QSVLYSSNNKNY

SEQ ID NO: 231 - skipped

SEQ ID NO: 232 - protein, 9 aa, Homo sapiens
QQFHRIPPT

SEQ ID NO: 233 - protein, 121 aa, Homo sapiens
QVQLLESGPGLVKPSGTLSLTCDVSGDSISSSNWWSWVRQSPGKGLEWIGEIYQSGTTNNNPSLKSRVTISIDKSKNQFSLNLTSVTAADTAVYYCARAPLVFRSYGMDVWGQGTTVTVSS

SEQ ID NO: 234 - protein, 9 aa, Homo sapiens
GDSISSSNW

SEQ ID NO: 235 - protein, 7 aa, Homo sapiens
IYQSGTT

SEQ ID NO: 236 - protein, 14 aa, Homo sapiens
ARAPLVFRSYGMDV

SEQ ID NO: 237 - protein, 108 aa, Homo sapiens
DIQMTQSPSSLSASVGDRVTITCRASQTIRTYLNWYQQKPGKAPKLLIYDASNLQSGVPSRFSGSGSGTDFTLTISSLEPEDFATYYCQQSYITPPWTFGQGTRVEIK

SEQ ID NO: 238 - protein, 6 aa, Homo sapiens
QTIRTY

SEQ ID NO: 239 - skipped

SEQ ID NO: 240 - protein, 10 aa, Homo sapiens
QQSYITPPWT

SEQ ID NO: 241 - protein, 123 aa, Homo sapiens
QVQLVQSGTEVKKPGSSVKVSCRASGGTLSSYSVSWVRQAPGQGLEWMGRIIPILDKKTYAQKYQGRVTIIADTSTSTVYMELSSLKSEDSAVYYCTTFDYDTSGQRDQHDYWGQGTLVTVSS

SEQ ID NO: 242 - protein, 8 aa, Homo sapiens
GGTLSSYS

SEQ ID NO: 243 - protein, 8 aa, Homo sapiens
IIPILDKK

SEQ ID NO: 244 - protein, 16 aa, Homo sapiens
TTFDYDTSGQRDQHDY

SEQ ID NO: 245 - protein, 113 aa, Homo sapiens
DIVLTQSPDSLAVSLGERATINCKSSQSVLYRSSNQNYLTWYQQKPGQPPKLLIYWASTRESGVPDRFSGSGSGTDFTLTISSLQAEDVAVYFCQQFYTTPFTFGPGTRVDIK

SEQ ID NO: 246 - protein, 12 aa, Homo sapiens
QSVLYRSSNQNY

SEQ ID NO: 247 - skipped

SEQ ID NO: 248 - protein, 9 aa, Homo sapiens
QQFYTTPFT

SEQ ID NO: 249 - protein, 121 aa, Homo sapiens
QVQLQESGPGLVKPSGTLSLTCAVSGDSISSSNWWSWVRQPPGKGLEWIGEIYQSGTTNNNPSLKSRVTISIDKSKNQFSLNLTSVTAADTAVYYCARAPLVFRSYGMDVWGQGTTVTVSS

SEQ ID NO: 250 - protein, 9 aa, Homo sapiens
GDSISSSNW

SEQ ID NO: 251 - protein, 7 aa, Homo sapiens
IYQSGTT

SEQ ID NO: 252 - protein, 14 aa, Homo sapiens
ARAPLVFRSYGMDV

SEQ ID NO: 253 - protein, 108 aa, Homo sapiens
DIQMTQSPSSLSASVGDRVTITCRASQTIRTYLNWYQQKPGKAPKLLIYDASNLQSGVPSRFSGSGSGTDFTLTISSLEPEDFATYYCQQSYITPPWTFGQGTKVEIK

SEQ ID NO: 254 - protein, 6 aa, Homo sapiens
QTIRTY

SEQ ID NO: 255 - skipped

SEQ ID NO: 256 - protein, 10 aa, Homo sapiens
QQSYITPPWT

SEQ ID NO: 257 - protein, 123 aa, Homo sapiens
QVQLVQSGTEVKKPGSSVKVSCRASGGTLSSYSVSWVRQAPGQGLEWMGRIIPILDKKTYAQKYQGRVTIIADTSTSTVYMELSSLKSDDSAVYYCTTFDYDTSGQRDQHDYWGQGTPVTVSS

SEQ ID NO: 258 - protein, 8 aa, Homo sapiens
GGTLSSYS

SEQ ID NO: 259 - protein, 8 aa, Homo sapiens
IIPILDKK

SEQ ID NO: 260 - protein, 16 aa, Homo sapiens
TTFDYDTSGQRDQHDY

SEQ ID NO: 261 - protein, 113 aa, Homo sapiens
DIVLTQSPDSLAVSLGERATINCKSSQSVLYSSSNQNYLTWYQQKPGQPPKLLIFWASTRESGVPDRFSGSGSGTDFTLTISSLQPEDVAVYFCHQFYSTPFTFGPGTRVDIK

SEQ ID NO: 262 - protein, 12 aa, Homo sapiens
QSVLYSSSNQNY

SEQ ID NO: 263 - skipped

SEQ ID NO: 264 - protein, 9 aa, Homo sapiens
HQFYSTPFT

SEQ ID NO: 265 - protein, 126 aa, Homo sapiens
QVQLVESGGGVVQPGRSLRLSCEASGFNFNNYGMHWVRQAPGKGLEWVAVISYEGSKKYYEDSVRGRFTISRDFSKNTLYLQMNSLRAEDTAVYYCAKANEIFWLGQFKNDALDFWGQGTMVTVSS

SEQ ID NO: 266 - protein, 8 aa, Homo sapiens
GFNFNNYG

SEQ ID NO: 267 - protein, 8 aa, Homo sapiens
ISYEGSKK

SEQ ID NO: 268 - protein, 19 aa, Homo sapiens
AKANEIFWLGQFKNDALDF

SEQ ID NO: 269 - protein, 110 aa, Homo sapiens
QSVLTQPPSASGTPGQRVAVSCSGSSSNIGTNFVNWYQHLPRTAPKLLIYRDNQRPSGVPDRFSGSKSGTSASLAISGLQSEDEADYYCAVWDDSLNGQVFGGGTKVTVL

SEQ ID NO: 270 - protein, 8 aa, Homo sapiens
SSNIGTNF

SEQ ID NO: 271 - skipped

SEQ ID NO: 272 - protein, 11 aa, Homo sapiens
AVWDDSLNGQV

SEQ ID NO: 273 - protein, 123 aa, Homo sapiens
QVQLVQSGTEVKKPGSSVKVSCRASGGTLSSYSVSWVRQAPGQGLEWMGRIIPILDKKTYAQKYQGRVTILADTSTSTVYMELSSLKSDDSAVYFCTTFDYDTSGQRDQHDYWGQGTPVTVSS

SEQ ID NO: 274 - protein, 8 aa, Homo sapiens
GGTLSSYS

SEQ ID NO: 275 - protein, 8 aa, Homo sapiens
IIPILDKK

SEQ ID NO: 276 - protein, 16 aa, Homo sapiens
TTFDYDTSGQRDQHDY

SEQ ID NO: 277 - protein, 113 aa, Homo sapiens; identical to SEQ ID NO: 261

SEQ ID NO: 278 - protein, 12 aa, Homo sapiens
QSVLYSSSNQNY

SEQ ID NO: 279 - skipped

SEQ ID NO: 280 - protein, 9 aa, Homo sapiens
HQFYSTPFT

SEQ ID NO: 281 - protein, 124 aa, Homo sapiens
QVQLVESGGGVVQPGRSLRLSCAASGFTFSDHGMHWVRQAPGKGLQWVALIWYDGSNMFYRDSVKGRFTISRDNSKNTVYLEMSSLRAEDTALYYCARGTDVWSGLSLDALDVWGQGTMVTVSP

SEQ ID NO: 282 - protein, 8 aa, Homo sapiens
GFTFSDHG

SEQ ID NO: 283 - protein, 8 aa, Homo sapiens
IWYDGSNM

SEQ ID NO: 284 - protein, 17 aa, Homo sapiens
ARGTDVWSGLSLDALDV

SEQ ID NO: 285 - protein, 113 aa, Homo sapiens
DIVMTQSPLSLPVTPGEPASISCRSSQSLLHYNGYNYLDWYLQKPGQSPRLLIYLGSNRASGVPDRFSGSGSGTDFTLKISRVEAEDVGVYYCMQALQLNLITFGQGTRLEIK

SEQ ID NO: 286 - protein, 11 aa, Homo sapiens
QSLLHYNGYNY

SEQ ID NO: 287 - skipped

SEQ ID NO: 288 - protein, 10 aa, Homo sapiens
MQALQLNLIT

SEQ ID NO: 289 - protein, 117 aa, Homo sapiens
QVRLQESGPGLVKPSETLSVTCTVSGVSISNYYWSWIRQPPGKGLEWIGYISDTGSTNYSPSLQSRVTISVDMSKNQVSLEVTSVTATDTAVYYCARTTTSVPFDYWGKGVLVTVSS

SEQ ID NO: 290 - protein, 8 aa, Homo sapiens
GVSISNYY

SEQ ID NO: 291 - protein, 7 aa, Homo sapiens
ISDTGST

SEQ ID NO: 292 - protein, 11 aa, Homo sapiens
ARTTTSVPFDY

SEQ ID NO: 293 - protein, 107 aa, Homo sapiens
SYELTQPPSVSVSPGQVARITCSGEALPKKYAYWYQHKPGQFPLLVIHKDSERPSGIPERFSGSSSGTVVTLTISGVQAEDEADYFCLSTDGSTTWVFGGGTKLTVL

SEQ ID NO: 294 - protein, 6 aa, Homo sapiens
ALPKKY

SEQ ID NO: 295 - skipped

SEQ ID NO: 296 - protein, 10 aa, Homo sapiens
LSTDGSTTWV

SEQ ID NO: 297 - protein, 128 aa, Homo sapiens
QMQLHESGPGLVKPSQTLSLTCAVSGGSISSDGYYWNWVRQHPGKGLELIAYIYYNGRPYYNPSLRSRVNISLDTSMNQFSLRMKSLTAADTAVYYCARGRLGYCAGGSCFLESFDYWGQGALVTVSS

SEQ ID NO: 298 - protein, 10 aa, Homo sapiens
GGSISSDGYY

SEQ ID NO: 299 - protein, 7 aa, Homo sapiens
IYYNGRP

SEQ ID NO: 300 - protein, 20 aa, Homo sapiens
ARGRLGYCAGGSCFLESFDY

SEQ ID NO: 301 - protein, 107 aa, Homo sapiens
EIVMTQSPATLSVSPGETATLSCRASESISSNLAWYQQKPGQAPRLLIYGASTRATGIPARFSGSGSGTEFALTISSLQAEDFAVYSCQQYHNWLLTFGGGTKVEIK

SEQ ID NO: 302 - protein, 6 aa, Homo sapiens
ESISSN

SEQ ID NO: 303 - skipped

SEQ ID NO: 304 - protein, 9 aa, Homo sapiens
QQYHNWLLT

SEQ ID NO: 305 - protein, 118 aa, Homo sapiens
EVQLVESGGGLVQPGRSLTLSCSGSGFTFGDYSMSWVRQAPGKGLEWLGFIRSQTYGGTTQYAASVQGRFTILRDDSKSIAYLQMNSLKTEDTAVYYCTRNYGVGDYWGQGTLVTVSS

SEQ ID NO: 306 - protein, 8 aa, Homo sapiens
GFTFGDYS

SEQ ID NO: 307 - protein, 10 aa, Homo sapiens
IRSQTYGGTT

SEQ ID NO: 308 - protein, 9 aa, Homo sapiens
TRNYGVGDY

SEQ ID NO: 309 - protein, 114 aa, Homo sapiens
DIVMTQSPASLAVSLGERATIHCRSSLSVLNSSNNKNYLAWYQQKAGQPPKLLMYWASTRESGVPERFSGSGSGTDFTLTISSLEAGDVAVYYCQQYYDKLSLTFGGGTKVEIK

SEQ ID NO: 310 - protein, 12 aa, Homo sapiens
LSVLNSSNNKNY

SEQ ID NO: 311 - skipped

SEQ ID NO: 312 - protein, 10 aa, Homo sapiens
QQYYDKLSLT

SEQ ID NO: 313 - protein, 128 aa, Homo sapiens
QVQLQESGPGLVKASQTLSLTCAVSGGSISSGDYYWTWIRSHPGKGLEWIAYIYYNGRTYYNPSLKSRLTISIDRSKNEFSLKVRSVTAADSAAYYCARGRLGFCAAGSCFLDSLDFWGQGTLVTVSS

SEQ ID NO: 314 - protein, 10 aa, Homo sapiens
GGSISSGDYY

SEQ ID NO: 315 - protein, 7 aa, synthetic construct
IYYNGRT

SEQ ID NO: 316 - protein, 20 aa, Homo sapiens
ARGRLGFCAAGSCFLDSLDF

SEQ ID NO: 317 - protein, 107 aa, Homo sapiens
EIVMTQSPATLSVSPGERVTLSCRASESISNNLAWYQQKPGQAPRLLIYGASTRATGIPARFSGSGSGTEFALIISSLQSEDFAVYFCQQYYNWLLTFGGGTKVEIK

SEQ ID NO: 318 - protein, 6 aa, Homo sapiens
ESISNN

SEQ ID NO: 319 - skipped

SEQ ID NO: 320 - protein, 9 aa, Homo sapiens
QQYYNWLLT

SEQ ID NO: 321 - protein, 128 aa, Homo sapiens
QMQLQESGPGLVKPSQTLSLTCAVSGGSISSDGYYWNWVRQHPGKGLELIAYIYYNGRPYYNPSLRSRVNISLDTSMNQFSLRVKSLTAADTAVYYCARGRLGYCAAGSCFLDSFDYWGQGALVTVSS

SEQ ID NO: 322 - protein, 10 aa, Homo sapiens
GGSISSDGYY

SEQ ID NO: 323 - protein, 7 aa, Homo sapiens
IYYNGRP

SEQ ID NO: 324 - protein, 20 aa, Homo sapiens
ARGRLGYCAAGSCFLDSFDY

SEQ ID NO: 325 - protein, 107 aa, Homo sapiens
EIVMTQYPATLSVSPGETATLSCRASESISRNLAWYQQKPGQAPRLLIYGASTRATGIPARFSGSGSGTEFALTISSLQAEDFAVYSCQQYHNWLLTFGGGTKVEIK

SEQ ID NO: 326 - protein, 6 aa, Homo sapiens
ESISRN

SEQ ID NO: 327 - skipped

SEQ ID NO: 328 - protein, 9 aa, Homo sapiens
QQYHNWLLT

SEQ ID NO: 329 - protein, 117 aa, Homo sapiens
RVQLQESGPGLVKPSETLSLTCTVSGGSITDDYWNWIRQPPGKGLEWIGYVHDSGTTNYNPSLKSRVTLSLHTSGNKFSLRLTSVTAADTAMYYCARTTTTFPFDSWGQGTLVIVSS

SEQ ID NO: 330 - protein, 8 aa, Homo sapiens
GGSITDDY

SEQ ID NO: 331 - protein, 7 aa, Homo sapiens
VHDSGTT

SEQ ID NO: 332 - protein, 11 aa, Homo sapiens
ARTTTTFPFDS

SEQ ID NO: 333 - protein, 107 aa, Homo sapiens
SYELTQPPSVSVSLGQMARITCSGEALPKKYVYWYQQKPGQFPVLVIYKDRERPSGIPERFSGSSSGTIVTLTISGVQAEDEADYYCLSVDSTANWVFGGGTKLTVL

SEQ ID NO: 334 - protein, 6 aa, Homo sapiens
ALPKKY

SEQ ID NO: 335 - skipped

SEQ ID NO: 336 - protein, 10 aa, Homo sapiens
LSVDSTANWV

SEQ ID NO: 337 - protein, 128 aa, Homo sapiens
QVYLQESGPGLVKPSQTLSLTCAVSGGSIRSDGYYWTWIRQHPGKGLEWIGNTYYNGNTYYNPSLRSRVTTSLDTSKNQFSLKVVSMTGADTAVYYCARGRLGFCAGGACFLDTLDYWGQGTLVTVSS

SEQ ID NO: 338 - protein, 10 aa, Homo sapiens
GGSIRSDGYY

SEQ ID NO: 339 - protein, 7 aa, Homo sapiens
TYYNGNT

SEQ ID NO: 340 - protein, 20 aa, Homo sapiens
ARGRLGFCAGGACFLDTLDY

SEQ ID NO: 341 - protein, 107 aa, Homo sapiens
EIVMTQSPVTLSVSPGERVTLSCRADQSVNNNLAWYQQKPGQAPRLLIHGASTRATGIPARFSGSGSGTEFTLTISSLQSEDFAIYYCQQYYNWLLTFGGGTKLEIK

SEQ ID NO: 342 - protein, 6 aa, Homo sapiens
QSVNNN

SEQ ID NO: 343 - skipped

SEQ ID NO: 344 - protein, 9 aa, Homo sapiens
QQYYNWLLT

SEQ ID NO: 345 - protein, 124 aa, Homo sapiens
QVQLVESGGGVVQPGRSLRLSCSASGFTFSDYGMNWVRQAPGKGLEWVAMIWYDGSNYFYRDSVKGRFAISRDNSKNMVFLQMDNLRAEDTAVYFCARGTHFWSGLSLDAFDMWGQGTVVTVSS

SEQ ID NO: 346 - protein, 8 aa, Homo sapiens
GFTFSDYG

SEQ ID NO: 347 - protein, 8 aa, Homo sapiens
IWYDGSNY

SEQ ID NO: 348 - protein, 17 aa, Homo sapiens
ARGTHFWSGLSLDAFDM

SEQ ID NO: 349 - protein, 113 aa, Homo sapiens
EIVMTQSPLSLPVTPGEPASISCRSSRSLLHYNGHNYLDWYLQKPGQSPQLLIYLGSNRAPGVPDRFSGSGSGTDFTLKISRVEAEDVGVYYCMQALQLNLITFGQGTRLEIK

SEQ ID NO: 350 - protein, 11 aa, Homo sapiens
RSLLHYNGHNY

SEQ ID NO: 351 - skipped

SEQ ID NO: 352 - protein, 10 aa, Homo sapiens
MQALQLNLIT

SEQ ID NO: 353 - protein, 128 aa, Homo sapiens
QVQLQESGPGLVKPSQPLSLTCAVSGDSISSGGNYWSWIRQHPGKGLECIAYFYYTGRTYYNPSLKSRVTISLDTSKNQFSLKVSSVTAADTAVYFCARGRLGHCGGGSCFLDPLDYWGQGSLVTVSS

SEQ ID NO: 354 - protein, 10 aa, Homo sapiens
GDSISSGGNY

SEQ ID NO: 355 - protein, 7 aa, Homo sapiens
FYYTGRT

SEQ ID NO: 356 - protein, 20 aa, Homo sapiens
ARGRLGHCGGGSCFLDPLDY

SEQ ID NO: 357 - protein, 107 aa, Homo sapiens
EIVMTQSPATLSVSPGERATLSCRASERISSNLAWSQHKVGQAPRLLIYGASTRATGIPARFSGSGSGTEFTLTISSLQSEDFAVYYCHQYHNWLLTFGGGTKVEIK

SEQ ID NO: 358 - protein, 6 aa, Homo sapiens
ERISSN

SEQ ID NO: 359 - skipped

SEQ ID NO: 360 - protein, 9 aa, Homo sapiens
HQYHNWLLT

SEQ ID NO: 361 - protein, 117 aa, Homo sapiens
QVQLQESGPGLVKPSETLSLTCTVSGGSISSFYWSWIRQPPGKRLEWIGYIYYRGSTNYNPSLKSRVTMSVDTSKNQFSLKLSSVTAADTAVYYCARELPNNWLDPWGQGTLVTVSS

SEQ ID NO: 362 - protein, 8 aa, Homo sapiens
GGSISSFY

SEQ ID NO: 363 - protein, 7 aa, Homo sapiens
IYYRGST

SEQ ID NO: 364 - protein, 11 aa, Homo sapiens
ARELPNNWLDP

SEQ ID NO: 365 - protein, 113 aa, Homo sapiens
DIVMTQSPDSLAVSLGERATINCKSSQSILYNSNNKNYLAWYQQKPGQPPKLLIYWASTRESGVPDRFSGSGSGTDFTLTISSLQAEDVAVYYCQQYHLTPWTFGQGTKVEVT

SEQ ID NO: 366 - protein, 12 aa, Homo sapiens
QSILYNSNNKNY

SEQ ID NO: 367 - skipped

SEQ ID NO: 368 - protein, 9 aa, Homo sapiens
QQYHLTPWT

SEQ ID NO: 369 - protein, 119 aa, Homo sapiens
QLQLQESGSGLVKPSQTLSLTCVVSGDSISSGGYSWSWIRQQPGKALEWIGYIYHSGVTNYNPSLKSRVTISVDRSTNQFSLKLSSVTAADTAVYYCARDGKYNWFDPWGQGILVTVSS

SEQ ID NO: 370 - protein, 10 aa, Homo sapiens
GDSISSGGYS

SEQ ID NO: 371 - protein, 7 aa, Homo sapiens
IYHSGVT

SEQ ID NO: 372 - protein, 11 aa, Homo sapiens
ARDGKYNWFDP

SEQ ID NO: 373 - protein, 113 aa, Homo sapiens
DIVMTQSPDSLAVSLGERAVINCKSSQNVLYTSNNKNYLAWYQQKAGRPPKLLIYWASTRESGVPDRFSGSGSGTDFTLTISSLQAEDVAVYYCQQYFDTPWTFGQGTKVEIK

SEQ ID NO: 374 - protein, 12 aa, Homo sapiens
QNVLYTSNNKNY

SEQ ID NO: 375 - skipped

SEQ ID NO: 376 - protein, 9 aa, Homo sapiens
QQYFDTPWT

SEQ ID NO: 377 - protein, 125 aa, Homo sapiens
QVQLRESGPGVVKPSGTLSLTCTVSGDFINNNRWWTWVRQSPGKGLEWIGEIYHSGSTNYNPSLKGRVTISVDKSKNHFSLKLTSVTAADTAMYYCARGGYCTVGDCHDNWFDPWGQGTLVTVSS

SEQ ID NO: 378 - protein, 9 aa, Homo sapiens
GDFINNNRW

SEQ ID NO: 379 - protein, 7 aa, Homo sapiens
IYHSGST

SEQ ID NO: 380 - protein, 18 aa, Homo sapiens
ARGGYCTVGDCHDNWFDP

SEQ ID NO: 381 - protein, 107 aa, Homo sapiens
DIQMTQSPSTLSASVGDRVTITCRASQNINSWLAWYQQKPGKAPKVLIYKASSLESGVPSRFSGSVSGTEFTLTISSLQPDDFATYYCQQYSTLSWTFGQGTKVDIK

SEQ ID NO: 382 - protein, 6 aa, Homo sapiens
QNINSW

SEQ ID NO: 383 - skipped

SEQ ID NO: 384 - protein, 9 aa, Homo sapiens
QQYSTLSWT

SEQ ID NO: 385 - protein, 120 aa, Homo sapiens
EVQLVQSGAEVKKPGESLRISCRGSGYSFTNYWISWVRQMPGKGLEWMGRIDPTDSYTNYSPSFQGHVTISTDKSISTAYLQWSSLKASDTAMYYCARHKNGMIHEYDYWGQGILVTVSS

SEQ ID NO: 386 - protein, 8 aa, Homo sapiens
GYSFTNYW

SEQ ID NO: 387 - protein, 8 aa, Homo sapiens
IDPTDSYT

SEQ ID NO: 388 - protein, 13 aa, Homo sapiens
ARHKNGMIHEYDY

SEQ ID NO: 389 - protein, 103 aa, Homo sapiens
DIQLTQSPSTLSASVGDRVTITCRASQSIRSWLAWYQQKPGKAPKLLIYRASSLESGVPSRFSGSGSGTEFTLTISSLQPDDFATYYCQQYGTFGPGTKVDIR

SEQ ID NO: 390 - protein, 6 aa, Homo sapiens
QSIRSW

SEQ ID NO: 391 - skipped

SEQ ID NO: 392 - protein, 5 aa, Homo sapiens
QQYGT

SEQ ID NO: 393 - protein, 124 aa, Homo sapiens
RVHLVESGGGVVQPGRSLRLSCATSGFTFSNYAIHWVRQAPGKGLEWVAAVSDDGNSIFYAQSVKGRFTTSRDNSKSTLYLQMNGLRSEDTAVYYCARAQYSDSWGLGNWIDTWGQGTLVTVSS

SEQ ID NO: 394 - protein, 8 aa, Homo sapiens
GFTFSNYA

SEQ ID NO: 395 - protein, 8 aa, Homo sapiens
VSDDGNSI

SEQ ID NO: 396 - protein, 17 aa, Homo sapiens
ARAQYSDSWGLGNWIDT

SEQ ID NO: 397 - protein, 113 aa, Homo sapiens
DIVMTQSPDSLAVSLGERTTINCKSSQSVLYSSNNKNYLAWYQQKPGQAPKVLLYWASTRESGVPDRFSGSGSGTDFNLTISSLQAEDVAVYYCHQYYSPPQTFGQGTKVEIK

SEQ ID NO: 398 - protein, 12 aa, Homo sapiens
QSVLYSSNNKNY

SEQ ID NO: 399 - skipped

SEQ ID NO: 400 - protein, 9 aa, Homo sapiens
HQYYSPPQT

SEQ ID NO: 401 - protein, 124 aa, Homo sapiens
QVQVVESGGGVVQPGGSLRLSCATSGFTFSSYAIHWVRQAPGKGLEWVAAVSDDGNSIFYAQSVKGRFSTSRDNSKNTLYLQMNGLRTEDTAIYYCARADYSDSWGLGNWIDTWGQGTLVTVSS

SEQ ID NO: 402 - protein, 8 aa, Homo sapiens
GFTFSSYA

SEQ ID NO: 403 - protein, 8 aa, Homo sapiens
VSDDGNSI

SEQ ID NO: 404 - protein, 17 aa, Homo sapiens
ARADYSDSWGLGNWIDT

SEQ ID NO: 405 - protein, 113 aa, Homo sapiens
DIVMTQSPDSLAVSLGERATIYCKSSQSVLYSSNNKNYLAWYQQKPGQPPKVLIYWASTRESGVPDRFTGSGSGTDFSLTISSLHPEDVAVYYCHQYYSPPQTFGQGTKVEIK

SEQ ID NO: 406 - protein, 12 aa, Homo sapiens
QSVLYSSNNKNY

SEQ ID NO: 407 - skipped

SEQ ID NO: 408 - protein, 9 aa, Homo sapiens
HQYYSPPQT

SEQ ID NO: 409 - protein, 120 aa, Homo sapiens
EVQLVQSGAEVKKPGESLRISCKGSGYSFSNSWITWVRQMPGKGLEWVGRIDTRDSRTAYSPSFEGHVFFSADRSSDTVYVQWRSLRASDTAVYYCTTGAGVVIAASYFWGQGTLVTVSS

SEQ ID NO: 410 - protein, 8 aa, Homo sapiens
GYSFSNSW

SEQ ID NO: 411 - protein, 8 aa, Homo sapiens
IDTRDSRT

SEQ ID NO: 412 - protein, 13 aa, Homo sapiens
TTGAGVVIAASYF

SEQ ID NO: 413 - protein, 110 aa, Homo sapiens
QSALTQPPSASGSPGQPVTISCTGTSSDVGGYEYVSWYQQLPGKVPTLIIHEVSKRPSGVPDRFSGSKSGNTASLTVSGLQAEDEADYYCSSYSADSSIIFGGGTKLTVL

SEQ ID NO: 414 - protein, 9 aa, Homo sapiens
SSDVGGYEY

SEQ ID NO: 415 - skipped

SEQ ID NO: 416 - protein, 10 aa, Homo sapiens
SSYSADSSII

SEQ ID NO: 417 - protein, 125 aa, Homo sapiens
QVQLQESGPGVVKPSGTLSLTCAVSGDFSNNNRWWTWVRQSPGKGLEWIGEIHQSGSTNYNPSLKSRITISVDKSKNQFSLKLTSVTAADTAVYYCARGGYCSVGDCHDNWFDPWGQGTLVTVSS

SEQ ID NO: 418 - protein, 9 aa, Homo sapiens
GDFSNNNRW

SEQ ID NO: 419 - protein, 7 aa, Homo sapiens
IHQSGST

SEQ ID NO: 420 - protein, 18 aa, Homo sapiens
ARGGYCSVGDCHDNWFDP

SEQ ID NO: 421 - protein, 107 aa, Homo sapiens
DIQMTQSPSTLSASVGDRVTITCRASQTINSWLAWYQQKPGKAPKVLIYKASSLESGVPSRFSGSVSGTEFTLTISSLQPDDFATYYCQQYSSLSWTFGQGTKVDIK

SEQ ID NO: 422 - protein, 6 aa, Homo sapiens
QTINSW

SEQ ID NO: 423 - skipped

SEQ ID NO: 424 - protein, 9 aa, Homo sapiens
QQYSSLSWT

SEQ ID NO: 425 - protein, 117 aa, Homo sapiens
QVQLQESGPGLVKPSETLFLTCAVSGGSINSSYWSWIRQPPGKGLEWIGYIYYRGSTNYNPSLKSRVTISVDTSKNQFSLKLNSVTAADTAIYYCARENLWNWFDPWGQGILVTVSS

SEQ ID NO: 426 - protein, 8 aa, Homo sapiens
GGSINSSY

SEQ ID NO: 427 - protein, 7 aa, Homo sapiens
IYYRGST

SEQ ID NO: 428 - protein, 11 aa, Homo sapiens
ARENLWNWFDP

SEQ ID NO: 429 - protein, 113 aa, Homo sapiens
DIVMTQSPDSLAVSLGERATINCKSSQSVLYSSNNKNYLAWYQQKPGQPPKLLIYWASIRESGVPDRFSGSGSGTDFTLTISGLQAEDVAVYYCQQYHNTPWTFGQGTKVEIK

SEQ ID NO: 430 - protein, 12 aa, Homo sapiens
QSVLYSSNNKNY

SEQ ID NO: 431 - skipped

SEQ ID NO: 432 - protein, 9 aa, Homo sapiens
QQYHNTPWT

SEQ ID NO: 433 - protein, 124 aa, Homo sapiens
EVQLVQSGAEVKKPGESLKISCKGSGYSFTTFWIGWVRQMPGKGLEWVGVIYPDDSDTRYSPSFQGQVTMSADKSINTAYLQWSSLKASDTAVYYCARRNCDAALCYLGTYDYWGQGTVVTVSS

SEQ ID NO: 434 - protein, 8 aa, Homo sapiens
GYSFTTFW

SEQ ID NO: 435 - protein, 8 aa, Homo sapiens
IYPDDSDT

SEQ ID NO: 436 - protein, 17 aa, Homo sapiens
ARRNCDAALCYLGTYDY

SEQ ID NO: 437 - protein, 111 aa, Homo sapiens
QSALTQPPSASGSPGQSVTISCTGTISDVGRYDFVSWYQHHPGKAPKLIIYEVTKRPSGVPDRFSGSKSGNTASLTVSGLQAEDEADYYCNSYAGHNNFVIFGGGTKLTVL

SEQ ID NO: 438 - protein, 9 aa, Homo sapiens
ISDVGRYDF

SEQ ID NO: 439 - skipped

SEQ ID NO: 440 - protein, 11 aa, Homo sapiens
NSYAGHNNFVI

SEQ ID NO: 441 - protein, 117 aa, Homo sapiens
QVQLQESGPGLVKPSETLSLTCTVSGGSIRSNYWSWIRQPPGKRLEWLGYIYYSGSTNYNPSLKSRVTISVDTSKNQFSLNLSSVTAADTAVYYCVREMPYNWIDPWGQGTLVTVSS

SEQ ID NO: 442 - protein, 8 aa, Homo sapiens
GGSIRSNY

SEQ ID NO: 443 - protein, 7 aa, Homo sapiens
IYYSGST

SEQ ID NO: 444 - protein, 11 aa, Homo sapiens
VREMPYNWIDP

SEQ ID NO: 445 - protein, 113 aa, Homo sapiens
DIVMTQSPDSLAVSLGERATINCKSSQSISYSSNNKNYLAWYQQKPGQPPKLLIYWASTRESGVPDRFSGSGSGTDFTLTISSLQAEDVAVYFCQQYFDTPWTFGQGTKVEIK

SEQ ID NO: 446 - protein, 12 aa, Homo sapiens
QSISYSSNNKNY

SEQ ID NO: 447 - skipped

SEQ ID NO: 448 - protein, 9 aa, Homo sapiens
QQYFDTPWT

SEQ ID NO: 449 - protein, 122 aa, Homo sapiens
EVQLVESGGGLVQPGRSLRLSCAASGFTFDDYAMHWVRQAPGKGLEWVSSISWNSGSIVYADSVTGRCTITRDNAKNSLYLQMNSLRPEDTALYYCAKGYSSSPVVYYMDVWGKGTTVTVSS

SEQ ID NO: 450 - protein, 8 aa, Homo sapiens
GFTFDDYA

SEQ ID NO: 451 - protein, 8 aa, Homo sapiens
ISWNSGSI

SEQ ID NO: 452 - protein, 15 aa, Homo sapiens
AKGYSSSPVVYYMDV

SEQ ID NO: 453 - protein, 103 aa, Homo sapiens
DIQMTQSPSSVSASVGDRVTITCRASQGIAGWFAWYQQKPGKAPKLLIYATSSLQRGVPSRFSGSGSATHFTLTITSLQPEDFATYYCQQANIFGPGTKVDIK

SEQ ID NO: 454 - protein, 6 aa, Homo sapiens
QGIAGW

SEQ ID NO: 455 - skipped

SEQ ID NO: 456 - protein, 5 aa, Homo sapiens
QQANI

SEQ ID NO: 457 - protein, 121 aa, Homo sapiens
QVQLQESGPGQVKPSGTLSLTCAVSGGSINSSNWWSWVRQPPGKGLEWIGEIFHSGNTNYKPSLESRVTISFDKSKKELSLRLTSVTAADTAVYYCATLFCSGGTCYLGDWGQGTLVTVSS

SEQ ID NO: 458 - protein, 9 aa, Homo sapiens
GGSINSSNW

SEQ ID NO: 459 - protein, 7 aa, Homo sapiens
IFHSGNT

SEQ ID NO: 460 - protein, 14 aa, Homo sapiens
ATLFCSGGTCYLGD

SEQ ID NO: 461 - protein, 107 aa, Homo sapiens
EIVLTQSPGTLSVSPGQRATLSCRASESVSSRYVAWYQQKPGQAPRLIIYGASSRATGIPDRFSGSGSGTEFILSISRLEPEDSAVYYCQQYGSSPTFGGGTKVEIK

SEQ ID NO: 462 - protein, 7 aa, Homo sapiens
ESVSSRY

SEQ ID NO: 463 - skipped

SEQ ID NO: 464 - protein, 8 aa, Homo sapiens
QQYGSSPT

SEQ ID NO: 465 - protein, 124 aa, Homo sapiens
QVQLVESGGGVVQPGRSLRLSCAASGFTFSSYAMHWVRQAPGKGLEWVAVISYDGNNAFYADSVKGRFTISRDNSKNTVYVQMDSLRAEETAVYYCARAGYNSNWDLGDALDIWGQGTMVTVSS

SEQ ID NO: 466 - protein, 8 aa, Homo sapiens
GFTFSSYA

SEQ ID NO: 467 - protein, 8 aa, Homo sapiens
ISYDGNNA

SEQ ID NO: 468 - protein, 17 aa, Homo sapiens
ARAGYNSNWDLGDALDI

SEQ ID NO: 469 - protein, 113 aa, Homo sapiens
DIVMTQSPDSLAVSLGERATINCKSSQSVLYSSNNKNYLAWYQQKPGQPPKLLINWASTRESGVPDRFSGSGSGTDFTLTISSLQAEDVAVYYCQQYYSIPQTFGQGTKVEIK

SEQ ID NO: 470 - protein, 12 aa, Homo sapiens
QSVLYSSNNKNY

SEQ ID NO: 471 - skipped

SEQ ID NO: 472 - protein, 9 aa, Homo sapiens
QQYYSIPQT

SEQ ID NO: 473 - protein, 124 aa, Homo sapiens
QVQLVDSGGGAVQPGTSLRLSCAASGFTFNTYAFHWVRQAPGKGLEWVAVISDDGNNKYYADSVKGRFTISRDDSKNTLSLQMDSLRVEDAAVYFCARGSYNSNWSLGEYFDYWGQGSLVTVSS

SEQ ID NO: 474 - protein, 8 aa, Homo sapiens
GFTFNTYA

SEQ ID NO: 475 - protein, 8 aa, Homo sapiens
ISDDGNNK

SEQ ID NO: 476 - protein, 17 aa, Homo sapiens
ARGSYNSNWSLGEYFDY

SEQ ID NO: 477 - protein, 113 aa, Homo sapiens
DIVMTQSPDSLPVSLGERATINCKSSQSVLHSSNNKNYLAWYQQKPGQSPKLLISWASTRESGVPDRFSGSGSGTHFTLTISSLRAEDVAVYYCLQYFNNQYTFGQGTKLEIK

SEQ ID NO: 478 - protein, 12 aa, Homo sapiens
QSVLHSSNNKNY

SEQ ID NO: 479 - skipped

SEQ ID NO: 480 - protein, 9 aa, Homo sapiens
LQYFNNQYT

SEQ ID NO: 481 - protein, 118 aa, Homo sapiens
QVQLVQSGAEVKKPGASVMLSCRASGYTFINDYMHWVRQAPGEGFEWMGIINPTGDGTKFAQKFQGRVTMTRDSSTTTVHMELRSLTSGDTAVYFCATGGRQSAFDIWGQGTVVIVSS

SEQ ID NO: 482 - protein, 8 aa, Homo sapiens
GYTFINDY

SEQ ID NO: 483 - protein, 8 aa, Homo sapiens
INPTGDGT

SEQ ID NO: 484 - protein, 11 aa, Homo sapiens
ATGGRQSAFDI

SEQ ID NO: 485 - protein, 114 aa, Homo sapiens
DIVMTQSPLSLPVTPGESASISCRSSQSLLQNNGYNYLDWYLQKPGQSPQLLIYLGSYRASGVPDRFSGSGSGTDFTLKISRVEAEDVGVYYCVQAQQAPPLYTFGQGTKLQIK

SEQ ID NO: 486 - protein, 11 aa, Homo sapiens
QSLLQNNGYNY

SEQ ID NO: 487 - skipped

SEQ ID NO: 488 - protein, 11 aa, Homo sapiens
VQAQQAPPLYT

SEQ ID NO: 489 - protein, 21 aa, SARS-CoV-2
LQPELDSFKEELDKYFKNHTS

SEQ ID NO: 490 - protein, 12 aa, SARS-CoV-2
LDSFKEELDKYF

SEQ ID NO: 491 - protein, 15 aa, Synthetic construct
PSKPSKRSFIEDLLF

SEQ ID NO: 492 - protein, 15 aa, synthetic construct
PSKRSFIEDLLFNKV

SEQ ID NO: 493 - protein, 15 aa, synthetic construct
RSFIEDLLFNKVTLA

SEQ ID NO: 494 - protein, 15 aa, synthetic construct
QPELDSFKEELDKYF

SEQ ID NO: 495 - protein, 15 aa, synthetic construct
LDSFKEELDKYFKNH

SEQ ID NO: 496 - protein, 15 aa, synthetic construct
FKEELDKYFKNHTSP

SEQ ID NO: 497 - protein, 14 aa, synthetic construct
SFIEDLLFNKVTLA

SEQ ID NO: 498 - protein, 9 aa, synthetic construct
RSFIEDLLF

SEQ ID NO: 499 - protein, 22 aa, Coronavirus
VPEYIDVNKTLQELSYKLPNYT

SEQ ID NO: 500 - protein, 22 aa, Coronavirus
IPDYVDVNKTLQEFAQNLPKYV

SEQ ID NO: 501 - protein, 22 aa, Mouse Hepatitis Virus
IPNPPDFKEELDKWFKNQTSIA

SEQ ID NO: 502 - protein, 22 aa, Coronavirus
IPNLPDFKEELDQWFKNQTSVA

SEQ ID NO: 503 - protein, 22 aa, Coronavirus
IPNLSDFEAELSLWFKNHTSIA

SEQ ID NO: 504 - protein, 22 aa, Coronavirus
QPELDSFKEELDKYFKNHTSPD

SEQ ID NO: 505 - protein, 24 aa, Coronavirus
LENSTDVDFKDELEEFFKNVTSHG

SEQ ID NO: 506 - protein, 24 aa, Coronavirus
LSNSTDLDFKEELEEFFKNVSSQG

SEQ ID NO: 507 - protein, 24 aa, Middle East Respiratory Syndrome Coronavirus
LGNSTGIDFQDELDEFFKNVSTSI

SEQ ID NO: 508 - protein, 23 aa, Coronavirus
VDTSSFNFTEEFEKWYVNQSHIF

SEQ ID NO: 509 - protein, 22 aa, Coronavirus
PPSSYDFTEEFNKWYKNHSSYF

SEQ ID NO: 510 - protein, 22 aa, Coronavirus
VPGDYDFQKEFDKFYKNLSTIF

SEQ ID NO: 511 - protein, 22 aa, Coronavirus
MPELEDFKQELEEIRKNFSTGL

SEQ ID NO: 512 - protein, 34 aa, Coronavirus
KRKYRSAIEDLLFDKVVTSGLGTVDEDYKRCTGG

SEQ ID NO: 513 - protein, 34 aa, Coronavirus
RVAGRSAIEDILFSKLVTSGLGTVDADYKKCTKG

SEQ ID NO: 514 - protein, 34 aa, Coronavirus
RIAGRSALEDLLFSKVVTSGLGTVDVDYKSCTKG

SEQ ID NO: 515 - protein, 32 aa, Coronavirus
SSSRSLLEDLLFNKVKLSDVGFVEAYNNCTGG

SEQ ID NO: 516 - protein, 33 aa, Coronavirus
KASSRSAIEDLLFDKVKLSDVGFVEAYNNCTGG

SEQ ID NO: 517 - protein, 33 aa, Mouse Hepatitis Virus
AIRGRSAIEDLLFDKVKLSDVGFVEAYNNCTGG

SEQ ID NO: 518 - protein, 33 aa, Coronavirus
KPTKRSFIEDLLFNKVTLADAGFMKQYGDCLGD

SEQ ID NO: 519 - protein, 33 aa, Coronavirus
KPSKRSFIEDLLFNKVTLADAGFIKQYGDCLGD

SEQ ID NO: 520 - protein, 33 aa, Coronavirus
KPTKRSFIEDLLFNKVTLADAGFMKQYGECLGD

SEQ ID NO: 521 - protein, 33 aa, Coronavirus
SSSYRSAIEDLLFDKVTIADPGYMQGYDDCMKQ

SEQ ID NO: 522 - protein, 33 aa, Coronavirus
SRSARSAIEDLLFDKVTIADPGYMQGYDDCMQQ

SEQ ID NO: 523 - protein, 33 aa, Coronavirus
ATTYRSAFSDLLYDKVRITDPGFMQSYQKCIDS

SEQ ID NO: 524 - protein, 33 aa, Avian Infectious Bronchitis Virus
SPRRRSFIEDLLFTSVESVGLPTDDAYKNCTAG

SEQ ID NO: 525 - protein, 33 aa, Avian Infectious Bronchitis Virus
SRRKRSLIEDLLFTSVESVGLPTNDAYKNCTAG

SEQ ID NO: 526 - protein, 34 aa, Coronavirus
QNQGRSTIEDLLFDKVVTLGVSEVDQNYDKCIAS

SEQ ID NO: 527 - protein, 34 aa, Coronavirus
PRDARSTIEDILFDKVTTVGLGTVDADYDKCTKG

SEQ ID NO: 528 - protein, 34 aa, Coronavirus
KAGGRSAIEDLLFNKVVTNGLGTVDQDYKACSKD

SEQ ID NO: 529 - protein, 34 aa, Coronavirus
KIGEKSVIEDLLFNKVVTNGLGTVDQDYKACSKD

SEQ ID NO: 530 - protein, 34 aa, Coronavirus
RLGGRSAIEDLLFNKVVTSGLGTVDQDYKACSRD

SEQ ID NO: 531 - protein, 34 aa, Coronavirus
KQGGRSAIEDLLFDKVVTNGLGTVDQDYKECTKG

SEQ ID NO: 532 - protein, 34 aa, Coronavirus
VKQGRSALEDLLFTKVVTAGLGTVDADYEKCAKG